(19) United States
(12) Patent Application Publication
(10) Pub. No.: US 2008/0312287 A1
(43) Pub. Date: Dec. 18, 2008

(54) Compound and Methods For the Treatment of Cancer and Malaria

(75) Inventors: Andrew Hamilton, Guilford, CT (US); Frederick Buckner, Seattle, WA (US); Matthew Glenn, Queensland (AU); Wesley Van Voorhis, Seattle, WA (US); Said Sebti, Tampa, FL (US)

Correspondence Address: COLEMAN SUDOL SAPONE, P.C., 714 COLORADO AVENUE, BRIDGE PORT, CT, 06605-1601, US

(73) Assignee: University of Washington, Seattle, WA (US)

(21) Appl. No.: 11/886,105

(22) PCT Filed: Mar. 17, 2006
(86) PCT No.: PCT/US06/09861

§ 371 Date: May 9, 2008

Related U.S. Application Data
(60) Provisional application No. 60/663,370, filed on Mar. 17, 2005.

(51) Int. Cl. A61K 31/4439; C07D 403/02; A61K 31/4178; A61P 35/00; A61P 33/06; C07D 401/02

(52) U.S. Cl. 514/341; 548/312.7; 546/275.1; 514/397

(57) ABSTRACT

Formula (I): Where R¹ is an optionally substituted C₃-C₁₂ hydrocarbyl group (preferably a cyclic alkyl group), an optionally substituted heterocyclic group, an optionally substituted aromatic group or an optionally substituted heteroaromatic group; R is a C(O)_{y}R′ group (preferably forming an optionally substituted C₂-C₅ acyl group), or a S(O)ₓR′ group, where y is 0 or 1 and x is 0, 1 or 2 and R′ is H or an optionally substituted C₁-C₁₂ alkyl group, or R′ is an optionally substituted C₅-C₁₂ cycloalkyl group, an optionally substituted heterocyclic group, an optionally substituted aromatic group or an optionally substituted heteroaromatic group; R⁵, R⁶, R⁷, R⁸, R⁹ and R¹⁰ are each independently selected from H, an optionally substituted C₁-C₁₂ hydrocarbyl group, including a C₅-C₁₂ cycloalkyl group, an optionally substituted heterocyclic group, an optionally substituted aromatic group or an optionally substituted heteroaromatic group, or R⁵ and R⁶, R⁷ and R⁸ or R⁹ and R¹⁰ together form a keto (C═O) group; R^{N} is H, an optionally substituted C₁-C₁₂ hydrocarbyl group, an optionally substituted heterocyclic group, an optionally substituted aromatic group, or an optionally substituted heteroaromatic group; A is Formula (II): a Formula (III): group, or a Formula (IV) or Formula (V) group, where Z is N, O or S; R^{a} is H, a C₁-C₁₂ optionally substituted hydrocarbyl group or an optionally substituted aromatic group; n is from 0 to 3; and pharmaceutically acceptable salts thereof. Compounds according to the invention are useful in one or more aspects to inhibit farnesyl transferase, or to treat malaria, neoplasia, a hyperproliferative disease state or arthritis, including rheumaroid arthritis or osteoarthritis.

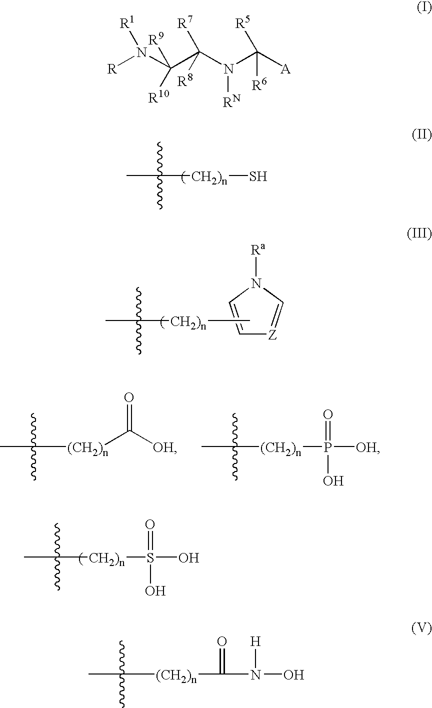

This application claims the benefit of priority from U.S. provisional application no. U.S. 60/633,670, filed Mar. 17, 2005.

The United States government has provided support for this invention in the form of NIH grant GM35208. Consequently, the government retains certain rights in the invention.

### FIELD OF THE INVENTION

The present invention relates to substituted imidazole compounds which exhibit activity against malaria and cancer and methods of treating malaria and cancer in patients.

### BACKGROUND OF THE INVENTION

Malaria is an ancient, infectious disease that continues to inflict suffering and death on a staggering scale. Current estimates indicate that there are 300-500 million acute cases of malaria each year, resulting in one to three million deaths.¹ In the highest risk group, African children under the age of five, malaria claims a young life every 40 seconds. Unfortunately mortality from malaria appears to be increasing, and is almost certainly associated with the increasing resistance of malaria parasites to available drugs.²⁻⁴

Malaria is caused by protazoal parasites of the genus Plasmodium, of which four species are known to cause malaria in humans: falciparum, vivax, malariae, and ovale. The parasites are transmitted through the bite of infected mosquitoes of the genus Anopheles, and following an initial asymptomatic localization and incubation in the liver, the parasites enter circulating erythrocytes and consume hemoglobin and other proteins within the cell. The protozoa replicate inside the blood cells, ultimately inducing cytolysis and release of toxic metabolic byproducts into the blood stream. The clinical symptoms of malaria result exclusively from the erythrocytic stage, and include flu like symptoms, jaundice and anemia. Mortality is almost exclusively attributable to infection by P. falciparum, which produces specific proteins that embed into the cell membrane of the infected erythrocyte. These cells bind to pre-venous capillaries, resulting in obstruction of blood vessels in many areas of the body. Of significant concern is the increasing discovery of P. falciparum resistant to existing drugs (chloroquine, mefloquine, sulfadoxime/pyrimethamine),^{4,5} with strains now reported that are resistant to all known anti-malarial therapies, potentially foreshadowing devastating consequences if new treatments are not identified.

The clear need for new effective anti-malarials is complicated by the resource limitations of the countries most affected, with the overwhelming majority of mortality (˜90%) confined to the world's most impoverished nations.^{1,6} In this setting, the development of new anti-malarial treatments must give critical consideration to the economics of drug development and delivery. In an effort to reduce development costs and accelerate access to new anti-malarials, recent attention has been directed towards identifying anti-malarial activity from agents developed for the treatment of other diseases.⁷⁻¹¹

On recognizing the essential role of prenylation for cellular function in lower eukaryotes,⁷⁻¹² several groups have investigated the anti-malarial potential of inhibitors of farnesyl transfease,^{8,13,14} a recognized key target for the interception of aberrant Ras activity common to many (˜30%) human cancers.¹⁵ Treatment of P. falciparum infected cells with anti-cancer farnesyltransferase inhibitors induces a decrease in farnesylated proteins, and associated lysis of the parasites.¹¹ Animal studies recently demonstrated that closely related derivatives of anti cancer PFT inhibitors cure malaria-infected mice. However, the delivery costs (synthesis and administration) of drugs developed by wealthy nations for the treatment of diseases such as cancer may be prohibitively expensive for third world nations, even in the absence of the associated costs for research and development. In this report we discuss a new series of PFTase inhibitors that have been developed specifically as novel anti-malarial agents, emphasizing simple molecular architecture and straightforward chemical synthesis, as a prerequisite for access to low cost treatment for the third world.

### OBJECTS OF THE INVENTION

It is an object of the invention to provide compounds for use in treating malaria.

It is another object of the invention to provide compounds for use in treating cancer.

It is still a further object of the invention to provide pharmaceutical compositions based upon these compounds.

It is yet an additional object of the invention to provide methods for treating malaria in mammals, primarily humans using compounds according to the present invention.

It is still another object of the invention to provide methods for treating cancer in mammals, including humans using compounds according to the present invention.

It is still yet an additional object of the invention to provide methods for treating hyperproliferative diseases or chronic inflammatory diseases as otherwise disclosed herein.

Any one or more of these and/or other objects of the invention may be gleaned from the description of the invention which follows.

### BRIEF DESCRIPTION OF THE FIGURES

FIG. 1 shows the active site conformation of 8x (colored by atom type), as determined by flexible ligand docking,²² in the homology model of the active site of Plasmodium PFTase (red hydrophobic to blue hydrophilic). Values in parentheses refer to the corresponding residues of rat FTase (pdb: 1JCR).

FIG. 2 shows the chemical steps of synthetic scheme 1. Reagents and conditions: (a) HBTU, DIPEA, DMF; (b) TFA; (c) LAH, THF; (d) Alkylsulfonyl Chloride (R²), TEA, DMF.

2D NOESY of deprotected 5 in d₄-methanol, identifies the close spatial arrangement of protons about the imidazole and aniline, confirming chemoselective alkylation of the aniline nitrogen. Strong cross peaks are observed (highlighted in red) between the methylene protons at c, to the protons at g, and b, while only a weak cross peak is observed between protons at c, to methylene a.

FIG. 3 shows the chemical steps of synthetic scheme 2. Reagents and conditions: (a) 4-Fluorobenzonitrile, TEA, DMSO, 120° C.; (b) 4-X-aniline (X=Br, Ph), NaCNBH₃, Acetic Acid, 3 Å Molecular Sieves, MeOH; (c) LDA, NaH, 5-Chloromethyl-1-methyl-1H-imidazole.HCl, THF, −78° C.; (d) NaH, Alkylbromide (R¹), DMF, 0° C.; (e) TFA; (f) Sulfonyl Chloride (R²), TEA, DMF; (g) Alkylbromide (R¹), CsCO₃, DMF.

FIG. 4 shows the pharmacokinetics of representative compounds according to the present invention. Left: Metabolism of 8d by rat liver microsomes. Right: Average plasma concentrations of inhibitors 8d, 8x, 8g and 8v in three rats or mice after oral garage (Dose: rats 12.5 mg, mice 1 mg).

### BRIEF DESCRIPTION OF THE INVENTION

The present invention relates to compounds according to the structure:

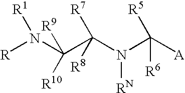

Where R¹ is an optionally substituted C₃-C₁₂ hydrocarbyl group (preferably a cyclic alkyl group), an optionally substituted heterocyclic group, an optionally substituted aromatic group or an optionally substituted heteroaromatic group;
R is a C(O)_{y}R′ group (preferably forming an optionally substituted C₂-C₅ acyl group), or a S(O)ₓR′ group, where y is 0 or 1 and x is 0, 1 or 2 and R′ is H or an optionally substituted C₁-C₁₂ alkyl group, or R′ is an optionally substituted C₅-C₁₂ cycloalkyl group, an optionally substituted heterocyclic group, an optionally substituted aromatic group or an optionally substituted heteroaromatic group;
R⁵, R⁶, R⁷, R⁸, R⁹ and R¹⁰ are each independently selected from H, an optionally substituted C₁-C₁₂ hydrocarbyl group, including a C₅-C₁₂ cycloalkyl group, an optionally substituted heterocyclic group, an optionally substituted aromatic group or an optionally substituted heteroaromatic group, or R⁵ and R⁶, R⁷ and R⁸ or R⁹ and R¹⁰ together form a keto (C═O) group;
R^{N} is H, an optionally substituted C₁-C₁₂ hydrocarbyl group, an optionally substituted heterocyclic group, an optionally substituted aromatic group, or an optionally substituted heteroaromatic group;

### A is or a

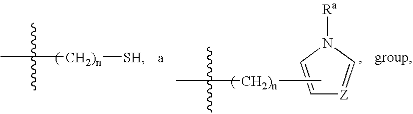

or a

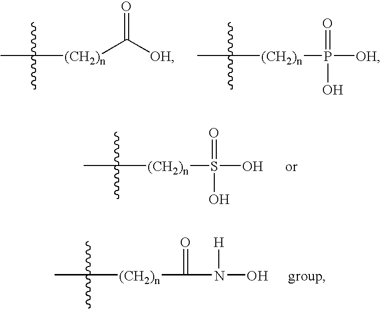

where Z is N, O or S;
R^{a} is H, a C₁-C₁₂ optionally substituted hydrocarbyl group or an optionally substituted aromatic group;
n is from 0 to 3; and pharmaceutically acceptable salts thereof.

In preferred aspects, the present invention relates to compounds according to the structure:

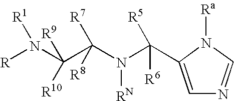

Where R^{a} is H or a C₁-C₆ optionally substituted hydrocarbyl group or an optionally substituted aromatic group;
R¹ is an optionally substituted C₃-C₁₂ hydrocarbyl group, an optionally substituted heterocyclic group, an optionally substituted aromatic group or an optionally substituted heteroaromatic group;
R is a C(O)_{y}R′ group (preferably forming an optionally substituted C₂-C₅ acyl group), or a S(O)ₓR′ group, where y is 0 or 1 and x is 0, 1 or 2 and R′ is H or an optionally substituted C₁-C₁₂ alkyl group, or R′ is an optionally substituted C₅-C₁₂ cycloalkyl group, an optionally substituted heterocyclic group, an optionally substituted aromatic group or an optionally substituted heteroaromatic group;
R⁵, R⁶, R⁷, R⁸, R⁹ and R¹⁰ are each independently selected from H, an optionally substituted C₁-C₃ hydrocarbyl group, or R⁵ and R⁶, R⁷ and R⁸ or R⁹ and R¹⁰ together form a keto (C═O) group; and
R^{N} is H, an optionally substituted C₁-C₁₂ hydrocarbyl group, an optionally substituted heterocyclic group, an optionally substituted aromatic group, or an optionally substituted heteroaromatic group, and pharmaceutically acceptable salts thereof.

In certain further preferred aspects of the invention, R¹ is preferably an optionally substituted alkylene phenyl group (e.g. a benzyl group) or an optionally substituted heterocyclic or optionally substituted heteroaromatic group, R is preferably a C₂-C₅ keto group or an SO₂R′ group where R′ is preferably an optionally substituted phenyl group, or an optionally substituted heteroaromatic group (N-methylimidazole group), R^{a} is an alkyl group, preferably a methyl group, R^{N} is preferably a substituted phenyl group (CN, halogen), and R⁵, R⁶, R⁷, R⁸, R⁹ and R¹⁰ are each independently H or CH₃, or R⁵ and R⁶, R⁷ and R⁸ or R⁹ and R¹⁰ together independently form a keto (C═O) group, and n is 0 or 1.

In another aspect of the invention, pharmaceutical compositions comprise an effective amount of a compound as set forth above, optionally in combination with a pharmaceutically acceptable carrier, additive or excipient.

In a method aspect, the present invention is directed to the inhibition of farnseyl transferase enzyme in a patient or subject, in particular farnseyl transferase in a patient in need of therapy comprising administering to said patient an effective amount of one or more compounds according to the present invention to the patient. The method of inhibiting farnesyl transferase, especially farnesyl transferase in a patient will result in a pharmacological effect consistent with such inhibition in the patient.

The present invention is directed to the treatment of malaria comprising administering to a patient in need of therapy an effective amount of a compound according to the present invention, optionally, in combination with a pharmaceutically acceptable additive, carrier or excipient.

The present invention is also directed to a method for treating tumors and/or cancer in a patient in need of therapy comprising administering to such a patient an effective amount of one or more compounds according to the present invention, optionally in combination with a pharmaceutically acceptable additive, carrier or excipient.

The tumors and/or cancer to be treated with compounds of the present invention include benign and malignant neoplasia, including various cancers such as, stomach, colon, rectal, liver, pancreatic, lung, breast, cervix uteri, corpus uteri, ovary, prostate, testis, bladder, renal, brain/cns, head and neck, throat, Hodgkins disease, non-Hodgkins leukemia, multiple myeloma leukemias, skin melanoma, acute lymphocytic leukemia, acute mylogenous leukemia, Ewings Sarcoma, small cell lung cancer, choriocarcinoma, rhabdomyosarcoma, Wilms Tumor, neuroblastoma, hairy cell leukemia, mouth/pharynx, oesophagus, larynx, melanoma, kidney, lymphoma, among others. Compounds according to the present invention are particularly useful in the treatment of a number of cancers, including those which are drug resistant, including multiple drug resistant cancers.

A method of treating hyperproliferative cell growth and psoriasis and related conditions using one or more of the disclosed compositions are other inventive aspects of the present invention. This method comprises administering to a patient in need of therapy an effective amount of one or more compounds according to the present invention to said patient, optionally in combination with an additive, carrier or excipient.

A method of treating arthritis and chronic inflammatory diseases, including rheumatoid arthritis and osteoarthritis, among others represent other inventive aspects of the present invention. This method comprises administering to a patient in need of therapy an effective amount of one or more compounds according to the present invention to said patient, optionally in combination with an additive, carrier or excipient.

### DETAILED DESCRIPTION OF THE INVENTION

The following terms are used to describe the present invention. Where a term is not specifically defined herein, the term is given the same meaning that one of ordinary skill would ascribe to the term when used within the context of describing the present invention.

The term “patient” or “subject” is used throughout the specification to describe a subject animal, preferably a human, to whom treatment, including prophylactic treatment, with the compounds/compositions according to the present invention is provided. For treatment of those conditions or disease states which are specific for a specific animal such as a human patient, the term patient refers to that specific animal.

The term “compound”, as used herein, unless otherwise indicated, refers to any specific chemical compound disclosed herein and includes in context, tautomers, regioisomers, geometric isomers, and where applicable, optical isomers thereof, as well as pharmaceutically acceptable salts, solvates and polymorphs thereof. Within its use in context, the term compound generally refers to a single compound, but also may include other compounds such as stereoisomers, regioisomers and/or optical isomers (including in some instances, racemic mixtures) as well as specific enantiomers or enantiomerically enriched mixtures of disclosed compounds.

The term “independently” is used herein to indicate that the variable, which is independently applied, varies independently from application to application.

The term “malaria” refers to a disease caused by the presence of the sporozoan Plasmodium (in humans, P. falciparum, P. vivax, P. malariae and P. ovate are causative agents) in humans or other vertebrate red blood cells, usually transmitted to humans by the bite of an infected female mosquito of the genus Anopheles that previously sucked the blood from a person with malaria. Human infection begins with the exoerythrocytic cycle in liver parenchyma cells, followed by a series of erythrocytic schizogenous cycles repeated at regular intervals; production of gametocytes in other red cells provides future gametes for another mosquito infection. The disease is characterized by episodic severe chills and high fever; prostration, occasionally fatal termination. The present invention may be used to treat veterinary (i.e., non-human) forms of malaria as well as human forms of malaria.

The term “neoplasia” is used to describe the pathological process that results in the formation and growth of a neoplasm, i.e., an abnormal tissue that grows by cellular proliferation more rapidly than normal tissue and continues to grow after the stimuli that initiated the new growth cease. Neoplasia exhibits partial or complete lack of structural organization and functional coordination with the normal tissue, and usually form a distinct mass of tissue which may be benign (benign tumor) or malignant (carcinoma). The term “cancer” is used as a general term to describe any of various types of malignant neoplasms, most of which invade surrounding tissues, may metastasize to several sites and are likely to recur after attempted removal and to cause death of the patient unless adequately treated. As used herein, the term cancer is subsumed under the term neoplasia. The term “tumor and/or cancer” is used to describe all types of neoplasia, including benign and malignant. The other conditions and/or disease states which are described herein use standard terms for their description which are well known in the art. Exemplary tumors and/or cancers which may be effectively treated by the present invention include, for example, stomach, colon, rectal, liver, pancreatic, lung, breast, cervix uteri, corpus uteri, ovary, prostate, testis, bladder, renal, brain/cns, head and neck, throat, Hodgkins disease, non-Hodgkins leukemia, multiple myeloma leukemias, skin melanoma, acute lymphocytic leukemia, acute mylogenous leukemia, Ewings Sarcoma, small cell lung cancer, choriocarcinoma, rhabdomyosarcoma, Wilms Tumor, neuroblastoma, hairy cell leukemia, mouth/pharynx, oesophagus, larynx, melanoma, kidney, lymphoma, among others.

The term “tumor” is used to describe a malignant or benign growth or tumefacent.

The term “hyperproliferative disease state” refers to a disease state in which cells are growing in an uncontrolled manner, whether that growth is cancerous or not. Such a disease state may be reflected in psoriasis, genital warts or other hyperproliferative cell growth diseases, including hyperproliferative keratinocyte diseases including hyperkeratosis, ichthyosis, keratoderma or lichen planus, all of which disease states may be treated using compounds according to the present invention.

The term “hydrocarbyl” shall mean within its use in context, a radical containing carbon and hydrogen atoms, preferably containing between 1 and 12 carbon atoms. Such term may also include cyclic groups and unsaturated groups such as aromatic groups, within context. A substituted hydrocarbyl group is a hydrocarbyl group where at least one hydrogen atom is substituted by another moiety, as described below. The term “alkyl” shall mean within its use in context a fully saturated C₁-C₁₂ hydrocarbon linear, branch-chained or cyclic radical, preferably a C₁-C₄, even more preferably a C₁-C₃ linear, branch-chained or cyclic fully saturated hydrocarbon radical. The term “alkenyl” is used to describe a hydrocarbon group, similar to an alkyl group which contains one double bond. Unsaturated hydrocarbyl groups are anticipated for use in the present invention. The terms “alkylene” and “alkenylene” may be used to describe alkyl and alkenyl divalent radicals generally of up to 12 carbon units in length and preferably no greater than about 6 carbon units per length (for example, 1-3 carbon units in length) and may be subsumed under the terms alkyl and alkenyl, especially when referring to substituents or substituted.

The term “aromatic” or “aryl” shall mean within its context a substituted or unsubstituted monovalent carbocyclic aromatic radical having a single ring (e.g., phenyl) or multiple condensed rings (e.g., naphthyl, anthracene, phenanthrene). Other examples include optionally substituted heterocyclic aromatic ring groups (“heteroaromatic” or “heteroaryl”) having one or more nitrogen, oxygen, or sulfur atoms in the ring, such as imidazolyl, furyl, pyrrolyl, pyridyl, thiophene, thiazole, indolyl, quinoline, among numerous others. The preferred aryl group in compounds according to the present invention is a phenyl or a substituted phenyl group.

The term “heterocycle” shall mean an optionally substituted moiety which is cyclic and contains at least one atom other than a carbon atom, such as a nitrogen, sulfur, oxygen or other atom. A heterocycle according to the present invention is an optionally substituted imidazole, a piperazine (including piperazinone), piperidine, furan, pyrrole, imidazole, thiazole, oxazole or isoxazole group. Depending upon its use in context, a heterocyclic ring may be saturated and/or unsaturated.

The term “unsubstituted” shall mean substituted only with hydrogen atoms. The term “substituted” shall mean, within the chemical context of the compound defined, a substituent (each of which substituent may itself be substituted) selected from a hydrocarbyl (which may be substituted itself, preferably with an optionally substituted alkyl or fluoro group, among others), preferably an alkyl (generally, no greater than about 12 carbon units in length), an optionally substituted aryl (which also may be heteroaryl and may include an alkylenearyl or alkyleneheteroaryl), an optionally substituted heterocycle (especially including an alkyleneheterocycle), CF₃, halogen, thiol, hydroxyl, carboxyl, oxygen (to form a keto group), C₁-C₈ alkoxy, CN, nitro, an optionally substituted amine (e.g. an alkyleneamine or a C₁-C₆ monoalkyl or dialkyl amine), C₁-C₈ acyl, C₁-C₈ alkylester, C₁-C₈ alkyleneacyl (keto), C₁-C₈ alkylene ester, carboxylic acid, alkylene carboxylic acid, C₁-C₈ thioester, C₂-C₈ ether, C₁-C₈ thioether, amide (amido or carboxamido), substituted amide (especially mono- or di-alkylamide) or alkyleneamide, an optionally substituted carbamate or urethane group, wherein an alkylene group or other carbon group not otherwise specified contains from 1 to 8 carbon units long (alternatively, about 2-6 carbon units long) and the alkyl group on an ester group is from 1 to 8 carbon units long, preferably up to 4 carbon units long. Various optionally substituted moieties may be substituted with 5 or more substituents, preferably no more than 3 substituents and preferably from 1 to 3 substituents.

The term “pharmaceutically acceptable salt” is used throughout the specification to describe a salt form of analogs of one or more of the compounds described herein which are presented to increase the solubility of the compound in the gastic juices of the patient's gastrointestinal tract in order to promote dissolution and the bioavailability of the compounds. Pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Additional salts include acid addition salts of amines such as, for example, HCl salts, carboxylic acid salts (malate, citratre, taurate, oxalate, etc.) and phosphate salts, among numerous others. Salt formulation is a function of the chemical formula of a given compound, as one of ordinary skill will readily understand.

The term “geometric isomer” shall be used to signify an isomer of a compound according to the present invention wherein a chemical group or atom occupies different spatial positions in relation to double bonds or in saturated ring systems having at least three members in the ring as well as in certain coordination compounds. Thus “cis” and “trans” isomers are geometric isomers as well as isomers of for example, cyclohexane and other cyclic systems. In the present invention all geometric isomers as mixtures (impure) or pure isomers are contemplated by the present invention. In preferred aspects, the present invention is directed to pure geometric isomers.

The term “optical isomer” is used to describe either of two kinds of optically active 3-dimensional isomers (stereoisomers). One kind is represented by mirror-image structures called enantiomers, which result from the presence of one or more asymmetric carbon atoms. The other kind is exemplified by diastereomers, which are not mirror images and which contain at least two asymmetric carbon atoms. Thus, such compounds have 2ₙ optical isomers, where n is the number of asymmetric carbon atoms. In the present invention all optical isomers in impure (i.e., as mixtures) or pure or substantially pure form (such as enantiomerically enriched or as separated diastereomers) are contemplated by the present invention. In certain aspects, the pure enantiomer is the preferred compound.

The term “inhibitory effective concentration” or “inhibitory effective amount” is used throughout the specification to describe concentrations or amounts of compounds according to the present invention which substantially or significantly inhibit the growth of a tumor or cancer within the context of administration to a patient.

The term “therapeutic effective amount” or “therapeutically effective amount” is used throughout the specification to describe concentrations or amounts of compounds according to the present invention which are therapeutically effective in treating tumors/cancer or the various conditions or disease states including hyperproliferative cell growth, psoriasis and related conditions, as well as arthritis and chronic inflammatory diseases, including rheumatoid arthritis and osteoarthritis, among others.

The term “preventing effective amount” is used throughout the specification to describe concentrations or amounts of compounds according to the present invention which are prophylactically effective in preventing, reducing the likelihood of contracting or delaying the onset of one or more of the disease states according to the present invention. Within the context of the present invention, a preventing effective amount is that amount, for example, which may reduce the likelihood that a precancerous lesion may become a malignant tumor or that a non-malignant tumor will become malignant. This term is subsumed under the term “effective amount”. Certain compounds according to the present invention are particularly useful as prophylactic agents because of the reduced toxicity these compounds exhibit to non-tumorigenic and/or non-cancerous cells.

The term “effective amount” shall mean an amount or concentration of a compound or composition according to the present invention which is effective within the context of its administration, which may be inhibitory, prophylactic and/or therapeutic. Compounds according to the present invention are particularly useful for providing favorable change in the disease or condition treated, whether that change is a remission, a decrease in growth or size of cancer or a tumor or other effect of the condition or disease to be treated, a favorable physiological result or a reduction in symptomology associated with the disease or condition treated.

The term “pharmaceutically acceptable carrier” refers to carrier, additive or excipient which is not unacceptably toxic to the subject to which it is administered. Pharmaceutically acceptable excipients are described at length by E. W. Martin, in “Remington's Pharmaceutical Sciences”, among other references well-known in the art.

Aspects of the present invention include compounds which have been described in detail hereinabove or to pharmaceutical compositions which comprise an effective amount of one or more compounds according to the present invention, optionally in combination with a pharmaceutically acceptable carrier, additive or excipient.

Another aspect of the present invention is directed to compounds according to the present invention which are inhibitors of farnesyl transferase of the malaria parasite Plasmodium sp. and may be used to treat malaria in veterinary (non-human) and human applications. In this aspect of the present invention, one or more of the compounds according to the present invention may be used to inhibit farnesyl transferase in a patient or subject and consequently, be useful in the treatment of malaria and other disease states or conditions.

The present invention is directed therefore to the treatment of malaria comprising administering to a patient in need of therapy an effective amount of a compound according to the present invention, optionally, in combination with a pharmaceutically acceptable additive, carrier or excipient.

The present invention is also directed to a method for treating tumors and/or cancer in a patient in need of therapy comprising administering to such a patient an effective amount of one or more compounds according to the present invention, optionally in combination with a pharmaceutically acceptable additive, carrier or excipient.

The tumors and/or cancer to be treated with compounds of the present invention include benign and malignant neoplasia, including various cancers such as, stomach, colon, rectal, liver, pancreatic, lung, breast, cervix uteri, corpus uteri, ovary, prostate, testis, bladder, renal, brain/cns, head and neck, throat, Hodgkins disease, non-Hodgkins leukemia, multiple myeloma leukemias, skin melanoma, acute lymphocytic leukemia, acute myelogenous leukemia, Ewings Sarcoma, small cell lung cancer, choriocarcinoma, rhabdomyosarcoma, Wilms Tumor, neuroblastoma, hairy cell leukemia, mouth/pharynx, oesophagus, larynx, melanoma, kidney, lymphoma, among others. Compounds according to the present invention are particularly useful in the treatment of a number of cancers, including those which are drug resistant, including multiple drug resistant cancers.

A method of treating hyperproliferative cell growth and psoriasis and related conditions using one or more of the disclosed compositions are other inventive aspects of the present invention. This method comprises administering to a patient in need of therapy an effective amount of one or more compounds according to the present invention to said patient, optionally in combination with an additive, carrier or excipient.

A method of treating arthritis and chronic inflammatory diseases, including rheumatoid arthritis and osteoarthritis, among others represent other inventive aspects of the present invention. This method comprises administering to a patient in need of therapy an effective amount of one or more compounds according to the present invention to said patient, optionally in combination with an additive, carrier or excipient.

Pharmaceutical compositions according to the present invention comprise an effective amount of one or more compounds according to the present invention optionally in combination with a pharmaceutically acceptable additive, carrier or excipient.

In another aspect, the present invention is directed to the use of one or more compounds according to the present invention in a pharmaceutically acceptable carrier, additive or excipient at a suitable dose ranging from about 0.05 to about 100 mg/kg of body weight per day, preferably within the range of about 0.1 to 50 mg/kg/day, most preferably in the range of 1 to 20 mg/kg/day. The desired dose may conveniently be presented in a single dose or as divided doses administered at appropriate intervals, for example as two, three, four or more sub-doses per day.

Ideally, the active ingredient should be administered to achieve effective peak plasma concentrations of the active compound within the range of from about 0.05 to about 5 uM. This may be achieved, for example, by the intravenous injection of about a 0.05 to 10% solution of the active ingredient, optionally in saline, or orally administered as a bolus containing about 1 mg to about 5 g, preferably about 5 mg to about 500 mg of the active ingredient, depending upon the active compound and its intended target. Desirable blood levels may be maintained by a continuous infusion to preferably provide about 0.01 to about 2.0 mg/kg/hour or by intermittent infusions containing about 0.05 to about 15 mg/kg of the active ingredient. Oral dosages, where applicable, will depend on the bioavailability of the compounds from the GI tract, as well as the pharmacokinetics of the compounds to be administered. While it is possible that, for use in therapy, a compound of the invention may be administered as the raw chemical, it is preferable to present the active ingredient as a pharmaceutical formulation, presented in combination with a pharmaceutically acceptable carrier, excipient or additive.

Pharmaceutical formulations include those suitable for oral, rectal, nasal, topical (including buccal and sub-lingual), vaginal or parenteral (including intramuscular, sub-cutaneous and intravenous) administration. Compositions according to the present invention may also be presented as a bolus, electuary or paste. Tablets and capsules for oral administration may contain conventional excipients such as binding agents, fillers, lubricants, disintegrants, or wetting agents. The tablets may be coated according to metho*ds well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. When desired, the above described formulations may be adapted to provide sustained release characteristics of the active ingredient(s) in the composition using standard methods well-known in the art.

In the pharmaceutical aspect according to the present invention, the compound(s) according to the present invention is formulated preferably in admixture with a pharmaceutically acceptable carrier. In general, it is preferable to administer the pharmaceutical composition orally, but certain formulations may be preferably administered parenterally and in particular, in intravenous or intramuscular dosage form, as well as via other parenteral routes, such as transdermal, buccal, subcutaneous, suppository or other route, including via inhalation intranasally. Oral dosage forms are preferably administered in tablet or capsule (preferably, hard or soft gelatin) form. Intravenous and intramuscular formulations are preferably administered in sterile saline. Of course, one of ordinary skill in the art may modify the formulations within the teachings of the specification to provide numerous formulations for a particular route of administration without rendering the compositions of the present invention unstable or compromising their therapeutic activity.

In particular, the modification of the present compounds to render them more soluble in water or other vehicle, for example, may be easily accomplished by minor modifications (such as salt formulation, etc.) which are well within the ordinary skill in the art. It is also well within the routineer's skill to modify the route of administration and dosage regimen of a particular compound in order to manage the pharmacokinetics of the present compounds for maximum beneficial effect to the patient.

Formulations containing the compounds of the invention may take the form of solid, semi-solid, lyophilized powder, or liquid dosage forms, such as, for example, tablets, capsules, powders, sustained-release formulations, solutions, suspensions, emulsions, sup-positories, creams, ointments, lotions, aerosols or the like, preferably in unit dosage forms suitable for simple administration of precise dosages.

The compositions typically include a conventional pharmaceutical carrier, additive or excipient and may additionally include other medicinal agents, carriers, and the like. Preferably, the composition will be about 0.05% to about 75-80% by weight of a compound or compounds of the invention, with the remainder consisting of suitable pharmaceutical additives, carriers and/or excipients. For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

Liquid compositions can be prepared by dissolving or dispersing the compounds (about 0.5% to about 20%), and optional pharmaceutical additives, in a carrier, such as, for example, aqueous saline, aqueous dextrose, glycerol, or ethanol, to form a solution or suspension. For use in oral liquid preparation, the composition may be prepared as a solution, suspension, emulsion, or syrup, being supplied either in liquid form or a dried form suitable for hydration in water or normal saline.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

An injectable composition for parenteral administration will typically contain the compound in a suitable i.v. solution, such as sterile physiological salt solution. The composition may also be formulated as a suspension in a lipid or phospholipid, in a liposomal suspension, or in an aqueous emulsion.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Methods for preparing such dosage forms are known or will be apparent to those skilled in the art; for example, see “Remington's Pharmaceutical Sciences” (17th Ed., Mack Pub. Co, 1985). The person of ordinary skill will take advantage of favorable pharmacokinetic parameters of the pro-drug forms of the present invention, where applicable, in delivering the present compounds to a patient suffering from a viral infection to maximize the intended effect of the compound.

The pharmaceutical compositions according to the invention may also contain other active ingredients such as antimicrobial agents, antiinfective agents, anti-malarial agents, anti-cancer agents or preservatives. Effective amounts or concentrations of each of the active compounds are to be included within the pharmaceutical compositions according to the present invention.

The individual components of such combinations may be administered either sequentially or simultaneously in separate or combined pharmaceutical formulations.

When one or more of the compounds according to the present invention is used in combination with a second therapeutic agent active the dose of each compound may be either the same as or differ from that when the compound is used alone. Appropriate doses will be readily appreciated by those skilled in the art.

In method aspects according to the present invention, one or more pharmaceutical compositions according to the present invention may be administered in the treatment or prevention of any disease state or condition previously mentioned. Many of these disease states or conditions are believed to elaborate through a farnseyl transferase mechanism, which may be inhibited by the compounds according to the present invention. Methods for treating conditions or disease states according to the present invention comprise administering to a patient in need thereof an effective amount of a compound according to the present invention in an amount and for a duration to treat, resolve, reduce or eliminate the condition or disease state. Conditions or disease states which may be treated using compounds according to the present invention include, for example, malaria infections tumors and/or cancer, proliferative diseases including psoriasis, genital warts and hyperproliferative keratinocyte diseases including hyperkeratosis, ichthyosis, keratoderma, lichen planus, as well as rheumatoid arthritis and osteoarthritis.

Compositions according to the present invention may be coadministered with another active compound such as anti-malarial agents, antimicrobial agents, antiinfective agents, anti-cancer agents or preservatives. When co-administered with compounds according to the present invention for the treatment of tumors, including cancer, other agents such as antimetabolites, Ara C, etoposide, doxorubicin, taxol, hydroxyurea, vincristine, cytoxan (cyclophosphamide) or mitomycin C, among numerous others, including topoisomerase I and topoisomerase II inhibitors, such as adriamycin, topotecan, campothecin and irinotecan, other agent such as gemcitabine, decitabine and agents based upon campothecin and cis-platin may be included, among numerous others. Other agents include for example, Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof. These compounds may also be included in pharmaceutical formulations or coadministered with compounds according to the present invention top produce additive or synergistic anti-cancer activity.

The individual components of such combinations as described above may be administered either sequentially or simultaneously in separate or combined pharmaceutical formulations. When one or more of the compounds according to the present invention is used in combination with a second therapeutic agent active the dose of each compound may be either the same as or differ from that when the compound is used alone. Appropriate doses will be readily appreciated by those skilled in the art.

### Chemical Synthesis

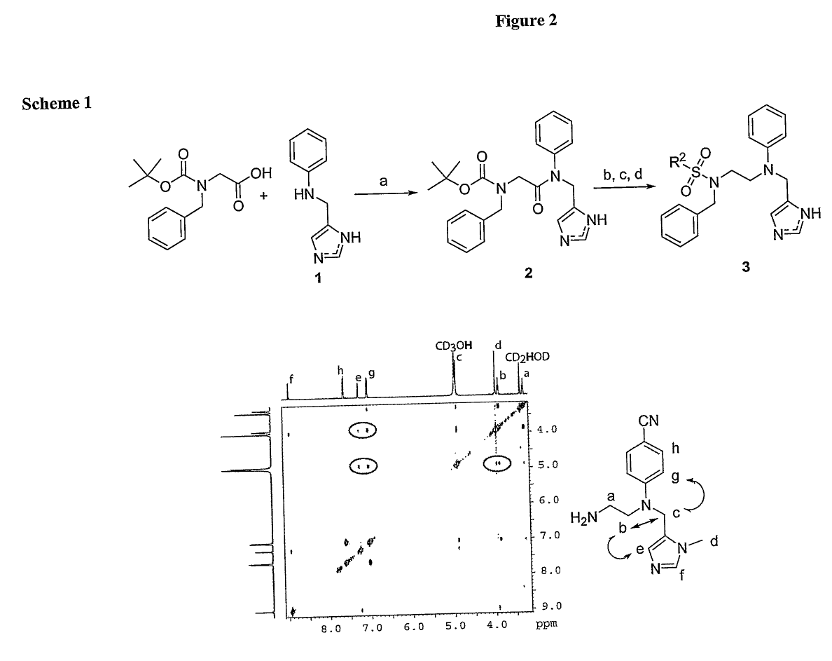

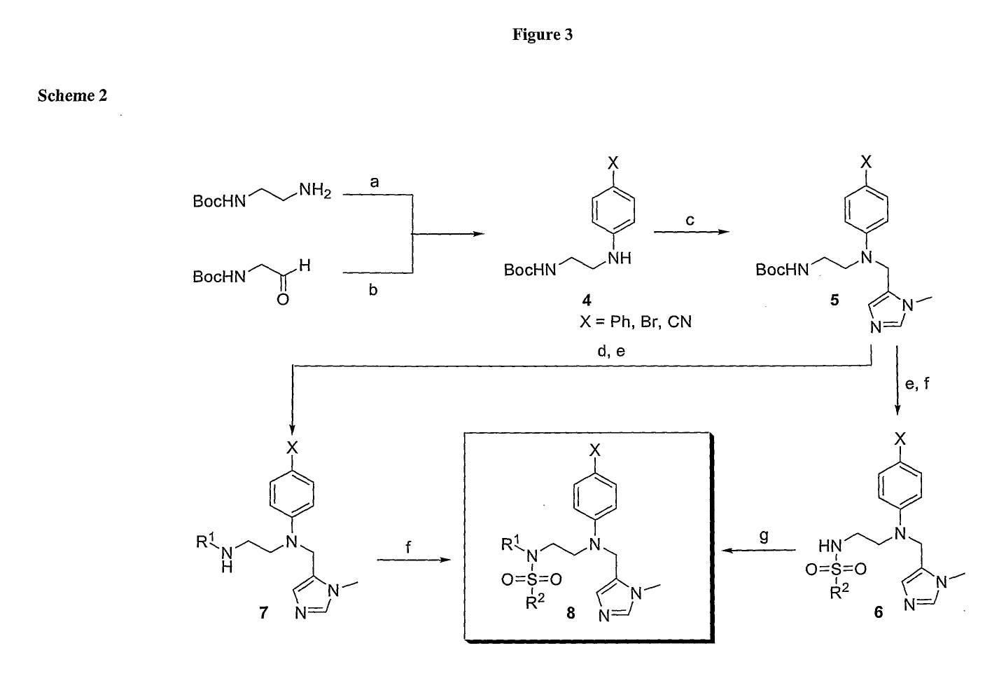

Compounds according to the present invention may be readily synthesized using methods available in the art. Pursuant to the synthetic methods which are described in FIG. 2, Scheme 1 and FIG. 3, Scheme 2. In Scheme 1, an imidazole amine is reacted with a blocked carboxylic acid compound, which intermediate compound is subsequently deblocked and then reacted with a sulfonyl compound to produce the final product 3. A large number of compounds according to the present invention may be synthesized using this general method as set forth in scheme 1. Alternatively, as set forth in scheme 2, a blocked ethylenediamine (a) or a blocked aldehyde amine (2) is reacted to form a blocked amino aniline compound 4 which is reacted with an methyl imidazole compound to produce 5, which is further reacted with a sulfonyl compound followed by reaction with an alkyl halide (alkyl bromide) to produce compound 8 or alternatively, compound 5 may be reacted with

### EXAMPLES

### Experimental

Chemistry: General Methods. ¹H and ¹³C NMR spectra were recorded on either Bruker AM-400 or AM-500 MHz spectrometers. Analysis and purification by rpHPLC were performed using either Phenomenex Luna 5μ C18(2) 250×21 mm column run at 15 mL/minute (preparative), or a Microsorb-MV 300 Å C18 250×4.6 mm column run at 1 mL/minute (analytical), using gradient mixtures of water:0.1% TFA (A) and 10:1 acetonitrile/water (B) with 0.1% TFA, and product fractions were always lyophilized to dryness. Inhibitor purity was confirmed by analytical rpHPLC using linear gradients from 100% A to 100% B with changing solvent composition of either: (I) 4.5% or (II) 1.5% per minute after an initial 2 minutes of 100% A. Mass determinations were performed using electrospray ionization on either a Varian MAT-CH-5 (HRMS) or Waters Micromass ZQ (LRMS). Solvents: DMF, THF, and CH₂Cl₂ were dried on an Innovative Technology SPS-400 dry solvent system. Methanol, TEA and DMSO were dried over calcium hydride. Molecular sieves were activated by heating to 300° C. under vacuum overnight. Flexible ligand docking was performed using GOLD,²² with ligand minimization performed within InsightII on a SGI O2.

General Procedure A (Alkylation of carbamates): Sodium hydride (60% dispersion, 1.5 equiv) was added in one portion to a solution of the carbamate (1.0 equiv) dissolved in DMF (2 mL/mmol) at 0° C. The resulting suspension was stirred for 5 minutes before addition of the alkyl halide (1.1 equiv), and stirring was then continued for a further 10 minutes. The resulting solution was diluted with EtOAc (20 mL/mmol) and washed consecutively with equal portions of 1.0 M aqueous HCl, saturated NaHCO₃, and brine. The organic phase was dried over magnesium sulfate, and the solvent was removed under vacuum. The crude reaction product was generally deprotected immediately by dissolving the crude material in TFA (1 mL/mmol) and stirring for 10 minutes. After removing the TFA under reduced pressure, the resulting oil was purified by rpHPLC to provide the product amine as the TFA salt.

General Procedure B (Alkylation of sulfonamides): The required alkyl bromide (1.1 equiv) was added in one portion to a solution of the primary sulfonamide (1.0 equiv) and Cs₂CO₃ (1.5 equiv) in DMF (5 mL/mmol), and the resulting solution stirred for two days at room temperature. Filtration and purification by rpHPLC provided the desired compound as the TFA salt.

General Procedure C (Reaction of amines with sulfonyl or acid chlorides): The required sulfonyl or acid chloride (1.2 equiv) was added in one portion to a solution of the amine (1.0 equiv) and dry TEA (5.0 equiv) in DMF (2 mL/mmol) at 0° C. The reaction was stirred for 10 minutes, diluted with acetonitrile and purified directly by rpHPLC to provide the desired sulfonamide as the TFA salt.

1-Trityl-1H-imidazole-4-carbaldehyde (9): Dry triethylamine (12.6 mL, 90.0 mmol) was added drop wise over two hours to a slurry of (1,3)-H-imidazole-4-carbaldehyde (5.0 g, 52 mmol) and trityl chloride (16.0 g, 57.0 mmol) in acetonitrile (170 mL). After complete addition of the triethylamine, the resulting solution was stirred overnight and then hexane (16.6 mL) and water (170 mL) were added. After stirring for an additional 30 minutes, the resulting solid was collected and dried overnight under vacuum to provide the title compound as a white solid (16.8 g, 96%). ¹H NMR (400 MHz, CDCl₃): δ 9.81 (s, 1H), 7.54 (s, 1H), 7.46 (s, 1H), 7.29 (m, 10H), 7.04 (m, 5H). ¹³C NMR (100 MHz, CDCl₃): δ 186.9, 141.9, 141.2, 141.0, 130.0, 129.0, 128.9, 127.2, 76.7.

3-Methyl-3H-imidazole-4-carbaldehyde trifluoromethanesulfonate (10): Methyl triflate (10.0 g, 60.1 mmol) was added drop wise over five hours to a solution of aldehyde 9 (13.5 g, 40.0 mmol) in CH₂Cl₂ (50 mL), and the resulting solution stirred over night at room temperature. The volume of solvent was then reduced under vacuum (˜30 mL), hexane (40 mL) was added, and stirring continued for a further 30 minutes at which time the crude solid of 3-methyl-1-trityl-1H-imidazole-4-carbaldehyde trifluoromethanesulfonate was collected, and washed with hexane (3×25 mL). This solid was immediately dissolved in 2:1 acetone/water (40 mL) and stirred for four hours at room temperature. The resulting suspension was filtered, the solid washed with water (30 mL), and the supernatant concentrated under vacuum. The resulting suspension was filtered, and the supernatant lyophilized to provide the title compound as a white solid (9.7 g, 93%). ¹H NMR (400 MHz, d₄-MeOH): δ 8.84 (s, 1H), 7.50 (s, 1H), 5.77 (s, 1H), 3.95 (s, 3H). ¹³C NMR (100 MHz, d₄-MeOH): δ 188.6, 148.4, 140.8, 135.3, 30.2.

(3-Methyl-3H-imidazol-4-yl)-methanol (11): 3-Methyl-3H-imidazole-4-carbaldehyde (10) (4.0 g, 15 mmol) was suspended in THF (10 mL), and the resulting solution cooled to 0° C. Lithium aluminum hydride (300 mg, 32.0 mmol) was added portion wise over 10 minutes, and the resulting suspension stirred for a further 10 minutes. Excess hydride was quenched by the addition of solid Na₂SO₄.10H₂O (˜1 g) in large portions with vigorous stirring. Additional THF was added as needed to prevent solidification of the resulting slurry. The resulting suspension was stirred for a further hour, and then filtered to remove the sulfate salts, and the solvent was removed under reduced pressure to provide the title alcohol (1.3 g, 80%). ¹H NMR (400 MHz, d₄-MeOH): δ 7.57 (s, 1H), 6.89 (s, 1H), 4.58 (s, 2H), 372 (s, 3H). ¹³C NMR (100 MHz, d₄-MeOH): δ 140.1, 132.7, 128.1, 31.9, 31.0.

5-Chloromethyl-1-methyl-1H-imidazole (12): DMF (1 drop) was added to a slowly stirred solution of (3-methyl-3H-imidazol-4-yl)-methanol (11) (1.8 g, 16 mmol) dissolved in thionyl chloride (12 mL). After 30 minutes the solvent was removed under reduced pressure, and the resulting solid triturated with diethyl ether (20 mL). The resulting semi-solid was dried over night under vacuum, and used without further purification. ¹H NMR (400 MHz, d₄-MeOH): δ 8.98 (s, 1H), 7.63 (s, 1H), 4.85 (s, 2H), 3.90 (s, 3H). ¹³C NMR (100 MHz, d₄-MeOH): δ 138.3, 132.6, 120.5, 34.5, 33.9.

Benzyl-{[(3H-imidazol-4-ylmethyl)-phenyl-carbamoyl]-methyl}-carbamic acid tert-butyl ester (2): HBTU (2.2 g, 5.8 mmol) was added in one portion to a solution of (benzyl-tert-butoxycarbonyl-amino)-acetic acid²⁷ (1.6 g, 5.8 mmol) and DIPEA (4.9 mL, 29 mmol) dissolved in DMF (300 ml), and the resulting solution stirred for 10 minutes before addition of (3H-imidazol-4-ylmethyl)-phenyl-amine (1) (1.0 g, 5.8 mmol). The reaction was stirred at room temperature for 1 hour, at which the volume of solvent was reduced (˜20 mL) under vacuum, and the resulting residue dissolved in EtOAc (500 mL), and washed successively with 1.0 M HCl (2×200 mL), saturated NaHCO₃ (2×200 mL), and brine (200 mL). The organic layer was dried over magnesium sulfate, and the solvent was removed under reduced pressure. Purification by flash column chromatography (1:4 MeOH/EtOAc) provided the title compound (1.88 g, 77%). LRMS calcd for C₂₄H₂₉N₄O₃⁺: 421.2. found 421.4. NMR consistent with proposed structure, but complicated by presence of configurational isomers at the carbamate and or amide. Full characterization is reported on deprotected and reduced product below.

N′-Benzyl-N-(3H-imidazol-4-ylmethyl)-N-phenyl-ethane-1,2-diamine (13): Carbamate 2 (500 mg, 1.20 mmol) was dissolved in TFA/water (100:1, 25 mL) and the resulting solution stirred for 20 minutes. The solvent was removed under reduced pressure and the residue triturated with ether, and dried under vacuum. The resulting viscous oil was dissolved in THF (50 mL), and LAH (190 mg, 5.00 mmol) was added in portions. After stirring for 1 hour at room temperature Na₂SO₄.10H₂0 (˜1.0 g) was added, and the resulting suspension stirred overnight. The reaction was filtered, and solvent removed under reduced pressure to afford the title compound, which was purified by rpHPLC (230 mg, 63%). LRMS calcd for C₁₉H₂₃N₄⁺: 307.2. found 307.1. ¹H NMR (400 MHz, d₄-MeOH): δ 8.61 (s, 1H), 7.33 (m, 5H), 7.19 (s, 1H), 7.15 (m, 2H), 7.01 (t, J=7.15 Hz, 1H), 6.82 (d, J=8.61 Hz, 2H), 4.53 (s, 2H), 4.15 (s, 2H), 3.62 (t, J=6.86 Hz, 2H), 3.15 (t, J=6.83 Hz, 2H). ¹³C NMR (125 MHz, CDCl₃): δ 147.3, 138.0, 134.7, 131.6, 129.9, 129.5, 129.2, 128.8, 126.7, 118.2, 113.3, 53.7, 50.2, 48.0, 46.9.

N-Benzyl-N-{2-[(3H-imidazol-4-ylmethyl)-phenyl-amino]-ethyl}-benzenesulfonamide (3a, X=H, R¹=Benzyl, R²=Phenyl, R³=H): Reaction of 13 according to procedure C, Yield 71%. ¹H NMR (400 MHz, d₄-MeOH): δ 8.71 (s, 1H), 7.79 (d, J=7.17 Hz, 2H), 7.61 (tt, J=1.17, 7.10 Hz, 1H), 7.54 (m, 2H), 7.24 (m, 5H), 7.05 (s, 1H), 7.13 (m, 2H), 6.93 (t, J=7.10 Hz, 1H), 6.79 (d, J=8.45 Hz, 2H), 4.31 (s, 2H), 4.18 (s, 2H), 3.54 (m, 2H), 3.19 (m, 2H). ¹³C NMR (125 MHz, CDCl₃): δ 148.4, 138.3, 138.0, 135.0, 131.3, 130.9, 129.7, 129.4, 129.2, 128.8, 126.5, 119.8, 116.8, 113.8, 55.3, 51.8, 48.9, 46.7. HRMS calcd for C₂₅H₂₆N₄O₂SH⁺ 447.1849. found 447.1840. Retention time for analytical rpHPLC: condition (I) 10.42, (II) 13.10 minutes.

1-Methyl-1H-imidazole-4-sulfonic acid benzyl-{2-[(3H-imidazol-4-ylmethyl)-phenyl-amino]-ethyl}-amide (3c, X=H, R¹=Benzyl, R²=4-Methyl-1H-imidazole, R³=H): Reaction of 13 according to procedure C, Yield 62%. ¹H NMR (400 MHz, d₄-MeOH): δ 8.74 (s, 1H), 7.75 (s, 1H), 7.69 (s, 1H), 7.28 (m, 5H), 7.22 (s, 1H), 7.04 (t, J=7.40 Hz, 2H), 6.64 (t, J=7.30 Hz, 1H), 6.45 (d, J=8.02 Hz, 2H), 4.40 (s, 2H), 4.22 (s, 2H), 3.73 (m, 5H), 3.30 (obscured). ¹³C NMR (100 MHz, d₄-MeOH): δ 148.6, 141.8, 138.3, 135.7, 133.5, 131.4, 130.7, 130.6, 130.2, 129.6, 127.0, 119.6, 118.4, 114.8, 55.2, 52.3, 46.8, 46.6, 34.7. HRMS calcd for C₂₃H₂₆N₆O₂SH⁺ 451.1916. found 451.1908. Retention time for analytical rpHPLC: condition (I) 10.49, (II) 12.95 minutes.

N-Benzyl-N-{2-[(3H-imidazol-4-ylmethyl)-phenyl-amino]-ethyl}-C-p-tolyl-methanesulfonamide (3d, X=H, R¹=Benzyl, R²=4-Methylbenzyl, R³=H): Reaction of 13 according to procedure C, Yield 71%. ¹H NMR (400 MHz, d₄-MeOH): δ 8.61 (s, 1H), 7.39 (m, 5H), 7.32 (s, 1H), 7.23 (d, J=7.80 Hz, 2H), 7.19 (m, 2H), 7.05 (d, J=7.78 Hz, 2H), 6.87 (d, J=8.58 Hz, 2H), 6.83 (t, J=7.33 Hz, 1H), 4.54 (s, 2H), 4.09 (s, 2H), 3.97 (s, 2H), 3.60 (t, J=6.96 Hz, 2H), 3.12 (t, J=6.95 Hz, 2H), 2.27 (s, 3H). ¹³C NMR (100 MHz, d₄-MeOH): δ 148.4, 138.5, 135.7, 132.7, 132.6, 132.4, 131.8, 131.5, 131.2, 131.1, 130.5, 121.9, 118.6, 117.7, 114.9, 58.6, 55.4, 52.8, 47.7, 45.5, 21.6. HRMS calcd for C₂₇H₃₀N₄O₂SH⁺ 475.2168. found 475.2154. Retention time for analytical rpHPLC: condition (I) 10.65, (II) 13.39 minutes.

Naphthalene-2-sulfonic acid benzyl-{2-[(3H-imidazol-4-ylmethyl)-phenyl-amino]-ethyl}-amide (3e, X=H, R¹=Benzyl, R²=2-Napthyl, R³=H): Reaction of 13 according to procedure C, Yield 65%. ¹H NMR (500 MHz, d₄-MeOH): δ 8.30 (s, 1H), 7.89 (m, 5H), 7.52 (m, 2H), 7.42 (m, 4H), 7.26 (s, 1H), 7.21 (m, 3H), 6.85 (d, J=7.04 Hz, 2H), 6.79 (t, J=7.36 Hz, 1H), 4.57 (s, 2H), 4.39 (s, 2H), 3.71 (t, J=6.86 Hz, 2H), 3.27 (t, J=6.79 Hz, 2H). ¹³C NMR (125 MHz, d₄-MeOH): δ 148.4, 135.9, 135.8, 134.3, 132.7, 131.4, 131.2, 131.1, 130.7, 130.2 (2C), 129.8, 129.2, 128.9, 128.3, 126.9, 124.5, 121.8, 118.5, 117.5, 52.9, 48.1, 46.7, 45.8. HRMS calcd for C₂₉H₂₈N₄O₂SH⁺ 497.2006. found 497.1998. Retention time for analytical rpHPLC: condition (I) 10.71, (II) 12.93 minutes.

Quinoline-8-sulfonic acid benzyl-{2-[(3H-imidazol-4-ylmethyl)-phenyl-amino]-ethyl}-amide (3f, X=H, R¹=Benzyl, R²=8-Quinoline, R³=H): Reaction of 13 according to procedure C, Yield 70%. ¹H NMR (400 MHz, d₄-MeOH): δ 8.79 (dd, J=1.45, 4.26 Hz, 1H), 8.63 (dd, J=1.38, 7.48 Hz, 1H), 8.39 (dd, J=1.75, 8.41 Hz, 1H), 8.32 (dd, J=1.36, 8.26, 1H), 8.21 (d, J=1.25 Hz, 1H), 7.77 (t, J=7.61 Hz, 1H), 7.57 (dd, J=4.29, 8.40 Hz, 1H), 7.43 (m, 5H), 7.20 (s, 1H), 6.99 (dd, J=7.38, 8.80 Hz, 2H), 6.61 (m, 3H), 4.32 (s, 2H), 4.21 (s, 2H), 3.74 (t, J=5.53 Hz, 2H), 3.28 (t, J=5.52 Hz, 2H). ¹³C NMR (100 MHz, d₄-MeOH): δ 155.5, 150.86, 146.8, 144.3, 143.1, 140.8, 140.5, 137.5, 136.7, 135.3, 133.5, 133.3, 133.0, 132.9, 132.7, 129.3, 126.7, 122.0, 119.9, 117.1, 54.8, 53.0, 52.0, 49.7. HRMS calcd for C₂₈H₂₇N₅O₂SH⁺ 498.1964. found 498.1956. Retention time for analytical rpHPLC: condition (I) 10.62, (II) 13.43 minutes.

5-Dimethylamino-naphthalene-1-sulfonic acid benzyl-{2-[(3H-imidazol-4-ylmethyl)-phenyl-amino]-ethyl}-amide (3g, X=H, R¹=Benzyl, R²=Dimethylaminonapthalene, R³=H): Reaction of 13 according to procedure C, Yield 63%. ¹H NMR (400 MHz, d₄-MeOH): δ 8.68 (dd, J=0.85, 8.51 Hz, 1H), 8.43 (dd, J=1.20, 7.49 Hz, 1H), 8.16 (d, J=8.69 Hz, 1H), 7.99 (d, J=1.37, 1H), 7.65 (dd, J=7.53, 8.51 Hz, 1H), 7.54 (dd, J=7.70, 8.62, Hz, 1H), 7.53 (s, 1H), 7.36 (m, 5H), 7.26 (d, J=7.67 Hz, 1H), 6.99 (dd, J=7.36, 8.78 Hz, 2H), 6.63 (t, J=7.30, 1H), 6.59 (d, J=7.96 Hz, 2H), 4.30 (s, 2H), 4.17 (s, 2H), 3.72 (t, J=5.24 Hz, 2H), 3.24 (obscured), 2.83 (s, 6H). ¹³C NMR (100 MHz, d₄-MeOH): δ 154.3, 148.6, 143.3, 139.0, 135.0, 134.0, 133.1, 132.8, 131.6, 131.4, 131.3, 131.1, 130.9, 130.7, 130.6, 125.0, 120.0, 118.7, 117.6, 117.2, 115.1, 52.6, 51.0, 48.8, 47.4, 46.1. HRMS calcd for C₃₁H₃₃N₅O₂SH⁺ 540.2428. found 540.2419. Retention time for analytical rpHPLC: condition (I) 10.70, (II) 13.51 minutes.

[2-(4-Bromophenylamino)-ethyl]-carbamic acid tert-butyl ester (4b, X=Br): A solution of 4-bromoaniline (6.5 g, 37 mmol), (2-oxoethyl)-carbamic acid tert-butyl ester (2.0 g, 12 mmol) and acetic acid (790 μL, 12.4 mmol) in dry methanol (20 mL) with dry 3 molecular sieves (1.0 g) was stirred under nitrogen for 20 minutes. Sodium cyanoborohydride (0.79 g, 12 mmol) was added in one portion, and the resulting solution stirred for a further hour under nitrogen, at which time EtOAc (300 mL) was added, and the organic phase washed consecutively with 1.0 M aqueous HCl (1×100 mL), saturated NaHCO₃ (2×100 mL), and brine (1×100 mL). The organic phase was dried over magnesium sulfate, and the solvent was removed under vacuum to provide the title compound as a viscous yellow oil (3.1 g, 79%) after flash column chromatography (1:4 EtOAc/Hexane). ¹H NMR (400 MHz, CDCl₃): δ 7.14 (d, J=8.88 Hz, 2H), 6.38 (d, J=8.89 Hz, 2H), 4.88 (br s, 1H), 3.26 (br s, 2H), 3.09 (br t, J=5.94 Hz, 2H), 1.37 (s, 9H). ¹³C NMR (100 MHz, CDCl₃): δ 157.0, 147.5, 132.6, 114.6, 109.2, 80.1, 44.5, 40.3, 29.0.

{2-[(4-Bromo-phenyl)-(3H-imidazol-4-ylmethyl)-amino]-ethyl}-carbamic acid tert-butyl ester (5b-H, X=Br, R³=H): A solution of aniline 4b (0.50 g, 1.6 mmol), and 3H-imidazole-4-carbaldehyde (0.30 g, 3.2 mmol) in dry methanol (5.0 mL) with dry 3 molecular sieves (0.20 g) was stirred under nitrogen for 20 minutes. Sodium cyanoborohydride (0.20 g, 3.2 mmol) was added in one portion, and the resulting solution was stirred overnight under nitrogen at 50° C. The resulting solution was diluted with EtOAc (100 mL), and then washed consecutively with 1.0 M aqueous HCl (1×100 mL), saturated NaHCO₃ (2×100 mL), and brine (1×100 mL). The organic phase was dried over magnesium sulfate, and the solvent was removed under vacuum to provide the title compound as a viscous yellow oil which was purified by flash column chromatography (10:1 CH₂Cl₂/MeOH) (0.21 g, 33%). ¹H NMR (400 MHz, CDCl₃): δ 7.46 (s, 1H), 7.16 (d, J=9.02 Hz, 2H), 6.71 (s, 1H), 6.60 (d, J=9.04 Hz, 2H), 4.36 (s, 2H), 3.40 (t, J=6.31 Hz, 2H), 3.21 (t, J=6.30 Hz, 2H), 1.35 (s, 9H). ¹³C NMR (100 MHz, CDCl₃): δ 157.0, 147.7, 135.4, 132.2 (2C), 117.1, 115.0, 109.2, 80.0, 51.8, 48.6, 38.8, 28.7.

1-Methyl-1H-imidazole-4-sulfonic acid {2-[(4-bromo-phenyl)-(3H-imidazol-4-ylmethyl)-amino]-ethyl}-amide (6b, X=Br, R²=4-Methyl-1H-imidazole, R³=H): Reaction of 5b-H according to procedure C after deprotection of Boc group with TFA. Yield 71%. ¹H NMR (400 MHz, d₄-MeOH): δ 8.76 (s, 1H), 7.63 (s, 1H), 7.54 (s, 1H), 7.30 (s, 1H), 7.24 (d, J=8.86 Hz, 2H), 6.63 (d, J=8.91 Hz, 2H), 4.59 (s, 2H), 3.65 (s, 3H), 3.48 (t, J=6.36 Hz, 2H), 3.08 (t, J=6.17 Hz, 2H). ¹³C NMR (100 MHz, d₄-MeOH): δ 148.0, 141.6, 141.0, 135.8, 133.4, 133.3, 126.3, 118.5, 117.0, 111.5, 52.9, 44.7, 41.6, 34.7.

1-Methyl-1H-imidazole-4-sulfonic acid benzyl-{2-[(4-bromo-phenyl)-(3H-imidazol-4-ylmethyl)-amino]-ethyl}-amide (8a, X=Br, R¹=Benzyl, R²=4-Methyl-1H-imidazole, R³=H): Reaction of 6b according to procedure B, Yield 65%. ¹H NMR (500 MHz, d₆-DMSO): δ 8.83 (s, 1H), 7.83 (s, 1H), 7.80 (s, 1H), 7.38 (m, 5H), 7.23 (s, 1H), 7.22 (d, J=9.01 Hz, 2H), 6.46 (d, J=9.05 Hz, 2H), 4.46 (s, 2H), 4.32 (s, 2H), 3.82 (s, 3H), 3.37 (m, 4H). ¹³C NMR (125 MHz, d₆-DMSO): δ 147.6, 141.5, 139.4, 138.0, 135.6, 133.1, 130.5, 129.9, 129.5, 129.3, 126.7, 118.2, 116.1, 110.8. HRMS calcd for C₂₃H₂₆BrN₆O₂S⁺ 529.1021. found 529.1036. Retention time for analytical rpHPLC: condition (I) 13.36, (II) 21.53 minutes.

{2-[(4-Bromophenyl)-(3-methyl-3H-imidazol-4-ylmethyl)-amino]-ethyl}-carbamic acid tert-butyl ester (5b-Me, X=Br, R³=CH₃): Lithium diisopropylamide (2.0 M, 4.9 mL, 9.8 mmol) was added dropwise to a solution of 4b (1.01 g, 3.23 mmol) in dry THF (40 mL) at −78° C. and the resulting orange solution was stirred for 1.5 hour at −78° C. under nitrogen. In a separate flask sodium hydride (60%, 194 mg, 4.85 mmol) was added to a solution of 5-chloromethyl-1-methyl-1H-imidazole.HCl (594 mg, 3.55 mmol) in dry THF (15 mL) at 0° C. The suspension of sodium chloride and imidazole was added to the dianion of 4b via cannula under nitrogen, and the resulting solution stirred for 1 h at −78° C. The reaction was quenched by addition of brine (1 mL) and THF was evaporated. After diluting with EtOAc (200 mL), the organic layer was washed consecutively with water (3×50 mL) and brine (1×50 mL). The organic phase was dried over sodium sulfate, and the solvent was removed under vacuum. Purification by flash column chromatography (1:7:292 NH₄OH/MeOH/CH₂Cl₂) provided the title compound as a white solid (600 mg, 98% b.r.s.m). ¹H NMR (400 MHz, d₄-MeOH): δ 8.78 (s, 1H), 7.22 (d, J=9.01, Hz, 2H), 7.17 (s, 1H), 6.74 (d, J=9.14 Hz, 2H), 4.58 (s, 2H), 3.79 (s, 3H), 3.40 (t, J=6.87 Hz, 2H), 3.14 (t, J=6.70 Hz, 2H), 1.32 (s, 9H). ¹³C NMR (125 MHz, CDCl₃): δ 158.9, 148.5, 137.9, 134.0, 133.6, 119.6, 118.5, 111.5, 80.6, 51.9, 46.7, 39.1, 34.6, 29.1.

N′-Benzyl-N-(4-bromophenyl)-N-(3-methyl-3H-imidazol-4-ylmethyl)-ethane-1,2-diamine (7b, X=Br, R¹=Benzyl, R³=Methyl): Reaction of 5b-Me according to procedure A, Yield 73%. ¹H NMR (400 MHz, d₄-MeOH): δ 8.75 (s, 1H), 7.35 (s, 5H), 7.26 (d, J=9.02 Hz, 2H), 7.12 (s, 1H), 6.75 (d, J=9.06 Hz, 2H), 4.57 (s, 2H), 4.14 (s, 2H), 3.74 (s, 3H), 3.60 (t, J=7.11 Hz, 2H), 3.17 (t, J=7.20 Hz, 2H). ¹³C NMR (100 MHz, d₄-MeOH): δ 147.5, 138.2, 133.9, 133.3, 132.7, 131.4, 131.2, 130.8, 119.8, 118.5, 113.5, 53.0, 48.5, 46.6, 45.3, 34.6.

1-Methyl-1H-imidazole-4-sulfonic acid benzyl-{2-[(4-bromo-phenyl)-(3-methyl-3H-imidazol-4-ylmethyl)-amino]-ethyl}-amide (8b, X=Br, R¹=Benzyl, R²=4-Methyl-1H-imidazole, R³=Methyl): Reaction of 7b according to procedure C, Yield 65%. ¹H NMR (400 MHz, d₄-MeOH): δ 8.71 (s, 1H), 7.70 (s, 1H), 7.65 (s, 1H), 7.23 (m, 5H), 7.17 (s, 1H), 7.07 (d, J=9.10 Hz, 2H), 6.30 (d, J=9.15 Hz, 2H), 4.33 (s, 2H), 4.16 (s, 2H), 3.69 (s, 3H), 3.21 (obscured, 4H). ¹³C NMR (100 MHz, d₄-MeOH): δ 147.8, 141.9, 139.6, 138.2, 135.9, 133.4, 133.0, 130.6, 130.2, 129.7, 127.1, 118.5, 116.4, 111.2, 55.3, 52.7, 46.7, 46.6, 34.7. HRMS calcd for C₂₄H₂₇BrN₆O₂SH⁺ 543.1178. found 543.1186. Retention time for analytical rpHPLC: condition (I) 14.26, (II) 29.12 minutes.

[2-(Biphenyl-4-ylamino)-ethyl]-carbamic acid tert-butyl ester (4a, X=Ph): Prepared as described for 4b, Yield 52%. ¹H NMR (400 MHz, d₄-MeOH): δ 7.54-7.51 (m, 3H), 7.46 (d, J=8.83 Hz, 2H), 7.35 (t, J=7.70 Hz, 2H), 7.21 (t, J=7.33 Hz, 1H), 6.96 (d, J=8.83 Hz, 2H), 6.78 (s, 1H), 4.52 (s, 2H), 3.58 (s, 3H), 3.41 (t, J=6.75 Hz, 2H), 3.17 (t, J=6.75 Hz, 2H), 1.41 (s, 9H). ¹³C NMR (100 MHz, d₄-MeOH): [symbol] 159.1, 149.6, 142.8, 131.0, 129.8, 128.7, 127.1, 127.0, 114.2, 80.3, 44.8, 41.0, 28.9.

{2-[Biphenyl-4-yl-(3-methyl-3H-imidazol-4-ylmethyl)-amino]-ethyl}-carbamic acid tert-butyl ester (5a, X=Ph, R³=Methyl): Prepared as described to 5c, Yield 36%. ¹H NMR (400 MHz, d₄-MeOH): [symbol] 7.54-7.51 (m, 3H), 7.46 (d, J=8.83 Hz, 2H), 7.35 (t, J=7.70, 2H), 7.21 (t, J=7.33 Hz, 1H), 6.96 (d, J=8.83, 2H), 6.78 (s, 1H), 4.52 (s, 2H), 3.58 (s, 3H), 3.41 (t, J=6.75 Hz, 2H), 3.17 (t, J=6.75, 2H). ¹³C NMR (100 MHz, d₄-MeOH): [symbol] 158.7, 149.3, 142.5, 140.1, 131.8, 131.7, 129.9, 128.8, 128.7, 127.3, 127.2, 115.4, 80.3, 50.7, 45.7, 38.9, 32.3, 29.0.

N-Biphenyl-4-yl-N′-(2-methyl-benzyl)-N-(3-methyl-3H-imidazol-4-ylmethyl)-ethane-1,2-diamine (7a, X=Phenyl, R¹=o-Methylbenzyl, R³=Methyl): Reaction of 5a according to procedure A, Yield 43%. ¹H NMR (400 MHz, d₄-MeOH): δ 8.87 (s, 1H), 7.57-7.54 (m, 4H), 7.43-7.37 (m, 4H), 7.32-7.25 (m, 4H), 7.05 (d, J=8.8 Hz, 2H), 4.76 (s, 2H), 4.30 (s, 2H), 3.87 (s, 3H), 3.81 (t, J=7.3 Hz, 2H), 3.38 (t, J=7.3 Hz, 2H), 2.41 (s, 3H). ¹³C NMR (100 MHz, d₄-MeOH): [symbol] 147.3, 141.7, 138.8, 137.6, 134.1, 133.2, 132.2, 131.3, 130.8, 129.8, 129.1, 127.8, 127.7 127.3, 119.3, 116.8, 114.4, 49.8, 48.0, 46.2, 45.4, 34.3, 19.2.

1-Methyl-1H-imidazole-4-sulfonic acid {2-[biphenyl-4-yl-(3-methyl-3H-imidazol-4-ylmethyl)-amino]-ethyl}-(2-methyl-benzyl)-amide (8c, X=Phenyl, R¹=O-Methylbenzyl, R²=1-Methyl-1H-imidazole, R³=Methyl): Reaction of 7c according to procedure C, Yield 44%. ¹H NMR (400 MHz, d₄-MeOH): δ 8.84 (s, 1H), 7.82 (s, 1H), 7.79 (s, 1H), 7.50 (d, J=7.6 Hz, 2H), 7.39-7.20 (m, 9H), 7.16 (s, 1H), 6.52 (d, J=8.8 Hz, 2H), 4.41 (s, 2H), 4.28 (s, 2H), 3.81 (s, 3H), 3.80 (s, 3H), 3.29-3.19 (m, 4H), 2.38 (s, 3H). ¹³C NMR (100 MHz, d₄-MeOH): [symbol] 147.6, 142.0, 141.5, 139.6, 138.6, 137.5, 135.0, 133.5, 132.2, 132.1, 132.0, 129.8, 129.7, 128.8, 127.4, 127.2, 126.8, 119.4, 114.6, 53.9, 51.4, 45.8, 44.9, 34.4, 34.2, 19.4. HRMS (ESI): m/z calcd for C₃₁H₃₄N₆O₂SH⁺ 555.2542. found 555.2533. Retention time for analytical rpHPLC: condition (I) 14.62, (II) 30.89 minutes.

[2-(4-Cyanophenylamino)-ethyl]-carbamic acid tert-butyl ester (4c, X=CN): Freshly distilled TEA (8.9 mL, 90 mmol) was added to a solution of (2-aminoethyl)-carbamic acid tert-butyl ester (5.0 g, 30 mmol) and 4-fluorobenzonitrile (3.6 g, 30 mmol) in dry DMSO (250 mL), and the resulting solution heated to 120° C. for two days. A distillation head and condenser was fitted to the reaction, and the volume of solvent was reduced to ˜20 mL under reduced pressure. The resulting solution was dissolved in EtOAc (300 mL) and washed consecutively with 1.0 M aqueous HCl (1×100 mL), saturated NaHCO₃ (2×100 mL), and brine (1×100 mL). The organic phase was dried over magnesium sulfate, and the solvent was removed under vacuum to provide the title compound as a yellow solid (7.0 g, 89%) after flash column chromatography (1:1 EtOAc/Hexane). ¹H NMR (400 MHz, CDCl₃): δ 7.43 (d, J=8.64 Hz, 2H), 6.58 (d, J=8.62 Hz, 2H), 3.41 (br s, 2H), 3.29 (br t, J=5.67 Hz, 2H), 1.47 (s, 9H). ¹³C NMR (100 MHz, CDCl₃): δ 156.3, 147.7, 134.1, 117.2, 112.5, 110.1, 79.6, 46.6, 40.5, 28.7.

{2-[(4-Cyanophenyl)-(3-methyl-3H-imidazol-4-ylmethyl)-amino]-ethyl}-carbamic acid tert-butyl ester (5c, X=CN, R³=Methyl): The title compound was prepared as described for 5b and purified by flash column chromatography (1:7:292 NH₄OH/MeOH/CH₂Cl₂, 85% b.r.s.m). ¹H NMR (500 MHz, d₄-MeOH): δ 8.82 (s, 1H), 7.42 (d, J=8.97 Hz, 2H), 7.15 (s, 1H), 6.88 (d, J=9.00 Hz, 2H), 4.70 (s, 2H), 3.82 (s, 3H), 3.51 (t, J=6.69 Hz, 2H), 3.20 (obscured t, J=6.70 Hz, 2H), 1.31 (s, 9H). ¹³C NMR (500 MHz, CDCl₃): δ 156.5, 151.4, 139.4, 134.0, 129.1, 127.2, 120.5, 112.9, 99.4, 80.0, 49.5, 44.9, 38.1, 32.1, 28.7.

4-[(2-Benzylaminoethyl)-(3-methyl-3H-imidazol-4-ylmethyl)-amino]-benzonitrile (7d, X=CN, R¹=Benzyl, R³=Methyl): Reaction of 5c according to procedure A, Yield 65%. ¹H NMR (400 MHz, d₄-MeOH): δ 8.88 (s, 1H), 7.55 (d, J=8.73 Hz, 2H), 7.45 (s, 5H), 7.19 (s, 1H), 6.95 (d, J=8.87 Hz, 2H), 4.82 (s, 2H), 4.20 (s, 2H), 3.87 (s, 3H), 3.34 (br s, 2H), 3.24 (br s, 2H). ¹³C NMR (100 MHz, d₄-MeOH): δ. 151.7, 138.2, 134.2, 133.4, 132.5, 131.3, 131.1, 130.8, 122.0, 116.2, 114.6, 102.0, 53.2, 47.9, 46.0, 45.2, 34.7.

1-Methyl-1H-imidazole-4-sulfonic acid benzyl-{2-[(4-cyano-phenyl)-(3-methyl-3H-imidazol-4-ylmethyl)-amino]-ethyl}-amide (8d, X=CN, R¹=Benzyl, R²=4-Methyl-1H-imidazole, R³=Methyl): Reaction of 7d according to procedure C, Yield 66%. ¹H NMR (400 MHz, d₄-MeOH): δ 8.79 (s, 1H), 7.73 (s, 1H), 7.69 (s, 1H), 7.32 (d, J=9.07 Hz, 2H), 7.25 (m, 5H), 7.06 (s, 1H), 6.48 (d, J=8.90 Hz, 2H), 4.44 (s, 2H), 4.17 (s, 2H), 3.73 (s, 3H), 3.72 (s, 3H), 3.35 (m, 2H), 3.27 (m, 2H). ¹³C NMR (100 MHz, d₄-MeOH): δ 151.9, 141.9, 139.3, 138.2, 135.1, 133.1, 130.8, 130.2, 129.7, 127.2, 121.2, 119.5, 114.0, 100.6, 55.6, 51.5, 46.8, 45.3, 34.7, 34.6. HRMS calcd for C₂₅H₂₈N₇O₂S⁺: 490.2025. found 490.2028. Retention time for analytical rpHPLC: condition (I) 12.77, (II) 24.84 minutes.

N-Benzyl-N-{2-[(4-cyano-phenyl)-(3-methyl-3H-imidazol-4-ylmethyl)-amino]-ethyl}-benzenesulfonamide (8e, X=CN, R¹=Benzyl, R²=Phenyl, R³=Methyl): Reaction of 7d according to procedure C, Yield 67%. ¹H NMR (500 MHz, d₄-MeOH): δ 8.78 (s, 1H), 7.81 (s, 1H), 7.80 (s, 1H), 7.60 (t, J=7.40 Hz, 1H), 7.54 (m, 2H), 7.33 (d, J=9.08 Hz, 2H), 7.22 (m, 5H), 7.03 (s, 1H), 6.50 (d, J=9.13 Hz, 2H), 4.40 (s, 2H), 4.18 (s, 2H), 3.72 (s, 3H), 3.33 (m, 2H), 3.15 (m, 2H). ¹³C NMR (125 MHz, d₄-MeOH): δ 151.8, 140.0, 138.2, 138.0, 135.1, 134.8, 133.1, 131.0, 130.8, 130.3, 129.8, 128.9, 119.5, 114.1, 101.4, 55.7, 51.5, 46.8, 45.4, 34.6. HRMS calcd for C₂₇H₂₇N₅O₂SH⁺ 486.1958. found 486.1963. Retention time for analytical rpHPLC: condition (I) 13.35, (II) 26.31 minutes.

Thiophene-2-sulfonic acid benzyl-{2-[(4-cyano-phenyl)-(3-methyl-3H-imidazol-4-ylmethyl)-amino]-ethyl}-amide (8f, X=CN, R¹=Benzyl, R²=2-Thiophene, R³=Methyl): Reaction of 7d according to procedure C, Yield 59%. ¹H NMR (500 MHz, d₄-MeOH): δ 8.79 (s, 1H), 7.79 (dd, J=1.17, 5.01 Hz, 1H), 7.61 (dd, J=1.19, 3.70 Hz, 1H), 7.34 (d, J=8.97 Hz, 2H), 7.25 (m, 5H), 7.16 (dd, J=3.82, 4.97 Hz, 1H), 7.04 (s, 1H), 6.51 (d, J=8.98 Hz, 2H), 4.39 (s, 2H), 4.19 (s, 2H), 3.72 (s, 3H), 3.39 (m, 2H), 3.17 (m, 2H). ¹³C NMR (125 MHz, d₄-MeOH): δ 151.8, 139.6, 138.2, 137.8, 135.1, 134.4, 134.3, 133.0, 130.8, 130.3, 129.9, 129.5, 121.1, 129.6, 114.1, 100.8, 55.9, 51.5, 47.1, 45.4, 34.6. HRMS calcd for C₂₅H₂₆N₅O₂S₂⁺: 492.1528. found 492.1515. Retention time for analytical rpHPLC: condition (I) 13.91, (II) 22.82 minutes.

Pyridine-2-sulfonic acid benzyl-{2-[(4-cyano-phenyl)-(3-methyl-3H-imidazol-4-ylmethyl)-amino]-ethyl}-amide (8g, X=CN, R¹=Benzyl, R²=2-Pyridyl, R³=Methyl): Reaction of 7d according to procedure C, Yield 57%. ¹H NMR (400 MHz, d₆-DMSO): δ 8.91 (s, 1H), 8.66 (d, J=5.41 Hz, 1H), 8.00 (td, J=1.37, 7.73 Hz, 1H), 7.89 (d, J=7.78 Hz, 1H), 7.63 (dd, J=4.69, 7.57 Hz, 1H), 7.41 (d, J=8.82 Hz, 2H), 7.22 (m, 5H), 7.13 (s, 1H), 6.55 (d, J=8.96 Hz, 2H), 4.44 (s, 2H), 4.36 (s, 2H), 3.65 (s, 3H), 3.29 (m, 4H). ¹³C NMR (100 MHz, d₄-MeOH): δ 157.1, 150.7, 150.3, 139.4, 137.0, 136.8, 133.7, 130.7, 128.9 (2C), 128.2, 127.9, 122.8, 120.3, 118.0, 112.7, 98.1, 53.3, 49.4, 45.6, 44.0, 33.7. HRMS calcd for C₂₆H₂₇N₆O₂S⁺: 487.1916. found 487.1903. Retention time for analytical rpHPLC: condition (I) 13.47, (II) 21.41 minutes.

Quinoline-8-sulfonic acid benzyl-{2-[(4-cyano-phenyl)-(3-methyl-3H-imidazol-4-ylmethyl)-amino]-ethyl}-amide (8h, X=CN, R¹=Benzyl, R²=8-Quinoline, R³=Methyl): Reaction of 7d according to procedure C, Yield 63%. ¹H NMR (400 MHz, d₄-MeOH): δ 8.92 (dd, J=1.72, 4.20 Hz, 1H), 8.79 (s, 1H), 8.41 (dd, J=1.32, 7.39 Hz, 1H), 8.37 (dd, J=1.67, 8.40 Hz, 1H), 8.15 (dd, J=1.19, 8.21 Hz, 1H), 7.64 (t, J=7.67 Hz, 1H), 7.56 (dd, J=4.23, 8.35 Hz, 1H), 7.35 (d, J=9.04 Hz, 2H), 7.18 (m, 5H), 7.06 (s, 1H), 6.57 (d, J=9.07 Hz, 2H), 4.46 (s, 2H), 4.40 (s, 2H), 3.74 (s, 3H), 3.64 (m, 2H), 3.53 (m, 2H). ¹³C NMR (100 MHz, d₄-MeOH): δ 152.9, 152.0, 145.5, 138.8, 138.6, 138.2, 135.8, 135.1, 134.8, 133.1, 131.1, 130.5, 130.1, 129.6, 127.2, 124.0, 121.2, 119.5, 114.1, 100.6, 55.5, 31.6, 47.3, 45.4, 34.6. HRMS calcd for C₃₀H₂₉N₆O₂S⁺: 537.2073. found 537.2073. Retention time for analytical rpHPLC: condition (I) 14.34, (II) 24.08 minutes.

5-Dimethylamino-naphthalene-1-sulfonic acid benzyl-{2-[(4-cyano-phenyl)-(3-methyl-3H-imidazol-4-ylmethyl)-amino]-ethyl}-amide (8i, X=CN, R¹=Benzyl, R²=5-Dimethylamino-naphthalene, R³=Methyl): Reaction of 7d according to procedure C, Yield 64%. ¹H NMR (400 MHz, d₄-MeOH): δ 8.78 (s, 1H), 8.58 (d, J=8.54 Hz, 1H), 8.28 (d, J=8.66 Hz, 1H), 8.09 (d, J=7.34 Hz, 1H), 7.53 (d, J=7.30 Hz, 1H), 7.49 (d, J=7.16 Hz, 1H), 7.32 (d, J=9.04 Hz, 2H), 7.23 (d, J=7.39 Hz, 1H), 7.18 (m, 5H), 6.99 (s, 1H), 6.48 (d, J=9.09 Hz, 2H), 4.41 (s, 2H), 4.37 (s, 2H), 3.71 (s, 3H), 3.32 (m, 4H), 2.82 (s, 6H). ¹³C NMR (100 MHz, d₄-MeOH): δ 153.5, 151.2, 138.2, 138.0, 136.1, 135.1, 133.5, 133.1, 132.2, 131.8, 131.1, 130.6, 130.2, 129.8, 129.7, 124.9, 121.2, 119.4, 117.1, 114.1, 100.9, 54.2, 50.7, 46.2, 45.7, 45.6, 34.6. HRMS calcd for C₃₃H₃₅N₆O₂S⁺: 579.2542. found 579.2546. Retention time for analytical rpHPLC: condition (I) 14.67, (II) 30.30 minutes.

N-Benzyl-N-{2-[(4-cyano-phenyl)-(3-methyl-3H-imidazol-4-ylmethyl)-amino]-ethyl}-acetamide (8j, X=CN, R¹=Benzyl, R²=Acetyl, R³=Methyl): Reaction of 7d according to procedure C, Yield 74%. ¹H NMR (400 MHz, d₄-MeOH): δ 8.08 (s, 1H), 6.71 (d, J=9.08 Hz, 2H), 6.59 (br t, J=7.15 Hz, 2H), 6.54 (br t, J=7.32 Hz, 1H), 6.47 (br t, J=7.12 Hz, 2H), 6.40 (br s, 1H), 6.10 (d, J=9.11 Hz, 2H), 3.92 (s, 2H), 3.84 (s, 2H), 3.07 (s, 3H), 2.77 (m, 2H), 2.52 (s, 2H). ¹³C NMR (100 MHz, d₄-MeOH): δ 172.5, 130.1, 136.2, 136.0, 132.9, 131.2, 128.3, 127.2, 126.4, 119.0, 117.2, 112.1, 98.5, 52.5, 43.2, 42.9, 32.5, 32.4, 19.9. HRMS calcd for C₂₃H₂₆N₅O⁺: 388.2137. found 388.2131. Retention time for analytical rpHPLC: condition (I) 12.74, (II) 18.88 minutes.

N-Benzyl-N-{2-[(4-cyano-phenyl)-(3-methyl-3H-imidazol-4-ylmethyl)-amino]-ethyl}-isobutyramide (8k, X=CN, R¹=Benzyl, R²=ⁱPropylcarbonyl, R³=Methyl): Reaction of 7d according to procedure C, Yield 71%. ¹H NMR (400 MHz, d₄-MeOH): δ 8.09 (s, 1H), 6.70 (d, J=9.05 Hz, 2H), 6.61 (br t, J=7.21 Hz, 2H), 6.54 (br t, J=7.36 Hz, 1H), 6.45 (br t, J=7.23 Hz, 2H), 6.39 (br s, 1H), 6.15 (d, J=9.05 Hz, 2H), 3.93 (s, 2H), 3.91 (s, 2H), 3.08 (s, 3H), 2.79 (m, 2H), 2.52 (s, 2H), 2.12 (m, 1H), 0.26 (d, J=6.60 Hz, 6H). ¹³C NMR (100 MHz, d₄-MeOH): δ 178.4, 149.7, 136.1, 135.5, 132.5, 130.7, 127.7, 226.6, 125.6, 118.5, 416-5, 111.5, 97.9, 30.9, 46.1, 42.8, 42.7, 32.0, 29.3, 17.6. HRMS calcd for C₂₅H₃₀N₅O⁺: 416.2450. found 416.2436. Retention time for analytical rpHPLC: condition (I) 13.24, (II) 20.24 minutes.

4-[(2-Allylamino-ethyl)-(3-methyl-3H-imidazol-4-ylmethyl)-amino]-benzonitrile (7l, X=CN, R¹=Allyl, R³=Methyl): Reaction of 5c according to procedure A, Yield 65%. ¹H NMR (400 MHz, d₄-MeOH): [symbol] 8.92 (s, 1H), 7.56 (d, J=8.95 Hz, 2H), 7.22 (s, 1H), 7.00 (d, J=8.95 Hz, 2H), 5.97 (m, 1H), 5.53 (m, 1H), 5.36 (m, 1H), 4.81 (s, 2H), 3.91 (s, 3H), 3.84 (t, J=7.25 Hz, 2H), 3.72 (d, J=6.80 Hz, 2H), 3.31 (t, J=7.25 Hz, 2H). ¹³C NMR (100 MHz, d₄-MeOH): [symbol] 151.4, 137.9, 134.9, 129.1, 124.4, 118.8, 114.3, 101.4, 52.8, 51.2, 47.5, 45.7, 44.5, 37.4, 34.2.

4-[[2-(2-Methyl-allylamino)-ethyl]-(3-methyl-3H-imidazol-4-ylmethyl)-amino]-benzonitrile (7m, X=CN, R¹=2-Methyl-allyl, R³=Methyl): Reaction of 5c according to procedure A, Yield 60%. ¹H NMR (400 MHz, d₄-MeOH): δ 8.93 (s, 1H), 7.56 (d, J=9.0 Hz, 2H), 7.22 (s, 1H), 7.00 (d, J=9.0 Hz, 2H), 5.20 (s, 1H), 5.1 (s, 1H), 4.86 (s, 2H), 3.92-3.89 (m, 5H), 3.68 (s, 2H), 3.31 (m, 2H), 1.88 (s, 3H). ¹³C NMR (100 MHz, d₄-MeOH): δ 151.2, 137.9, 137.8, 134.9, 132.7, 120.6, 118.8, 118.1, 114.2, 101.3, 54.1, 47.3, 45.6, 44.8, 34.2, 20.7.

4-[[2-(2-Bromo-allylamino)-ethyl]-(3-methyl-3H-imidazol-4-ylmethyl)-amino]-benzonitrile (7n, X=CN, R¹=2-Bromo-allyl, R³=Methyl): Reaction of 5c according to procedure A, Yield 60%. ¹H NMR (400 MHz, d₄-MeOH): δ 8.92 (s, 1H), 7.57 (d, J=9.0 Hz, 2H), 7.22 (s, 1H), 7.00 (d, J=9.0 Hz, 2H), 4.86 (s, 2H), 4.03 (d, J=2.5 Hz, 2H), 3.92-3.89 (m, 5H), 3.42 (t, J=7.2 Hz, 2H), 3.25 (t, J=2.5 Hz, 1H). ¹³C NMR (100 MHz, d₄-MeOH): δ151.2, 137.9, 135.0, 132.6, 126.5, 120.6, 118.8, 114.3, 114.2, 101.5, 47.3, 45.6, 44.4, 37.7, 34.2.

N-tert-Butyl-2-{2-[(4-cyano-phenyl)-(3-methyl-3H-imidazol-4-ylmethyl)-amino]-ethylamino}-acetamide (7o, X=CN, R¹=N-tert-Butylacetamido, R³=Methyl): Reaction of 5c according to procedure A, Yield 52%. ¹H NMR (400 MHz, d₄-MeOH): δ 8.98 (s, 1H), 7.56 (d, J=9.01 Hz, 2H), 7.28 (s, 1H), 7.01 (d, J=9.01 Hz, 2H), 4.86 (s, 2H), 4.83 (s, 2H), 3.91 (s, 3H), 3.83 (t, J=7.13 Hz, 2H), 3.23 (t, J=7.13 Hz, 2H), 1.32 (s, 9H). ¹³C NMR (100 MHz, d₄-MeOH): δ 165.6, 151.3, 140.2, 135.0, 132.7, 122.7, 120.6, 114.3, 101.3, 52.7, 52.2, 45.6, 37.4, 34.5, 29.1, 28.8.

4-{(3-Methyl-3H-imidazol-4-ylmethyl)-[2-(2-pyrrol-1-yl-ethylamino)-ethyl]-amino}-benzonitrile (7p, X=CN, R¹=Ethylpyrrole, R³=Methyl): Reaction of 5c according to procedure A, Yield 23%. ¹H NMR (400 MHz, d₄-MeOH): δ 8.81 (s, 1H), 7.46 (d, J=8.97 Hz, 2H), 7.07 (s, 1H), 6.85 (d, J=9.13 Hz, 2H), 6.68 (t, J=2.06 Hz, 2H), 6.02 (t, J=2.03 Hz, 2H), 4.69 (s, 2H), 4.21 (t, J=6.20 Hz, 2H), 3.79 (s, 3H), 3.71 (t, J=7.52 Hz, 2H), 3.38 (t, J=6.22 Hz, 2H), 3.03 (t, J=7.50 Hz, 2H). ¹³C NMR (100 MHz, d₄-MeOH): δ 151.6, 138.4, 135.4, 133.0, 122.2, 120.9, 119.3, 114.7, 110.8, 101.9, 50.1, 47.9, 46.9, 46.0, 45.7, 34.6.

4-[[2-(Cyclohexylmethyl-amino)-ethyl]-(3-methyl-3H-imidazol-4-ylmethyl)-amino]-benzonitrile (7q, X=CN, R¹=Cyclohexylmethyl, R³=Methyl): Reaction of 5c according to procedure A, Yield 59%. ¹H NMR (400 MHz, d₄-MeOH): δ 8.84 (s, 1H), 7.48 (d, J=9.09 Hz, 2H), 7.14 (s, 1H), 6.91 (d, J=9.13 Hz, 2H), 4.76 (s, 2H), 3.82 (s, 3H), 3.78 (t, J=7.45 Hz, 2H), 3.19 (obscured), 2.84 (d, J=6.96 Hz, 2H), 1.68 (m, 6H), 1.22 (m, 3H), 0.94 (m, 2H). ¹³C NMR (100 MHz, d₄-MeOH): δ 151.7, 138.4, 135.4, 133.1, 121.0, 119.2, 114.6, 101.8, 55.8, 47.7, 46.0, 37.0, 34.6, 31.8, 27.4, 26.9.

4-((3-Methyl-3H-imidazol-4-ylmethyl)-{2-[(tetrahydro-pyran-4-ylmethyl)-amino]-ethyl}-amino)-benzonitrile (7r, X=CN, R¹=Tetrahydropyran-4-ylmethyl, R³=Methyl): Molecular sieves were added to a solution of tetrahydropyran-4-carbaldehyde (25 mg, 0.22 mmol), 4-[(2-amino-ethyl)-(3-methyl-3H-imidazol-4-ylmethyl)-amino]-benzonitrile di-trifluoroacetic acid salt (0.11 g, 0.22 mmol), and acetic acid (13 μL, 0.23 mmol) in dry MeOH (2.0 mL), and the reaction mixture was stirred at room temperature for 1 hour. NaCNBH₃ (20 mg, 0.33 mmol) was then added in one portion, and stirring continued for a further hour. Brine was added, and the reaction was extracted into EtOAc. The organic layer was purified by flash column chromatography (1:5:50 Et₃N/MeOH/CH₂Cl₂) to provide the title compound in 90% yield. ¹H NMR (400 MHz, d₄-MeOH): δ 7.63 (s, 1H), 7.50 (d, J=9.05 Hz, 2H), 6.97 (d, J=9.05 Hz, 2H), 6.70 (s, 1H), 4.70 (s, 2H), 3.93 (dd, J=10.66, 3.98 Hz, 2H), 3.70-3.64 (m, 5H), 3.40 (dt, J=11.82, 1.64 Hz, 2H), 2.91 (t, J=7.47 Hz, 2H), 2.63 (d, J=6.89 Hz, 2H), 1.82 (m, 1H), 1.67 (dd, J=13.11, 1.89 Hz, 2H), 1.36 (t, J=7.28 Hz, 2H). ¹³C NMR (100 MHz, d₄-MeOH): δ 152.5, 140.3, 134.8, 129.6, 128.1, 121.3, 114.1, 99.6, 56.3, 53.8, 47.9, 46.9, 45.9, 35.6, 32.3, 32.2.

4-[[2-(2-Methyl-benzylamino)-ethyl]-(3-methyl-3H-imidazol-4-ylmethyl)-amino]-benzonitrile (7x, X=CN, R¹=o-Methylbenzyl, R³=Methyl): Reaction of 5c according to procedure A, Yield 62%. ¹H NMR (400 MHz, d₄-MeOH): δ 8.91 (s, 1H), 7.56 (d, J=8.67 Hz, 2H), 7.45 (d, J=7.43 Hz, 2H), 7.34-7.26 (m, 3H), 7.20 (s, 1H), 7.00 (d, J=8.67 Hz, 2H), 4.86 (s, 2H), 4.32 (s, 2H), 3.93-3.89 (m, 5H), 3.43 (t, J=7.53 Hz, 2H), 2.44 (s, 3H). ¹³C NMR (100 MHz, d₄-MeOH): δ 151.3, 138.8, 138.0, 135.0, 132.7, 132.2, 131.3, 130.9, 130.8, 127.8, 120.6, 118.8, 114.3, 101.4, 50.0, 47.4, 45.6, 45.2, 34.2, 19.2.

4-[[2-(3-Methyl-benzylamino)-ethyl]-(3-methyl-3H-imidazol-4-ylmethyl)-amino]-benzonitrile (7y, X=CN, R¹=m-Methylbenzyl, R³=Methyl): Reaction of 5c according to procedure A, Yield 46%. ¹H NMR (400 MHz, d₄-MeOH): δ 8.91 (s, 1H), 7.55 (d, J=8.99 Hz, 2H), 7.35-7.26 (m, 4H), 7.20 (s, 1H), 6.97 (d, J=8.99 Hz, 2H), 4.85 (s, 2H), 4.23 (s, 2H), 3.89-3.86 (m, 5H), 3.35 (t, J=7.40 Hz, 2H), 2.37 (s, 3H). ¹³C NMR (100 MHz, d₄-MeOH): □ 151.2, 140.4, 138.0, 134.9, 132.7, 132.3, 131.6, 131.4, 130.2, 128.0, 120.6, 118.8, 114.3, 101.4, 52.7, 47.5, 45.6, 44.8, 34.2, 21.3.

4-[[2-(4-Methyl-benzylamino)-ethyl]-(3-methyl-3H-imidazol-4-ylmethyl)-amino]-benzonitrile (7z, X=CN, R¹=p-Methylbenzyl, R³=Methyl): Reaction of 5c according to procedure A, Yield 29%. ¹H NMR (400 MHz, d₄-MeOH): δ 8.91 (s, 1H), 7.55 (d, J=8.85 Hz, 2H), 7.38 (d, J=7.84 Hz, 2H), 7.26 (d, J=7.84 Hz, 2H), 7.20 (s, 1H), 6.96 (d, J=8.85 Hz, 2H), 4.84 (s, 2H), 4.22 (s, 2H), 3.89-3.85 (m, 5H), 3.33 (t, J=7.22 Hz, 2H), 2.36 (s, 3H). ¹³C NMR (100 MHz, d₄-MeOH): δ 151.2, 141.1, 138.0, 134.9, 132.6, 131.0, 130.9, 129.3, 120.6, 118.8, 114.2, 101.4, 52.5, 47.5, 45.6, 44.6, 34.2, 21.2.

4-((3-Methyl-3H-imidazol-4-ylmethyl)-{2-[(pyridin-2-ylmethyl)-amino]-ethyl}-amino)-benzonitrile (7aa, X=CN, R¹=2-Pyridyl, R³=Methyl): Reaction of 5c according to procedure A, Yield 57%. ¹H NMR (400 MHz, d₄-MeOH): δ 8.95 (s, 1H), 8.64 (d, J=4.73 Hz, 1H), 7.89 (dt, J=7.74, 1.68 Hz, 1H), 7.59 (d, J=8.99 Hz, 2H), 7.45 (d, J=7.83 Hz, 1H), 7.44 (dd, J=7.36, 5.07 Hz, 1H), 7.26 (s, 1H), 7.03 (d, J=9.04 Hz, 2H), 4.90 (s, 2H), 4.48 (s, 2H), 3.98 (t, J=7.20 Hz, 2H), 3.93 (s, 3H), 3.45 (t, J=7.18 Hz, 2H). ¹³C NMR (100 MHz, d₄-MeOH): δ 152.7, 151.7, 150.9, 139.4, 138.4, 135.4, 133.1, 125.5, 124.6, 121.0, 119.2, 114.8, 101.9, 52.4, 48.8, 47.9, 46.1, 34.7.

4-((3-Methyl-3H-imidazol-4-ylmethyl)-{2-[(pyridin-3-ylmethyl)-amino]-ethyl}-amino)-benzonitrile (7ab, X=CN, R¹=3-Pyridyl, R³=Methyl): Reaction of 5c according to procedure A, Yield 64%. ¹H NMR (400 MHz, d₄-MeOH): δ 8.92 (br s, 2H), 8.82 (d, J=5.36 Hz, 1H), 8.47 (d, J=6.41 Hz, 1H), 7.90 (dd, J=7.92, 5.45 Hz, 1H), 7.57 (d, J=9.00 Hz, 2H), 7.23 (s, 1H), 7.02 (d, J=9.02 Hz, 2H), 4.89 (s, 2H), 4.48 (s, 2H), 3.95 (t, J=7.17 Hz, 2H), 3.92 (s, 3H), 3.49 (t, J=7.12 Hz, 2H). ¹³C NMR (100 MHz, d₄-MeOH): δ 151.7, 148.3, 147.7, 145.6, 138.3, 135.4, 133.1, 132.0, 127.7, 121.0, 119.1, 114.7, 101.8, 49.7, 47.9, 46.0, 45.9, 34.6.

4-((3-Methyl-3H-imidazol-4-ylmethyl)-{2-[(pyridin-4-ylmethyl)-amino]-ethyl}-amino)-benzonitrile (7ac, X=CN, R¹=4-Pyridyl, R³=Methyl): Reaction of 5c according to procedure A, Yield 60%. ¹H NMR (400 MHz, d₄-MeOH): δ 8.94 (s, 1H), 8.83 (d, J=6.14 Hz, 2H), 7.96 (d, J=6.33 Hz, 2H), 7.58 (d, J=8.93 Hz, 2H), 7.24 (s, 1H), 7.03 (d, J=9.00 Hz, 2H), 4.88 (s, 2H), 4.54 (s, 2H), 3.97 (t, J=7.13 Hz, 2H), 3.92 (s, 3H), 3.50 (t, J=7.39 Hz, 2H). ¹³C NMR (100 MHz, d₄-MeOH): δ 151.6, 148.9, 147.4, 138.4, 135.4, 133.1, 128.0, 121.0, 119.2, 114.7, 101.8, 51.4, 47.9, 46.2, 46.0, 34.6.

4-[[2-(3-Cyano-benzylamino)-ethyl]-(3-methyl-3H-imidazol-4-ylmethyl)-amino]-benzonitrile (7ad, X=CN, R¹=m-Cyanobenzyl, R³=Methyl): Reaction of 5c according to procedure A, Yield 49%. ¹H NMR (400 MHz, d₄-MeOH): δ 8.91 (s, 1H), 7.90 (s, 1H), 7.85-7.81 (m, 2H), 7.64 (t, J=7.80 Hz, 1H), 7.56 (d, J=8.77 Hz, 2H), 7.21 (s, 1H), 6.99 (d, J=8.77 Hz, 2H), 4.86 (s, 2H), 4.35 (s, 2H), 3.93-3.90 (m, 5H), 3.41 (t, J=7.27 Hz, 2H). ¹³C NMR (100 MHz, d₄-MeOH): δ 151.6, 138.4, 136.3, 135.4, 135.2, 134.8, 134.6, 133.1, 131.8, 121.0, 119.4, 119.2, 114.7, 114.6, 114.3, 101.8, 52.1, 47.9, 46.1, 45.6, 34.6.

4-[[2-(4-Cyano-benzylamino)-ethyl]-(3-methyl-3H-imidazol-4-ylmethyl)-amino]-benzonitrile (7ae, X=CN, R¹=p-Cyanobenzyl, R³=Methyl): Reaction of 5c according to procedure A, Yield 35%. ¹H NMR (400 MHz, d₄-MeOH): δ 8.91 (s, 1H), 7.82 (d, J=8.19 Hz, 2H), 7.70 (d, J=8.19 Hz, 2H), 7.56 (d, J=8.88 Hz, 2H), 7.21 (s, 2H), 6.99 (d, J=8.88 Hz, 2H), 4.86 (s, 2H), 4.37 (s, 2H), 3.92-3.90 (m, 5H), 3.41 (t, J=7.33 Hz, 2H). ¹³C NMR (100 MHz, d₄-MeOH): δ 151.2, 138.0, 137.7, 135.0, 134.0, 132.6, 132.0, 120.6, 119.0, 118.8, 114.6, 114.2, 101.4, 52.0, 47.4, 45.6, 45.3, 34.2.

4-[{2-[(Biphenyl-3-ylmethyl)-amino]-ethyl}-(3-methyl-3H-imidazol-4-ylmethyl)-amino]-benzonitrile (7af, X=CN, R¹=3-Phenylbenzyl, R³=Methyl): Reaction of 5c according to procedure A, Yield 58%. ¹H NMR (400 MHz, d₄-MeOH): δ 8.82 (s, 1H), 7.72 (s, 1H), 7.63 (d, J=7.60 Hz, 1H), 7.56 (d, J=7.19 Hz, 2H), 7.45 (m, 6H), 7.29 (t, J=7.24 Hz, 1H), 7.11 (s, 1H), 6.89 (d, J=9.04 Hz, 2H), 4.76 (s, 2H), 4.23 (s, 2H), 3.82 (t, J=7.33 Hz, 2H), 3.80 (s, 3H), 3.32 (t, J=7.23 Hz, 2H). ¹³C NMR (100 MHz, d₄-MeOH): δ 151.6, 143.9, 141.8, 138.4, 135.4, 133.5, 133.1, 131.3, 130.4, 130.3, 130.1, 129.6, 129.3, 128.4, 121.0, 119.2, 114.7, 101.7, 53.1, 47.9, 46.0, 45.2, 34.6.

4-[{2-[(Biphenyl-4-ylmethyl)-amino]-ethyl}-(3-methyl-3H-imidazol-4-ylmethyl)-amino]-benzonitrile (7ag, X=CN, R¹=4-Phenylbenzyl, R³=Methyl): Reaction of 5c according to procedure A, Yield 64%. ¹H NMR (400 MHz, d₄-MeOH): δ 8.91 (s, 1H), 7.73 (d, J=8.26 Hz, 2H), 7.59 (m, 6H), 7.47 (t, J=7.3 Hz, 2H), 7.40 (t, J=7.36 Hz, 1H), 7.22 (s, 1H), 6.99 (d, J=9.06 Hz, 2H), 4.87 (s, 2H), 4.34 (s, 2H), 3.91 (m, 5H), 3.40 (t, J=7.21 Hz, 2H). ¹³C NMR (100 MHz, d₄-MeOH): δ 151.6, 144.3, 141.7, 138.4, 135.4, 133.0, 132.0, 131.7, 130.4, 129.4, 129.2, 128.4, 121.0, 119.3, 114.7, 101.9, 52.8, 47.9, 46.0, 45.2, 34.6.

4-{(3-Methyl-3H-imidazol-4-ylmethyl)-[2-(3-pyrrol-1-yl-benzylamino)-ethyl]-amino}-benzonitrile (7ah, X=CN, R¹=3-Pyrrolebenzyl, R³=Methyl): Reaction of 5c according to procedure A, Yield 62%. ¹H NMR (400 MHz, d₄-MeOH): δ 8.82 (s, 1H), 7.59 (s, 1H), 7.46 (m, 4H), 7.28 (d, J=7.43 Hz, 1H), 7.15 (t, J=2.26 Hz, 2H), 7.12 (s, 1H), 6.89 (d, J=9.07 Hz, 2H), 6.23 (t, J=2.23 Hz, 2H), 4.76 (s, 2H), 4.25 (s, 2H), 3.81 (obscured), 3.80 (s, 3H), 3.31 (t, J=7.56 Hz, 2H). ¹³C NMR (100 MHz, d₄-MeOH): δ 151.6, 143.1, 138.4, 135.4, 134.5, 133.1, 132.1, 128.1, 122.5, 122.2, 120.9, 120.3, 119.2, 114.7, 112.4, 101.9, 52.8, 47.9, 46.1, 45.3, 34.6.

4-{(3-Methyl-3H-imidazol-4-ylmethyl)-[2-(4-pyrrol-1-yl-benzylamino)-ethyl]-amino}-benzonitrile (7ai, X=CN, R¹=4-Pyrrolebenzyl, R³=Methyl): Reaction of 5c according to procedure A, Yield 58%. ¹H NMR (400 MHz, d₄-MeOH): δ 8.81 (s, 1H), 7.48 (m, 5H), 7.45 (s, 1H), 7.14 (t, J=2.16 Hz, 2H), 6.88 (d, J=9.10 Hz, 2H), 6.21 (t, J=2.17 Hz, 2H), 4.75 (s, 2H), 4.20 (s, 2H), 3.80 (m, 5H), 3.28 (t, J=7.20 Hz, 2H). ¹³C NMR (100 MHz, d₄-MeOH): δ 151.6, 138.4, 135.4, 133.1, 129.4, 121.5, 121.0, 120.3, 119.2, 114.7, 112.5, 111.4, 101.8, 52.5, 47.9, 46.0, 45.2, 34.6.

1-Methyl-1H-imidazole-4-sulfonic acid allyl-{2-[(4-cyano-phenyl)-(3-methyl-3H-imidazol-4-ylmethyl)-amino]-ethyl}-amide (8l, X=CN, R¹=Allyl, R²=4-methyl-1H-imidazole, R³=Methyl): Reaction of 7l according to procedure C, Yield 68%. ¹H NMR (400 MHz, d₄-MeOH): δ 8.81 (s, 1H), 7.67 (s, 1H), 7.63 (s, 1H), 7.45 (d, J=9.15 Hz, 2H), 7.21 (s, 1H), 6.86 (d, J=9.18 Hz, 2H), 5.60 (m, 1H), 5.05 (m, 2H), 4.73 (s, 2H), 3.80 (s, 3H), 3.68 (m, 5H), 3.57 (m, 2H), 3.28 (m, 2H). ¹³C NMR (100 MHz, d₄-MeOH): δ 152.2, 141.9, 139.7, 138.2, 135.2, 133.2, 133.3, 127.0, 122.4, 121.2, 119.7, 114.4, 100.8, 54.0, 31.4, 46.0, 41.8, 34.7, 34.6. HRMS calcd for C₂₁H₂₆N₇O₂S⁺: 440.1869. found 440.1855. Retention time for analytical rpHPLC: condition (I) 12.31, (II) 17.86 minutes.

1-Methyl-1H-imidazole-4-sulfonic acid {2-[(4-cyano-phenyl)-(3-methyl-3H-imidazol-4-ylmethyl)-amino]-ethyl}-(2-methyl-allyl)-amide, (8m, X=CN, R¹=2-Methyl-allyl, R²=1-Methyl-1H-imidazole, R³=Methyl): Reaction of 7m according to procedure C, Yield 71%. ¹H NMR (400 MHz, d₄-MeOH): δ 8.91 (s, 1H), 7.78 (s, 1H), 7.74 (s, 1H), 7.53 (d, J=9.03 Hz, 2H), 7.29 (s, 1H), 6.95 (d, J=9.03 Hz, 2H), 4.79 (s, 2H), 3.89 (s, 3H), 3.78 (s, 3H), 3.73-3.69 (m, 4H), 3.36 (t, J=3.95 Hz, 2H), 1.69 (s, 3H). ¹³C NMR (100 MHz, d₄-MeOH): □ 151.7, 142.6, 141.4, 139.2, 137.9, 134.8, 132.8, 126.6, 120.8, 119.3, 114.0, 100.4, 57.3, 50.8, 46.0, 45.2, 34.3, 34.3, 34.2, 20.0. HRMS (ESI): m/z calcd for C₂₂H₂₇N₇O₂SH⁺ 454.2025. found 454.2013. Retention time for analytical rpHPLC: condition (I) 14.29, (II) 18.51 minutes.

1-Methyl-1H-imidazole-4-sulfonic acid (2-bromo-allyl)-{2-[(4-cyano-phenyl)-(3-methyl-3H-imidazol-4-ylmethyl)-amino]-ethyl}-amide (8n, X=CN, R¹=2-Bromo-allyl, R²=1-Methyl-1H-imidazole, R³=Methyl): Reaction of 7n according to procedure C, Yield 67%. ¹H NMR (400 MHz, d₄-MeOH): [symbol] 8.90 (s, 1H), 7.76 (s, 1H), 7.74 (s, 1H), 7.55 (d, J=9.03 Hz, 2H), 7.32 (s, 1H), 7.00 (d, J=9.03 Hz, 2H), 4.85 (s, 2H), 4.07 (d, J=2.42 Hz, 2H), 3.91 (s, 3H), 3.82 (t, J=6.75 Hz, 2H), 3.77 (s, 3H), 3.55 (t, J=6.75 Hz, 2H), 2.64 (t, J=2.42 Hz, 1H). ¹³C NMR (100 MHz, d₄-MeOH): δ 151.9, 141.3, 138.6, 137.8, 134.8, 133.0, 127.1, 120.8, 119.3, 114.1, 100.4, 78.2, 75.3, 50.5, 46.0, 45.6, 39.3, 34.3, 34.3 HRMS (ESI): m/z calcd for C₂₁H₂₄BrN₇O₂SH⁺ 518.0974. found 518.0980. Retention time for analytical rpHPLC: condition (I) 10.70, (II) 16.36 minutes.

N-tert-Butyl-2-[{2-[(4-cyano-phenyl)-(3-methyl-3H-imidazol-4-ylmethyl)-amino]-ethyl}-(1-methyl-1H-imidazole-4-sulfonyl)-amino]-acetamide (8o, X=CN, R¹=N-tert-Butylacetamido, R²=1-Methyl-1H-imidazole, R³=Methyl): Reaction of 7o according to procedure C, Yield 83%. ¹H NMR (400 MHz, d₄-MeOH): δ 8.94 (s, 1H), 7.77 (s, 1H), 7.66 (s, 1H), 7.52 (d, J=8.95 Hz, 2H), 7.29 (s, 1H), 6.92 (d, J=8.95 Hz, 2H), 4.82 (s, 4H), 3.91 (s, 3H), 3.74 (s, 3H), 3.65 (t, J=6.23 Hz, 2H), 3.21 (t, J=6.23 Hz, 2H), 1.32 (s, 9H). ¹³C NMR (100 MHz, d₄-MeOH): δ 165.6, 151.7, 141.1, 140.5, 140.0, 134.8, 133.0, 125.8, 123.1, 120.8, 113.9, 100.3, 52.7, 52.2, 51.5, 46.0, 41.3, 34.5, 34.3, 28.7. HRMS calcd for C₂₄H₃₂N₈O₃SH⁺ 513.2396. found 513.2392. Retention time for analytical rpHPLC: condition (I) 12.14, (II) 17.82 minutes.

1-Methyl-1H-imidazole-4-sulfonic acid {2-[(4-cyano-phenyl)-(3-methyl-3H-imidazol-4-ylmethyl)-amino]-ethyl}-(2-pyrrol-1-yl-ethyl)-amide (8p, X=CN, R¹=2-Pyrrol-1-yl-ethyl, R²=4-methyl-1H-imidazole, R³=Methyl): Reaction of 7p according to procedure C, Yield 71%. ¹H NMR (400 MHz, d₄-MeOH): δ 8.79 (s, 1H), 7.69 (s, 1H), 7.67 (s, 1H), 7.42 (d, J=9.09 Hz, 2H), 7.01 (s, 1H), 6.78 (d, J=9.11 Hz, 2H), 6.59 (d, J=1.95, 3.57 Hz, 2H), 5.89 (d, J=2.12 Hz, 2H), 4.90 (s, 2H), 4.02 (t, J=5.37 Hz, 2H), 3.78 (s, 3H), 3.69 (s, 3H), 3.45 (t, J=5.63 Hz, 2H), 3.12 (m, 2H), 3.02 (m, 2H). ¹³C NMR (100 MHz, d₄-MeOH): δ 150.9, 140.9, 138.3, 137.1, 134.1, 132.2, 126.1, 121.4, 120.1, 118.5, 113.1, 108.6, 99.6, 52.6, 50.3, 50.2, 49.9, 44.6, 33.6, 33.5. HRMS calcd for C₂₄H₂₉N₈O₂S⁺ 493.2129. found 493.2122. Retention time for analytical rpHPLC: condition (I) 11.26, (II) 14.39 minutes.

1-Methyl-1H-imidazole-4-sulfonic acid {2-[(4-cyano-phenyl)-(3-methyl-3H-imidazol-4-ylmethyl)-amino]-ethyl}-cyclohexylmethyl-amide (8q, X=CN, R¹=Cyclohexylmethyl, R²=4-Methyl-1H-imidazole, R³=Methyl): Reaction of 7q according to procedure C, Yield 75%. ¹H NMR (400 MHz, d₄-MeOH): δ 8.82 (s, 1H), 7.67 (s, 1H), 7.65 (s, 1H), 7.47 (d, J=9.04 Hz, 2H), 7.21 (s, 1H), 6.91 (d, J=9.14 Hz, 2H), 4.78 (s, 2H), 3.82 (s, 3H), 3.69 (m, 5H), 3.30 (m, 2H), 2.82 (d, J=7.32 Hz, 2H), 1.57 (m, 5H), 1.34 (m, 1H), 1.05 (m, 3H), 0.76 (m, 2H). ¹³C NMR (100 MHz, d₄-MeOH): δ 152.2, 141.7, 139.5, 138.3, 135.3, 133.4, 127.0, 121.2, 119-61 114.3, 100.8, 58.6, 51.61 48.4, 45.8, 38.4, 34.7, 32.3, 27.9, 27.3. HRMS calcd for C₂₅H₃₄N₇O₂S⁺: 496.2495. found 496.2497. Retention time for analytical rpHPLC: condition (I) 14.92, (II) 27.55 minutes.

1-Methyl-1H-imidazole-4-sulfonic acid {2-[(4-cyano-phenyl)-(3-methyl-3H-imidazol-4-ylmethyl)-amino]-ethyl}-(tetrahydro-pyran-4-ylmethyl)-amide (8r, X=CN, R¹=Tetrahydropyran-4-ylmethyl, R²=1-Methyl-1H-imidazole, R³=Methyl): Reaction of 7r according to procedure C, Yield 67%. ¹H NMR (400 MHz, d₄-MeOH): δ 8.91 (s, 1H), 7.77 (s, 1H), 7.76 (s, 1H), 7.56 (d, J=8.95 Hz, 2H), 7.27 (s, 1H), 7.01 (d, J=8.95 Hz, 2H), 4.87 (s, 2H), 3.91 (s, 3H), 3.89-3.80 (m, 4H), 3.78 (s, 3H), 3.41 (t, J=7.11 Hz, 2H), 3.26 (t, J=10.99 Hz, 2H), 2.99 (d, J=7.30 Hz, 2H), 1.73 (m, 1H), 1.58 (d, J=13.02 Hz, 2H), 1.19 (m, 2H). ¹³C NMR (100 MHz, d₄-MeOH): □ 151.7, 141.4, 138.9, 137.9, 134.9, 132.9, 126.8, 120.8, 119.1, 114.0, 100.4, 68.5, 57.5, 51.1, 48.1, 45.4. HRMS (ESI): m/z calcd for C₂₄H₃₁N₇O₃SH⁺ 498.2287. found 498.2287. Retention time for analytical rpHPLC: condition (I) 10.97, (II) 19.75 minutes.

1-Methyl-1H-imidazole-4-sulfonic acid {2-[(4-cyano-phenyl)-(3-methyl-3H-imidazol-4-ylmethyl)-amino]-ethyl}-piperidin-4-ylmethyl-amide (8s, X=CN, R¹=Piperidin-4-ylmethyl, R²=1-Methyl-1H-imidazole, R³=Methyl): 4-{[{2-[(4-Cyano-phenyl)-(3-methyl-3H-imidazol-4-ylmethyl)-amino]-ethyl}-(1-methyl-1H-imidazole-4-sulfonyl)-amino]-methyl}-piperidine-1-carboxylic acid tert-butyl ester (0.15 g, 0.25 mmol) was dissolved in TFA (2.0 mL) and the solution stirred at room temperature for 15 minutes. The solvent was removed under reduced pressure, and the crude product purified by rpHPLC to provide the title compound. Yield 94%. ¹H NMR (400 MHz, d₄-MeOH): δ 8.91 (s, 1H), 7.78 (s, 1H), 7.77 (s, 1H), 7.55 (d, J=9.02 Hz, 2H), 7.23 (s, 1H), 7.02 (d, J=9.02 Hz, 2H), 4.87 (s, 2H), 3.91 (s, 3H), 3.82 (t, J=7.51 Hz, 2H), 3.78 (s, 3H), 3.42-3.34 (m, 4H), 3.08 (d, J=7.12 Hz, 2H), 2.86 (dt, J=2.31, 11.46 Hz, 2H), 1.93-1.84 (m, 3H), 1.39 (m, 2H). ¹³C NMR (100 MHz, d₄-MeOH): [symbol] 151.7, 141.5, 138.6, 137.8, 134.9, 132.9, 126.9, 120.8, 118.9, 114.0, 100.3, 56.5, 51.2, 51.1, 48.2, 45.3, 44.7, 34.4, 34.3, 27.6. HRMS (ESI): m/z calcd for C₂₄H₃₂N₈O₂SH⁺ 497.2447. found 497.2444. Retention time for analytical rpHPLC: condition (I) 11.33, (II) 18.12 minutes.

1-Methyl-1H-imidazole-4-sulfonic acid (1-acetyl-piperidin-4-ylmethyl)-{2-[(4-cyano-phenyl)-(3-methyl-3H-imidazol-4-ylmethyl)-amino]-ethyl}-amide (8t, X=CN, R¹=1-Acetyl-piperidin-4-ylmethyl, R²=1-Methyl-1H-imidazole, R³=Methyl): To a solution of 1-methyl-1H-imidazole-4-sulfonic acid {2-[(4-cyano-phenyl)-(3-methyl-3H-imidazol-4-ylmethyl)-amino]-ethyl}-piperidin-4-ylmethyl-amide CF₃CO₂H salt (8s) (37 mg, 0.061 mmol) and TEA (51 μL, 0.37 mmol) in DMF (0.30 mL) at 0° C., was added acetic anhydride (7.0 □L, 0.070 mmol). The reaction was stirred at 0° C. for 10 min, then diluted with acetonitrile and purified directly by rpHPLC to provide the title compound. Yield 85%. ¹H NMR (400 MHz, d₄-MeOH): δ 8.91 (s, 1H), 7.77 (s, 1H), 7.75 (s, 1H), 7.56 (d, J=9.06 Hz, 2H), 7.27 (s, 1H), 7.01 (d, J=9.06 Hz, 2H), 4.87 (s, 2H), 4.41 (d, J=13.27 Hz, 1H), 3.91 (s, 3H), 3.88-3.81 (m, 3H), 3.78 (s, 3H), 3.42 (t, J=7.18 Hz, 2H), 3.02-2.94 (m, 3H), 2.51 (dt, J=2.26, 12.64 Hz, 1H), 2.07 (s, 3H), 1.79-1.67 (m, 3H), 1.06 (m, 2H). ¹³C NMR (100 MHz, d₄-MeOH): d 171.4, 151.8, 141.4, 138.9, 137.9, 134.9, 133.0, 126.8, 120.8, 119.1, 114.0, 100.4, 57.1, 51.2, 45.4, 42.5, HRMS (ESI): m/z calcd for C₂₆H₃₄N₈O₃SH⁺ 539.2553. found 539.2544. Retention time for analytical rpHPLC: condition (I) 12.42, (II) 21.08 minutes.

1-Methyl-1H-imidazole-4-sulfonic acid {2-[(4-cyano-phenyl)-(3-methyl-3H-imidazol-4-ylmethyl)-amino]-ethyl}-(1-isobutyryl-piperidin-4-ylmethyl)-amide (8u, X=CN, R¹=1-Isobutyryl-piperidin-4-ylmethyl, R²=1-Methyl-1H-imidazole, R³=Methyl): Acylation with isobutyric anhydride following the same procedure as described above provided the title compound in yield 73%. ¹H NMR (400 MHz, d₄-MeOH): δ 8.91 (s, 1H), 7.76 (s, 1H), 7.75 (s, 1H), 7.57 (d, J=8.99 Hz, 2H), 7.29 (s, 1H), 7.01 (d, J=8.99 Hz, 2H), 4.86 (s, 2H), 4.45 (d, J=12.09 Hz, 1H), 4.00 (d, J=13.70 Hz, 1H), 3.91 (s, 3H), 3.83-3.78 (m, 5H), 3.44 (m, 2H), 3.01-2.89 (m, 4H), 2.50 (t, J=11.62 Hz, 1H), 1.78-1.68 (m, 3H), 1.14-0.95 (m, 8H). ¹³C NMR (100 MHz, d₄-MeOH): δ 177.7, 151.8, 141.4, 138.8, 137.9, 134.9, 132.9, 126.8, 120.8, 119.1, 114.0, 110.4, 57.1, 51.2, 46.5, 45.4, 42.8, 36.7, 34.3, 31.8, 31.1, 30.7, 19.9, 19.7. ^{˜}HRMS (ESI): m/z calcd for C₂₈H₃₈N₈O₃S₁+567.2866. found 567.2840. Retention time for analytical rpHPLC: condition (I) 12.76, (II) 22.13 minutes.

4-{[{2-[(4-Cyano-phenyl)-(3-methyl-3H-imidazol-4-ylmethyl)-amino]-ethyl}-(1-methyl-1H-imidazole-4-sulfonyl)-amino]-methyl}-piperidine-1-carboxylic acid tert-butyl ester (8v, X=CN, R¹=Methylpiperidine-1-carboxylic acid tert-butyl ester, R²=1-Methyl-1H-imidazole, R³=Methyl): Reaction of 6c according to procedure B, Yield 47%. ¹H NMR (400 MHz, d₄-MeOH): δ 8.90 (s, 1H), 7.76 (s, 1H), 7.75 (s, 1H), 7.58 (d, J=9.07 Hz, 2H), 7.29 (s, 1H), 7.01 (d, J=9.07 Hz, 2H), 4.87 (s, 2H), 3.99 (d, J=13.22 Hz, 2H), 3.82-3.78 (m, 5H), 3.42 (t, J=6.99 Hz, 2H), 2.97 (d, J=6.85 Hz, 2H), 2.61 (br s, 2H), 1.65-1.62 (m, 3H), 1.44 (s, 9H), 1.01 (m, 2H). ¹³C NMR (100 MHz, d₄-MeOH): δ 156.5, 151.8, 141.4, 138.9, 137.8, 134.9, 133.0, 126.8, 120.8, 119.1, 114.0, 100.4, 81.0, 57.3, 51.2 48.2, 45.4, 36.5, 34.3, 34.2, 30.8, 28.7. HRMS (ESI): m/z calcd for C₂₉H₄₀N₈O₄SH⁺ 597.2971. found 597.2974. Retention time for analytical rpHPLC: condition (I) 13.25, (II) 24.68 minutes.

1-Methyl-1H-imidazole-4-sulfonic acid {2-[(4-cyano-phenyl)-(3-methyl-3H-imidazol-4-ylmethyl)-amino]-ethyl}-(1-pyrimidin-2-yl-piperidin-4-ylmethyl)-amide (8w, X=CN, R¹=1-Pyrimidin-2-yl-piperidin-4-ylmethyl, R²=1-Methyl-1H-imidazole, R³=Methyl): To the solution of 1-methyl-1H-imidazole-4-sulfonic acid {2-[(4-cyano-phenyl)-(3-methyl-3H-imidazol-4-ylmethyl)-amino]-ethyl}-piperidin-4-ylmethyl-amide CF₃CO₂H salt (8s) (35 mg, 0.057 mmol) in THF (600 μL) at 0° C. was added LDA (2.0 M, 71 □L, 0.14 mmol) and the resulting solution stirred for 1 hour. 2-Chloropyrimidine (6.5 mg, 0.057 mmol) was then added, and the reaction was stirred overnight. The reaction was diluted with EtOAc, washed with brine. The organic phase was dared over magnesium sulfate, and solvent was removed under reduced pressure. The residue purified by rpHPLC. Yield 46%. ¹H NMR (400 MHz, d₄-MeOH): δ 8.91 (s, 1H), 8.36 (d, J=4.89 Hz, 2H), 7.77 (s, 1H), 7.75 (s, 1H), 7.57 (d, J=8.90 Hz, 2H), 7.28 (s, 1H), 7.02 (d, J=8.97 Hz, 2H), 6.66 (t, J=4.92 Hz, 1H), 4.88 (s, 2H), 4.59 (d, J=13.33 Hz, 2H), 3.92 (s, 3H), 3.83 (t, J=7.05 Hz, 2H), 3.78 (s, 3H), 3.44 (t, J=7.05 Hz, 2H), 3.01 (d, J=7.16 Hz, 2H), 2.88 (t, J=11.76 Hz, 2H), 1.85-1.80 (m, 3H), 1.14 (m, 2H). ¹³C NMR (100 MHz, d₄-MeOH): δ 160.3, 158.7, 151.8, 141.4, 138.9, 137.9, 134.9, 133.0, 126.8, 120.8, 119.1, 114.0, 110.6, 100.4, 57.3, 51.2, 48.3, 45.4, 45.2, 36.6, 34.4, 34.3, 30.5. HRMS (ESI): m/z calcd for C₂₈H₃₄N₁₀O₂SH⁺ 575.2665. found 575.2661. Retention time for analytical rpHPLC: condition (I) 12.94, (II) 19.66 minutes.

1-Methyl-1H-imidazole-4-sulfonic acid {2-[(4-cyano-phenyl)-(3-methyl-3H-imidazol-4-ylmethyl)-amino]-ethyl}-(2-methyl-benzyl)-amide (8x, X=CN, R¹=o-Methylbenzyl, R²=4-Methyl-1H-imidazole, R³=Methyl): Reaction of 7x according to procedure C, Yield 83%. ¹H NMR (400 MHz, d₄-MeOH): δ 8.87 (s, 1H), 7.84 (s, 1H), 7.81 (s, 1H), 7.38 (d, J=8.91 Hz, 2H), 7.30-7.17 (m, 4H), 7.10 (s, 1H), 6.48 (d, J=8.91 Hz, 2H), 4.40 (s, 2H), 4.25 (s, 2H), 3.82 (s, 3H), 3.81 (s, 3H), 3.29 (br s, 4H), 2.34 (s, 3H). ¹³C NMR (100 MHz, d₄-MeOH): δ 151.5, 141.6, 139.8, 138.2, 137.8, 134.9, 134.7, 132.6, 132.2, 132.1, 129.8, 127.2, 127.0, 120.8, 119.0, 113.5, 100.0, 54.0, 51.0, 45.6, 44.6, 34.4, 34.2, 19.3. HRMS (ESI): m/z calcd for C₂₆H₂₉N₇O₂SH⁺ 504.2182. found 504.2177. Retention time for analytical rpHPLC: condition (I) 14.68, (II) 22.48 minutes.

1-Methyl-1H-imidazole-4-sulfonic acid {2-[(4-cyano-phenyl)-(3-methyl-3H-imidazol-4-ylmethyl)-amino]-ethyl}-(3-methyl-benzyl)-amidem-toluene (8y, X=CN, R¹=m-Methylbenzyl, R²=1-Methyl-1H-imidazole, R³=Methyl): Reaction of 7y according to procedure C, Yield 87%. ¹H NMR (400 MHz, d₄-MeOH): δ 8.88 (s, 1H), 7.82 (s, 1H), 7.78 (s, 1H), 7.39 (d, J=9.07 Hz, 2H), 7.22 (t, J=7.71 Hz, 1H), 7.14-7.11 (m, 4H), 6.56 (d, J=9.07 Hz, 2H), 4.52 (s, 2H), 4.21 (s, 2H), 3.82 (s, 3H), 3.80 (s, 3H), 3.47 (t, J=6.86 Hz, 2H), 3.35 (t, J=6.86 Hz, 2H), 2.28 (s, 3H). ¹³C NMR (100 MHz, d₄-MeOH): δ 151.9, 141.9, 140.2, 139.3, 138.2, 138.0, 135.1, 133.1, 131.4, 130.4, 130.1, 127.9, 127.2, 121.3, 119.5, 114.0, 100.5, 55.7, 51.5, 46.8, 45.4, 34.8, 34.6, 21.9. HRMS (ESI): m/z calcd for C₂₆H₂₉N₇O₂SH⁺ 504.2182. found 504.2186. Retention time for analytical rpHPLC: condition (I) 14.78, (II) 20.99 minutes.

1-Methyl-1H-imidazole-4-sulfonic acid {2-[(4-cyano-phenyl)-(3-methyl-3H-imidazol-4-ylmethyl)-amino]-ethyl}-(4-methyl-benzyl)-amide (8z, X=CN, R¹=p-Methylbenzyl, R²=1-Methyl-1H-imidazole, R³=Methyl): Reaction of 7z according to procedure C, Yield 74%. ¹H NMR (400 MHz, d₄-MeOH): δ 8.88 (d, J=0.82 Hz, 1H), 7.81 (d, J=1.02 Hz, 1H), 7.78 (d, J=1.22 Hz, 1H), 7.38 (d, J=9.04 Hz, 2H), 7.20 (d, J=8.02 Hz, 2H), 7.15 (d, J=1.29 Hz, 1H), 7.12 (d, J=7.87 Hz, 2H), 6.54 (d, J=9.07 Hz, 2H), 4.56 (s, 2H), 4.19 (s, 2H), 3.83 (s, 3H), 3.80 (s, 3H), 3.43 (t, J=7.45 Hz, 2H), 3.34 (t, J=7.45 Hz, 2H), 2.33 (s, 3H). ¹³C NMR (100 MHz, d₄-MeOH): δ 151.5, 141.5, 139.3, 138.8, 137.8, 134.6, 132.8, 130.4, 126.8, 120.8, 119.1, 113.5, 100.0, 55.1, 51.0, 46.4, 45.1, 34.3, 34.2, 21.2. HRMS (ESI): m/z calcd for C₂₆H₂₉N₇O₂SH⁺ 504.2182. found 504.2184. Retention time for analytical rpHPLC: condition (I) 14.92, (II) 21.40 minutes.

1-Methyl-1H-imidazole-4-sulfonic acid {2-[(4-cyano-phenyl)-(3-methyl-3H-imidazol-4-ylmethyl)-amino]-ethyl}-pyridin-2-ylmethyl-amide amide (8aa, X=CN, R¹=2-Pyridylmethyl, R²=4-methyl-1H-imidazole, R³=Methyl): Reaction of 7aa according to procedure C, Yield 62%. ¹H NMR (400 MHz, d₄-MeOH): δ 8.91 (s, 1H), 8.53 (d, J=5.17 Hz, 1H), 7.97 (td, J=1.52, 7.77 Hz, 1H), 7.83 (s, 1H), 7.81 (s, 1H), 7.64 (d, J=7.91 Hz, 1H), 7.48 (d, J=9.10 Hz, 2H), 7.47 (obscured, 1H), 7.21 (s, 1H), 6.80 (d, J=9.11 Hz, 2H), 4.73 (s, 2H), 4.55 (s, 2H), 3.89 (s, 3H), 3.81 (s, 3H), 3.66 (m, 2H), 3.54 (m, 2H). ¹³C NMR (100 MHz, d₄-MeOH): δ 155.9, 150.6, 147.1, 140.6, 140.1, 137.6, 136.8, 133.8, 131.8, 126.1, 124.9, 124.3, 119.8, 118.0, 112.8, 99.3, 54.1, 49.5, 46.6, 44.2, 33.4, 33.2. HRMS calcd for C₂₄H₂₇N₈O₂S⁺: 491.1978. found 491.1970. Retention time for analytical rpHPLC: condition (I) 11.13, (II) 14.43 minutes.

1-Methyl-1H-imidazole-4-sulfonic acid {2-[(4-cyano-phenyl)-(3-methyl-3H-imidazol-4-ylmethyl)-amino]-ethyl}-pyridin-3-ylmethyl-amide (8ab, X=CN, R¹=3-Pyridylmethyl, R²=4-methyl-1H-imidazole, R³=Methyl): Reaction of 7ab according to procedure C, Yield 63%. ¹H NMR (400 MHz, d₄-MeOH): δ 8.90 (s, 1H), 8.62 (s, 1H), 8.56 (d, J=6.49 Hz, 1H), 8.15 (d, J=8.01 Hz, 1H), 7.84 (s, 2H), 7.62 (dd, J=5.26, 7.87 Hz, 1H), 7.47 (d, J=9.05 Hz, 2H), 7.17 (s, 1H), 6.74 (d, J=9.11 Hz, 2H), 4.69 (s, 2H), 4.48 (s, 2H), 3.88 (s, 3H), 3.82 (s, 3H), 3.66 (m, 2H), 3.50 (m, 2H). ¹³C NMR (100 MHz, d₄-MeOH): δ 151.8, 147.4, 146.9, 142.9, 142.2, 138.9, 138.3, 137.3, 135.2, 133.0, 127.7, 127.0, 121.1, 119.4, 114.1, 100.8, 52.6, 51.1, 48.1, 45.6, 34.8, 34.6. HRMS calcd for C₂₄H₂₇N₈O₂S⁺: 491.1978. found 491.1969. Retention time for analytical rpHPLC: condition (I) 11.21, (II) 14.84 minutes.

1-Methyl-1H-imidazole-4-sulfonic acid {2-[(4-cyano-phenyl)-(3-methyl-3H-imidazol-4-ylmethyl)-amino]-ethyl}-pyridin-4-ylmethyl-amide (8ac, X=CN, R¹=4-Pyridylmethyl, R²=4-methyl-1H-imidazole, R³=Methyl): Reaction of 7ac according to procedure C, Yield 71%. ¹H NMR (400 MHz, d₄-MeOH): δ 8.79 (s, 1H), 8.43 (d, J=6.11 Hz, 1H), 7.73 (d, J=6.58 Hz, 1H), 7.49 (s, 1H), 7.48 (s, 1H), 7.37 (d, J=9.04 Hz, 2H), 7.08 (s, 1H), 6.65 (d, J=9.01 Hz, 2H), 4.59 (s, 2H), 4.38 (s, 2H), 3.77 (s, 3H), 3.71 (s, 3H), 3.57 (t, J=6.60 Hz, 2H), 3.39 (t, J=6.65 Hz, 2H). ¹³C NMR (100 MHz, d₄-MeOH): δ 151.8, 148.5, 142.2, 138.9, 138.3, 135.2, 133.1, 127.7, 126.2, 126.1, 121.1, 119.5, 114.1, 100.9, 54.5, 51.0, 48.3, 45.5, 34.8, 34.6. HRMS calcd for C₂₄H₂₇N₈O₂S⁺: 491.1978. found 491.2003. Retention time for analytical rpHPLC: condition (I) 11.13, (II) 14.43 minutes.

1-Methyl-1H-imidazole-4-sulfonic acid (3-cyano-benzyl)-{2-[(4-cyano-phenyl)-(3-methyl-3H-imidazol-4-ylmethyl)-amino]-ethyl}-amidem-cyanobenzyl, (8ad, X=CN, R¹=m-Cyanobenzyl, R²=1-Methyl-1H-imidazole, R³=Methyl): Reaction of 7ad according to procedure C, Yield 67%. ¹H NMR (400 MHz, d₄-MeOH): δ 8.88 (s, 1H), 7.82 (s, 1H), 7.80 (s, 1H), 7.64-7.60 (m, 3H), 7.48 (d, J=7.93 Hz, 1H), 7.44 (d, J=8.95 Hz, 2H), 7.16 (s, 1H), 6.67 (d, J=8.95 Hz, 2H), 4.61 (s, 2H), 4.34 (s, 2H), 3.86 (s, 3H), 3.80 (s, 3H), 3.57 (t, J=6.67 Hz, 2H), 3.44 (t, J=6.67 Hz, 2H). ¹³C NMR (100 MHz, d₄-MeOH): δ 151.4, 141.7, 139.9, 138.7, 137.8, 134.7, 133.5, 132.8, 132.7, 130.9, 127.1, 120.8, 119.4, 119.0, 113.6, 113.5, 100.4, 54.4, 50.8, 47.3, 45.2, 34.4, 34.3. □RMS (ESI): m/z calcd for C₂₆H₂₆N₈O₂SH⁺ 515.1978. found 515.1971. Retention time for analytical rpHPLC: condition (I) 13.78, (II) 18.94 minutes.

1-Methyl-1H-imidazole-4-sulfonic acid (4-cyano-benzyl)-{2-[(4-cyano-phenyl)-(3-methyl-3H-imidazol-4-ylmethyl)-amino]-ethyl}-amide (8ae, X=CN, R¹=p-Cyanobenzyl, R²=1-Methyl-1H-imidazole, R³=Methyl): Reaction of 7ae according to procedure C, Yield 60%. ¹H NMR (400 MHz, d₄-MeOH): δ 8.88 (s, 1H), 7.82 (s, 1H), 7.80 (s, 1H), 7.61 (d, J=8.22 Hz, 2H), 7.48 (d, J=8.22 Hz, 2H), 7.42 (d, J=9.01 Hz, 2H), 7.15 (s, 1H), 6.64 (d, J=9.01 Hz, 2H), 4.63 (s, 2H), 4.35 (s, 2H), 3.86 (s, 3H), 3.80 (s, 3H), 3.57 (t, J=6.72H, 2H), 3.42 (t, J=6.72 Hz, 2H). ¹³C NMR (100 MHz, d₄-MeOH): [symbol] 151.3, 143.7, 141.7, 138.5, 137.8, 134.6, 133.5, 130.8, 127.1, 120.8, 119.4, 119.0, 113.6, 112.9, 100.2, 54.9, 50.8, 47.5, 45.2, 34.4, 34.2. HRMS (ESI): m/z calcd for C₂₆H₂₆N₈O₂SH⁺ 515.1978. found 515.1970. Retention time for analytical rpHPLC: condition (I) 13.84, (II) 18.91 minutes.

1-Methyl-1H-imidazole-4-sulfonic acid biphenyl-3-ylmethyl-{2-[(4-cyano-phenyl)-(3-methyl-3H-imidazol-4-ylmethyl)-amino]-ethyl}-amide (8af, X=CN, R¹=Biphenyl-3-ylmethyl, R²=4-methyl-1H-imidazole, R³=Methyl): Reaction of 7af according to procedure C, Yield 69%. ¹H NMR (400 MHz, d₄-MeOH): δ 8.71 (s, 1H), 7.72 (s, 2H), 7.50 (m, 4H), 7.38 (m, 2H), 7.31 (m, 2H), 7.23 (d, J=7.72 Hz, 1H), 7.20 (d, J=9.04 Hz, 2H), 7.03 (s, 1H), 6.48 (d, J=9.09 Hz, 2H), 4.40 (s, 2H), 4.26 (s, 2H), 3.70 (s, 3H), 3.63 (s, 3H), 3.39 (m, 2H), 3.34 (m, 2H). ¹³C NMR (100 MHz, d₄-MeOH): δ 151.8, 143.1, 142.0, 141.9, 139.4, 138.8, 138.1, 135.0, 133.0, 130.9, 130.5, 129.8, 129.3, 129.2, 128.3, 128.1, 127.3, 121.2, 119.4, 113.9, 100.5, 55.7, 51.5, 47.1, 45.4, 34.8, 34.5. HRMS calcd for C₃₁H₃₂N₇O₂S⁺: 566.2338. found 566.2321. Retention time for analytical rpHPLC: condition (I) 14.51, (II) 24.77 minutes.

1-Methyl-1H-imidazole-4-sulfonic acid biphenyl-4-ylmethyl-{2-[(4-cyano-phenyl)-(3-methyl-3H-imidazol-4-ylmethyl)-amino]-ethyl}-amide (8ag, X=CN, R¹=Biphenyl-4-ylmethyl, R²=4-Methyl-1H-imidazole, R³=Methyl): Reaction of 7ag according to procedure C, Yield 74%. ¹H NMR (500 MHz, d₄-MeOH): δ 8.77 (s, 1H), 7.80 (s, 1H), 7.77 (s, 1H), 7.58 (dd, J=1.28, 8.50 Hz, 2H), 7.56 (d, J=8.23 Hz, 2H), 7.44 (t, J=7.47 Hz, 2H), 7.37 (d, J=8.21 Hz, 2H), 7.36 (tt, J=1.14, 7.38 Hz, 1H), 7.29 (d, J=9.05 Hz, 2H), 7.12 (s, 1H), 6.53 (d, J=9.10 Hz, 2H), 4.54 (s, 2H), 4.26 (s, 2H), 3.78 (s, 3H), 3.76 (s, 3H), 3.48 (m, 2H), 3.38 (m, 2H). ¹³C NMR (125 MHz, d₄-MeOH): δ 149.1, 139.9, 139.2, 139.0, 136.5, 135.4, 134.3, 132.3, 130.4, 128.7, 127.8, 126.4, 125.8, 125.5, 124.6, 118.4, 116.7, 111.2, 97.8, 52.8, 48.8, 44.4, 42.8, 32.0, 31.8. HRMS calcd for C₃₁H₃₂N₇O₂S⁺: 566.2338. found 566.2358. Retention time for analytical rpHPLC: condition (I) 15.69, (II) 30.13 minutes.

1-Methyl-1H-imidazole-4-sulfonic acid {2-[(4-cyano-phenyl)-(3-methyl-3H-imidazol-4-ylmethyl)-amino]-ethyl}-(4-pyrrol-1-yl-benzyl)-amide (8ai, X=CN, R¹=4-Pyrrol-1-yl-benzyl, R²=4-methyl-1H-imidazole, R³=Methyl): Reaction of 7ai according to procedure C, Yield 54%. ¹H NMR (400 MHz, d₄-MeOH): δ 8.72 (s, 1H), 7.74 (m, 4H), 7.49 (s, 1H), 7.32 (m, 4H), 7.13 (s, 1H), 7.06 (s, 1H), 6.52 (m, 3H), 6.24 (t, J=2.54 Hz, 2H), 4.49 (s, 2H), 4.12 (s, 2H), 3.74 (s, 3H), 3.72 (s, 3H), 3.40 (m, 2H), 3.32 (m, 2H). ¹³C NMR (100 MHz, d₄-MeOH): δ 151.4, 144.4, 138.4, 136.7, 135.3, 133.9, 129.5, 127.5, 121.2, 121.0, 120.2, 119.2, 114.7, 112.4, 111.4, 101.8, 52.5, 51.4, 47.7, 46.2, 34.7, 34.5. HRMS calcd for C₂₉H₃₁N₈O₂S⁺: 555.2285. found 555.2292. Retention time for analytical rpHPLC: condition (I) 13.21, (II) 20.55 minutes.

1-Methyl-1H-imidazole-4-sulfonic acid {2-[(4-cyano-phenyl)-(3-methyl-3H-imidazol-4-ylmethyl)-amino]-ethyl}-amide (6a, X=CN, R²=1-Methyl-1H-imidazole, R³=Methyl): Reaction of 5c according to procedure C after deprotection of Boc group. Yield 82%. ¹H NMR (400 MHz, d₄-MeOH): δ 8.89 (s, 1H), 7.78 (s, 1H), 7.67 (s, 1H), 7.52 (d, J=9.02 Hz, 2H), 7.24 (s, 1H), 6.91 (d, J=9.02 Hz, 2H), 4.83 (s, 2H), 3.90 (s, 3H), 3.75 (s, 3H), 3.66 (t, J=6.31 Hz, 2H), 3.21 (t, J=6.31 Hz, 2H). ¹³C NMR (100 MHz, d₄-MeOH): δ 151.8, 141.2, 140.4, 137.7, 130.0, 125.9, 120.9, 119.1, 114.0, 100.2, 51.5, 46.0 41.2 34.4, 34.3.

N-(4-Bromo-phenyl)-N-(3-methyl-3H-imidazol-4-ylmethyl)-N′-pyridin-2-ylmethyl-ethane-1,2-diamine (14, X=Br, R¹=2-Pyridylmethyl, R³=Methyl): Reaction of 5b according to procedure A, Yield 52%. ¹H NMR (400 MHz, d₄-MeOH): δ 8.78 (s, 1H), 8.46 (d, J=4.86 Hz, 1H), 7.73 (dt, J=7.70, 1.63 Hz, 1H), 7.31 (d, J=7.83 Hz, 1H), 7.28 (dd, J=5.03, 7.55 Hz, 1H), 7.25 (d, J=9.07 Hz, 2H), 7.14 (s, 1H), 6.78 (d, J=9.00 Hz, 2H), 4.59 (s, 2H), 4.30 (s, 2H), 3.74 (s, 3H), 3.66 (t, J=7.06 Hz, 2H), 3.22 (t, J=7.10 Hz, 2H). ¹³C NMR (100 MHz, d₄-MeOH): δ 151.3, 149.5, 146.2, 137.9, 136.7, 132.4, 132.0, 124.1, 123.2, 118.3, 117.3, 112.1, 50.9, 47.1, 45.4, 44.2, 33.0.

1-Methyl-1H-imidazole-4-sulfonic acid {2-[(4-bromo-phenyl)-(3-methyl-3H-imidazol-4-ylmethyl)-amino]-ethyl}-pyridin-2-ylmethyl-amide (13, X=Br, R¹=2-Pyridylmethyl, R²=4-methyl-1H-imidazole, R³=Methyl): Reaction of 14 according to procedure C, Yield 24%. ¹H NMR (400 MHz, d₄-MeOH): δ 8.75 (s, 1H), 8.42 (d, J=5.18 Hz, 1H), 7.88 (td, J=1.69, 7.76 Hz, 1H), 7.71 (s, 1H), 7.67 (s, 1H), 7.54 (d, J=7.93 Hz, 1H), 7.39 (dd, J=5.23, 7.51 Hz, 1H), 7.15 (d, J=9.11 Hz, 2H), 7.12 (s, 1H), 6.53 (d, J=9.14 Hz, 2H), 4.49 (s, 2H), 4.43 (s, 2H), 3.75 (s, 3H), 3.68 (s, 3H), 3.41 (m, 2H), 3.35 (m, 2H). ¹³C NMR (100 MHz, d₄-MeOH): δ 157.3, 148.2, 147.9, 142.0, 141.7, 139.1, 138.0, 133.7, 133.5, 127.5, 126.3, 125.7, 119.7, 116.9, 111.7, 55.3, 51.3, 48.1, 45.8, 34.8, 34.6. HRMS calcd for C₂₃H₂₇N₇O₂S⁺: 544.1130. found 544.1145. Retention time for analytical rpHPLC: condition (I) 11.21, (II) 14.74 minutes.

Plasmodium strains. The P. falciparum strains used in this study were 3D7 (Netherlands, sensitive) provided by Dr. Pradipsinh Rathod from the University of Washington and K1 (Thailand, ChQ-R, Pyr-R) obtained from the MR4Unit of the American Type Culture Collection (ATCC, Manassas, Va.).

P. falciparum culture. Strains of P. falciparum were sustained in vitro based on experimental techniques as described by Trager and Jensen.²⁶ Cultures were maintained in RPMI-1640 (Sigma, St. Louis, Mich.) with 2 mM L-glutamine, 25 mM HEPES, 33 mM NaHCO₃, 20 μg/ml gentamicin sulfate and 20% (v/v) heat-inactivated human plasma type A+ (R^{P}-20P). Type A+ erythrocytes were obtained from lab donors, washed three times with RPMI, re-suspended in 50% RPMI, and stored at 4° C. Parasites were grown in 10 mL of a 2% hematocrit/RP-20P (v/v) in 50-mL flasks under a 5% CO₂, 5% O₂, and 90% N₂ atmosphere.

P. falciparum ED₅₀ determination. One □L of PFTI dissolved in DMSO was added to each well of a 96-well plate followed by the addition of 200 μL of P. falciparum culture at parasitemia and hematocrit of 0.5%. Plates were flushed with 5% CO₂, 5% O₂, and 90% N₂ then incubated at 37° C. for 48 hr. [8-³H]-hypoxanthine (0.3 μCi, 20 Ci/mmol, American Radiolabeled Chemicals) in 30 μL RP-20P was added to cultures and incubated for an additional 24 hours. Cells were harvested onto filter mats by a Multiharvester (Skatron, Sunnyvale, Calif.) and the radioactivity incorporated into the parasites was counted on a β-scintillation counter. The background level detected with uninfected erythrocytes was subtracted from the data. The ³H-incorporation into infected RBCs with 1 μL DMSO vehicle alone represents 100% malaria growth. ED₅₀ values were determined by linear regression analysis of the plots of ³H-hypoxanthine incorporation versus concentration of compound.

PfPFT IC₅₀ determination. The PFT assay used to determine the IC₅₀s of the compounds is based on a scintillation proximity assay.¹² Assays were carried out in 30 mM potassium phosphate pH 7.7, with 5 mM DTT, 0.5 mM MgCl₂, 20 μM ZnCl₂, 0.3 μCi (0.75 μM) [³H]farnesyl pyrophosphate (15 μCi/mmol, American Radiolabeled Chemicals Inc.), 1 μM RAS-CVIM protein substrate in a total volume 20 μL which included 1 μL of PFTI solution in DMSO and 3 μL of partially purified PfPFT.¹² Assays in the absence of PFTI and PfPFT were included as positive and negative controls, respectively. Reaction mixtures were incubated at 30° C. for 30 minutes and terminated by addition of 200 μL of 10% HCl/ethanol. After overnight incubation at room temperature, the mixtures were filtered onto a Whatman glass fiber filter (VWR, San Francisco, Calif.) using a 96-well vacuum manifold. After washing with 100% ethanol, the filter was cut, and individual slices were counted in a beta-scintillation counter. IC₅₀ values were calculated using linear regression analysis of the plots of ³H-FPP prenylation versus concentration of compounds.

Microsome metabolism. Liver microsome metabolism assays were performed with female pooled microsomes from BD Biosciences (20 mg/mL). Reaction wells containing: phosphate buffer (232 μL, 0.6 M), MgCl₂ (12.0 μL, 0.1 M), EDTA (0.8 μL, 0.5 M), 10x NADPH regenerating system (40.0 μL), Glucoide-6-phosphate deghydrogenase (0.8 μL, 500 U/mL), milliq water (267.8 μL), and liver microsomes (10.0 μL, 20 mg/mL) were heated at 37° C. for 10 mins. To each reaction well was added inhibitor (200 μM, 2.0 μL) in DMSO. Reaction vessels were quenched with acetonitrile (75 μL) and internal standard at designated time points, and the sample immediately frozen (−20° C.). Metabolites and unreacted inhibitor were quantified by mass spectral analysis.

### Results and Discussion

Design. PFTase is one of three closely related heterodimeric zinc metalloenzymes (protein farnesyl- and geranylgeranyl transferases I and II), that catalyze the transfer of prenyl groups from farnesyl or geranylgeranyl pyrophosphate, to the free thiol of a cysteine residue within a tetrapeptide recognition sequence (CaaX, a=aliphatic amino acid, X often is M, S, A, or Q for PFTase) located at the carboxyl terminus of the substrate protein.¹⁶ The X-ray crystal structure of rat PFT complexed with the seleno-tetrapeptide Ac-Cys-Val-Ile-Met(Se)-OH and a farnesylpyrophosphate (FPP) analogue (1JCR), shows a heterodimeric zinc metalloenzyme composed of a 48 kDa α-subunit and a 46 kDa β-subunit, with the tetrapeptide in an extended conformation and coordinated to the catalytic zinc ion through the cysteine thiol, in close proximity to the FPP phosphonate. The sequences of the two subunits of PfPFT were obtained from the PlasmoDB database (Gene loci: PFL2050w, α, and chr11.glm_{—}528, β),^{17,18} and aligned with the reported structure of rat PFT using the program T-COFFEE.¹⁹ While PfPFT is found to be considerably different to rat PFTase, being significantly larger in both the α- (472 us 379 residues) and β-subunits (621 vs 437), the differences are mainly due to insertions in the PfPFT protein sequence, and overall there is minimal difference in the residues that form the active site.

A homology model of Plasmodium farnesyltransferase (PfPFT) was generated with MODELLER,¹²⁰ using the sequence alignment of PfPFT on the template crystal structure of rat PFT (1JCR). Only regions with reasonable reliability of the alignment were included. The model of PfPFT comprises the following sequence segments (the residue numbers of the corresponding segments of the rat PFT subunits are given in parentheses): α: 72-164 (87-179), 300-411 (184-283); β: 421-677 (71-315), 806-896 (330-417). The levels of sequence identity (similarity) between PfPFT and rat PFT in these regions amount to 23% (53%) for the α- and 37% (56%) for the β-subunit. The catalytic zinc ion, six structurally conserved water molecules and FPP were included in the model. The conformation of FPP was considered flexible during the model calculations. For this purpose the force field parameters for FPP were added to the MODELLER force field on basis of the lipid-parameters of the charmm27 force field.²¹ The model with the lowest value of the objective function of MODELLER from twenty different calculations was used for docking studies.

The homology model indicates a large, open, and predominately hydrophobic cavity for the active site (˜20×20×20 Å), with the phospholipid binding partner (FPP) extending across the cavity base. The Zn ion coordinates to three residues (Cys 661, Asp 659 and His 838), with a water molecule hydrogen bonded between the terminal phosphate of FPP and Asp 659 defining the limit of the Zn binding domain. The remainder of the active site cavity includes two well defined hydrophobic pockets (Lys 149, Asn 317 Ser 150, Phe 150 and Trp 456 Trp 452, Tyr 837) and a larger hydrophilic domain formed by Arg 564, and three water molecules participating in a hydrogen bonded network between Ser 44 and Gln 152.

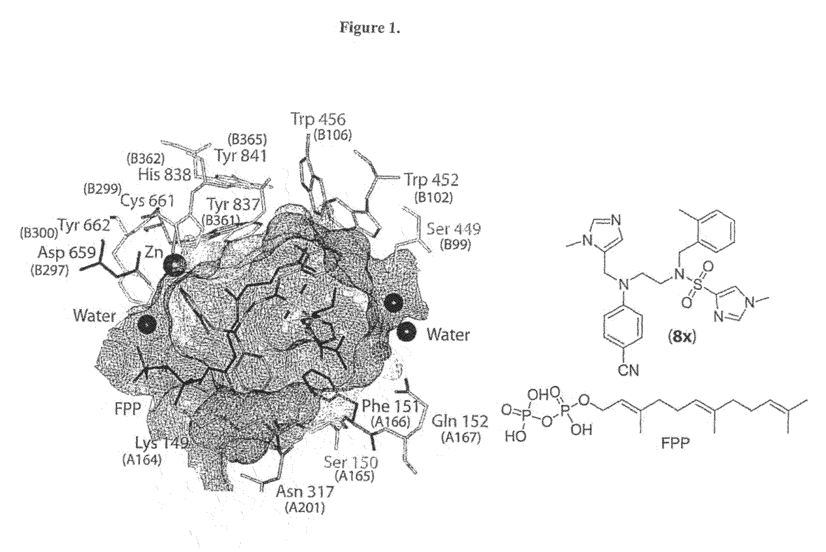

We envisaged accessing these four pockets from a simple aliphatic tether. Application of a flexible scaffold offers several advantages to the design of a new series of farnesyltransferase inhibitors. A simple acyclic scaffold may be obtained through a short series of straight forward chemical transformations, and may confer refractivity to resistance arising from mutation of PfPFT.²³ One of the simplest of scaffolds conceivable, ethylenediamine, affords an inexpensive, four fold substitutable flexible tether, of suitable size to project the appended diversity into the active site pockets. Imidazole provides a convenient zinc binding group, which has been consistently demonstrated to confer activity in other series of inhibitors. In order to maintain a suitably lipophilic compound, extension from the amines has been restricted to formation of anilines, sulfonamides, and amides. Flexible ligand docking studies (GOLD)²² of a series of compounds incorporating this basic design demonstrate complementarity to the active site of the homology model (FIG. 1).

Synthesis. An initial series of farnesyltransferase inhibitors was prepared as outlined in Scheme 1, FIG. 2, through a simple series of reductive amination, amide coupling, reduction and alkylation. This route is not effective for the preparation of compounds bearing strong para election withdrawing groups on aniline. In these systems the amide coupling become increasingly difficult, and in the case of 4-cyanoaniline, the selective reduction of the amide also proves problematic. Performing the reductive amination with aniline derivatives (Br, CN, Ph) and Boc-glycinal, followed by alkylation of the resulting aniline with chloromethyl-N-methylimidazole proved suitable for the formation of the 4-bromo and 4-phenyl derivatives, however reductive amination with 4-cyanoaniline proceeded in poor yield. For the nitrile system, excellent yields of aniline 4c can be obtained via nucleophilic substitution of para-fluorobenzonitrile with mono protected ethylenediamine. Chemoselective alkylation of aniline 4c, via double deprotonation with LDA and subsequent alkylation at the anilide anion proceeds smoothly in THF, with no evidence of alkylation of the carbamate (FIG. 3). Finally, alkylation of the carbamate, deprotection and coupling to sulfonyl or acid chlorides furnishes the desired series of inhibitors.

Structure-Activity Relationships. An initial series of inhibitors maintained the zinc binding imidazole and hydrophobic components (R¹=benzyl, X=H) constant, while exploring a small focused diversity set (R², 7 substitutions) of sulfonamide substitutions predicted by docking studies (GOLD)²² to reasonably access the hydrophilic pocket. The inhibition of PfPFT for these preliminary inhibitors was determined at a single concentration (50 nM), using a scintillation proximity assay with partially purified PfPFT (Table 1).¹² Four of the seven compounds inhibited PfPFT at 50 nM, with one compound (1-methyl-1H-imidazole-4-sulfonamide, 3c) demonstrating very promising inhibition (32% at 50 nM). Elaboration of 3c, through incorporation of para-substituted anilines (Br, CN, Ph, Scheme 2), induced at least a two fold improvement in activity (compare 8a and 3c, Table 2). Inspection of the docked conformations of 8a and 8c indicate the para position of the aniline can reasonably access a hydrophilic domain formed by Ser 150, Asn 317, and Lys 149, at the limit of the mostly hydrophobic pocket occupied by the aniline (FIG. 1).

TABLE 1
SAR of R² Sulfonamide Substitutions.

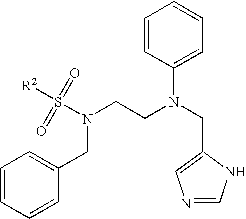

| | |
|---|---|
| Compound | % Inhibition at 50 nM |

| Number | R² | PfPFT |
|---|---|---|
| 3a | 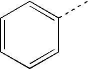 | 7 |
| 3b | 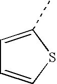 | 5 |
| 3c | 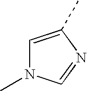 | 32 |
| 3d | 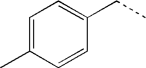 | 3 |
| 3e | 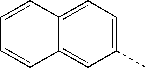 | 0 |
| 3f | 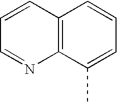 | 0 |
| 3g | 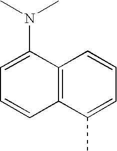 | 0 |

Modification of the zinc binding imidazole has previously lead to a significant impact on inhibitor potency in related systems,¹³ and appears particularly important for effective activity in whole cells. The homology model for PfPFT indicated that small alkyl groups appended to the imidazole (3-methyl-3H-imidazol-) might reasonably be accommodated, and the corresponding methyl imidazole inhibitors demonstrated significantly improved activity (compare 8b to 8a, Table 2). Inhibitor 8b, and related methyl imidazole inhibitor 8d, additionally demonstrated inhibition of the growth of parasites in whole cells (3D7, K1: ED₅₀<1 μM), as monitored through incorporation of tritium labeled hypoxanthine (see experimental for details).

TABLE 2
SAR of X and R³ Substitutions.

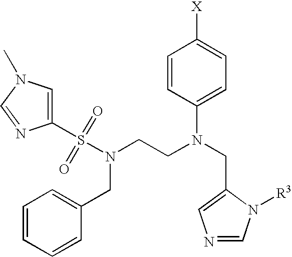

| Compound | | | % Inhibition of PfPFT | | ED₅₀ (nM)^{a} | |
|---|---|---|---|---|---|---|
| Number | X | R³ | at 50 nM | at 5 nM | 3D7 | K1 |
| 3a | H | H | 32 | | | |
| 8a | Br | H | 80 | | | |
| 8b | Br | Me | 95 | 86 | 675 | 3200 |
| 8c^{b} | Ph | Me | 85 | 49 | 2500 | 2600 |
| 8d | CN | Me | 98 | 86 | 349 | 367 |

^{a}Inhibitor concentration required to decrease hypoxanthine incorporation into parasites twofold.
^{b}2-Methylbenzyl in place of benzyl (see Table 3, R¹).

Having identified a potent inhibitor with good whole cell activity (8d), the series of sulfonamides was revisited; now incorporating both methyl imidazole, and para-cyano aniline (Table 3). Inclusion of 1-methyl-1H-imidazole-4-sulfonamide as the R² substituent again conferred the best in-vitro activity against PfPFT (8d, 86% inhibition at 5 nM), with similar potency observed for 2-pyridyl sulfonamide (8g, 64% inhibition at 5 nM). Phenyl, thiophene or quinoline sulfonamides were less active. Replacement of the sulfonamide by small alkyl amides (8j methyl, and 8k isopropyl), indicated by docking studies to be able to access a hydrophobic cleft inaccessible to the larger sulfonamides, resulted in significantly reduced activity (56 and 61% inhibition at 50 nM respectively). Good inhibition of the growth of parasites in whole cells (3D7, K1) was observed with methyl-1H-imidazole-4-sulfonamide (8d, ED₅₀: 349 and 375 nM: respectively), and dansyl sulfonamide (8i, ED₅₀: 300 and 388 nM) despite the lower PfPFT activity observed for 8i.

TABLE 3
SAR of R² Substitutions with X = CN.

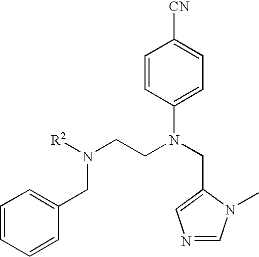

| | | % Inhibition | | | |
|---|---|---|---|---|---|
| Compound | | of PfPFT | | | |
| Num- | | at 50 | at 5 | ED₅₀ (nM) | |
| ber | R² | nM | nM | 3D7 | K1 |
| 8e | 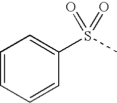 | 69 | 18 | 2500 | 2250 |
| 8f | 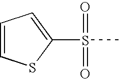 | 74 | 17 | 550 | 1125 |
| 8d | 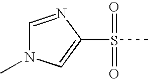 | 98 | 86 | 349 | 375 |
| 8g | 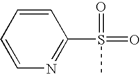 | 95 | 64 | 570 | 2300 |
| 8h | 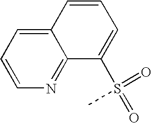 | 35 | 14 | 2100 | 2400 |
| 8i | 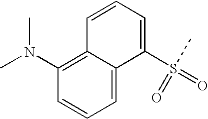 | 29 | 6 | 300 | 388 |
| 8j | 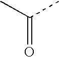 | 56 | 0 | 2500 | >5000 |
| 8k | 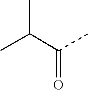 | 61 | 0 | 2000 | >5000 |

A broader series of substitutions was investigated for the R¹ hydrophobic pocket (˜20 compounds), while maintaining the remaining three groups as previously optimized. Replacement of the benzyl substituent with small alkyl groups that retained sp² hybridized centers (propenyl, methylpropenyl, bromopropenyl and ^{t}butylcarbamate) provided inhibitors with reduced activity against PfPFT, and no inhibition against the growth of parasites in cells (>5000 nM). Introduction of cyclohexylmethyl in 8q as a close comparison for benzyl, provided similar activity to the parent inhibitor 8d, and offers slight improvement in whole cell activity (ED₅₀: 3D7 220 nM, K1 850 nM). Incorporation of oxygen into the cyclohexyl ring to afford the tetrahydropyran 8r is tolerated, while similar piperidine 8s is inactive. Activity of the piperidine derivatives is recovered with amides 8t to 8v, with larger amides conferring improved activity. The large tert-butoxy carbamate derivative 8v provides similar in-vitro inhibition as the parent benzyl derivative 8d, but is significantly more toxic to erythrocytic parasite growth (ED₅₀: 3D7 88 nM, K1 54 nM). The isoelectronic inhibitor 8w is similarly active against both PfPFT, and parasite growth in whole cells.

TABLE 4
SAR R1 Substitutions x = CN

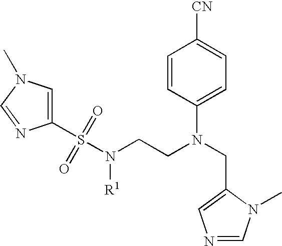

| Compound | | % Inhibition of PfPFT | | ED₅₀ (nM) | |
|---|---|---|---|---|---|
| Number | R¹ | at 50 nM | at 5 nM | 3D7 | K1 |
| 8l | 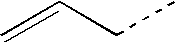 | 42 | 1 | >5000 | >5000 |
| 8m | 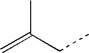 | 77 | 6 | >5000 | >5000 |
| 8n | 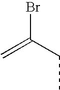 | 80 | 25 | >5000 | >5000 |
| 8o | 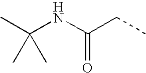 | 6 | 4 | 2800 | >5000 |
| 8p | 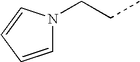 | 56 | 11 | >5000 | >5000 |
| 8q | 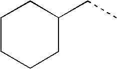 | 93 | 63 | 220 | 850 |
| 8r | 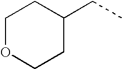 | 84 | 28 | 575 | 400 |
| 8s | 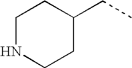 | 5 | 5 | >5000 | >5000 |
| 8t | 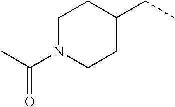 | 44 | 8 | 5000 | 3500 |
| 8u | 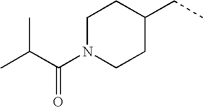 | 92 | 57 | 230 | 180 |
| 8v | 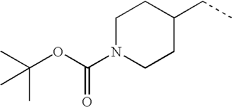 | 96 | 81 | 88 | 54 |
| 8w | 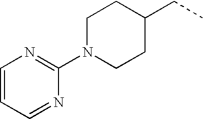 | 96 | 74 | 130 | 85 |
| 8d | 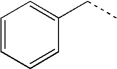 | 98 | 86 | 349 | 375 |
| 8x | 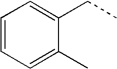 | 99 | 95 | 93 | 150 |
| 8y | 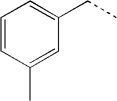 | 94 | 61 | 300 | 1000 |
| 8z | 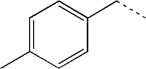 | 54 | 45 | 2700 | >5000 |
| 8aa | 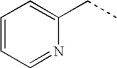 | 93 | 64 | 750 | 1000 |
| 8ab | 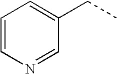 | 74 | 18 | 2800 | 4250 |
| 8ac | 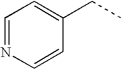 | 62 | 22 | 3700 | 5000 |
| 8ad | 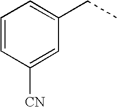 | 89 | 45 | 350 | 1800 |
| 8ae | 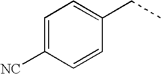 | 48 | 24 | 1600 | 4100 |
| 8af | 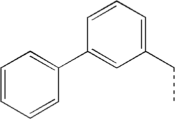 | 93 | 56 | 2400 | 3250 |
| 8ag | 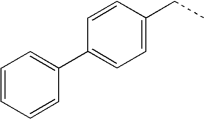 | 19 | 10 | 700 | 2500 |
| 8ah | 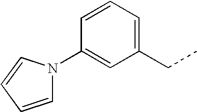 | 74 | 37 | 2500 | 4000 |
| 8ai | 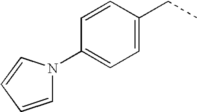 | 85 | 39 | 257 | 410 |

Incorporation of nitrogen in aryl substitutents (pyridine) is tolerated ortho, but not meta or para, and in all cases diminishes toxicity to parasites. A similar trend is observed for methyl and cyano substitution, with 2-methylbenzyl derivative 8x providing excellent inhibition against both PfPFT and parasite growth in cells. Condensation of both 2-pyridyl (7aa) and 2-cyano-derivatives with 1-methyl-1H-imidazole-4-sulfonyl chloride were problematic, and the later was not prepared. Large R¹ benzyl substituents (phenyl or pyrrol) can be accommodated at the meta or para positions with a modest reduction of PfPFT inhibition. In both cases whole cell activity was better for the para substituted compounds (8ag, 8ai), with para pyrrol displaying comparable whole cell activity to the unsubstituted benzyl system. The 4-phenylbenzyl derivative 8ag is approximately four fold less active then the closely related pyrimidine 8w, despite their similar structures, while the related 4-pyrrole inhibitor 8ai displays intermediate activity.

Overall, minor structural modification of any of the four ethandiamine substituents has significant impact on PfPFT activity, and generally good correlation between PfPFT inhibition and toxicity to cultured parasites is observed, supporting PfPFT as the relevant target for the observed anti-malarial activity. The generally straightforward synthesis of these structurally simple inhibitors facilitates rapid incorporation of diversity at any position, and should greatly ease elucidation of inhibitors with ‘drug like’ pharmacokinetics.

Selectivity. Previously reported PFT inhibitors have either shown no selectivity for inhibition of parasitic over mammalian farnesyltransferase, or are highly selective inhibitors of the mammalian enzyme.^{7,8,12} While inhibition of farnesyltransferase has been demonstrated to have limited toxicity to mammalian cells at concentrations required to elicit a therapeutic response,⁸ selective inhibition of parasitic farnesyltransferase may yet be an essential goal in development of safe and effective anti-malarial PFT inhibitors. To examine the selectivity of this new series of farnesyltransferase inhibitors, twelve compounds were selected on the basis of structural diversity and in-vitro PfPFT activity, and their 50% inhibition concentrations (IC₅₀) against both Plasmodium and rat farnesyltransferase determined (Table 5). Eleven of the twelve compounds demonstrated selectivity for inhibition of Plasmodium over rat farnesyltransferase, representing to our knowledge the first reported series of Plasmodium selective farnesyltransferase inhibitors. Six inhibitors displayed better than ten-fold selectivity for Plasmodium (13, 8aa, 8c, 8d, 8b, 8x), with inhibitors 8b and 8c displaying over 100 fold selectivity. Examination of low energy docked conformations (GOLD)²² of 8b and 8c in our homology model, shows a consistent position of the para aniline substituent into the pocket formed by Asn 317 and Phe 151, corresponding to two (His 201 and Tyr 166 respectively) of only three residues in the active site that differ between rat (pdb: 1JCR) and PfPFT. Further work is being undertaken to better elucidate the binding modes of this series of inhibitors, but these preliminary observations provide tantalizing evidence for the potential development of highly selective PfPFT inhibitors, through exploitation of the modest active site structural differences of farnesyltransferase isoforms.

TABLE 5
Comparison of inhibitor activity against Plasmodium falciparum and Rat FTase.

| | Compound | | | IC₅₀ (nM)^{a} | | |
|---|---|---|---|---|---|---|
| | X | R¹ | R² | PfPFT | Rat | Selectivity^{b} |
| 8d | CN | 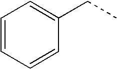 | 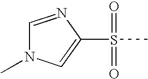 | 0.5 | 25 | 47 |
| 8b | Br | 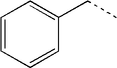 | 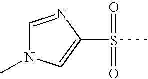 | 2.0 | 290 | 145 |
| 13 | Br | 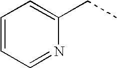 | 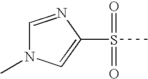 | 1.9 | 83 | 44 |
| 8aa | CN | 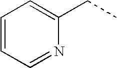 | 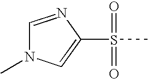 | 2.1 | 43 | 20 |
| 8x | CN | 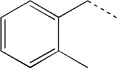 | 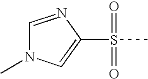 | 0.6 | 15.5 | 27 |
| 8c | Ph | 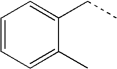 | 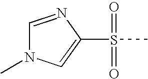 | 8.0 | >1000 | >125 |
| 8e | CN | 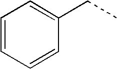 | 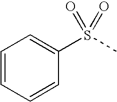 | 8.9 | 13 | 1.5 |
| 8i | CN | 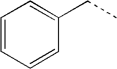 | 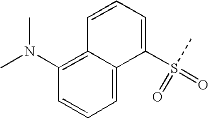 | 55 | 180 | 3.3 |
| 8q | CN | 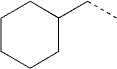 | 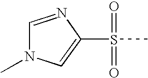 | 4.5 | 7.5 | 1.7 |
| 8ag | CN | 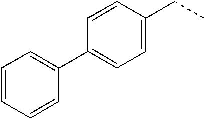 | 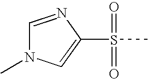 | 240 | 880 | 3.7 |
| 8o | CN | 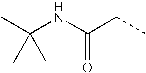 | 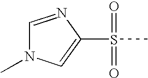 | >1000 | >1000 | |
| 8ae | CN | 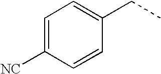 | 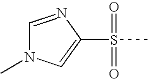 | 95 | 350 | 3.7 |

^{a}Inhibitor concentration required to decrease transferase activity twofold.
^{b}Ratio of rat to Plasmodium PFT activity.

Pharmacokinetics. On the basis of their in-vivo activity and structural diversity, eleven PfPFT inhibitors were evaluated for metabolism and absorption, and in five cases oral bioavailability in mice or rats was examined (Table 6). Three of the eleven inhibitors are structurally divergent at aniline (8d, 8b, 8c X=CN, Br, Ph respectively), seven represent modifications of the hydrophobic binding substituent R¹, and one inhibitor differs by the hydrophilic binding sulfonamide R² (8g). For each of these inhibitors, the concentration required to reduce PfPFT activity by 50% (IC₅₀) has also been determined (Table 6).

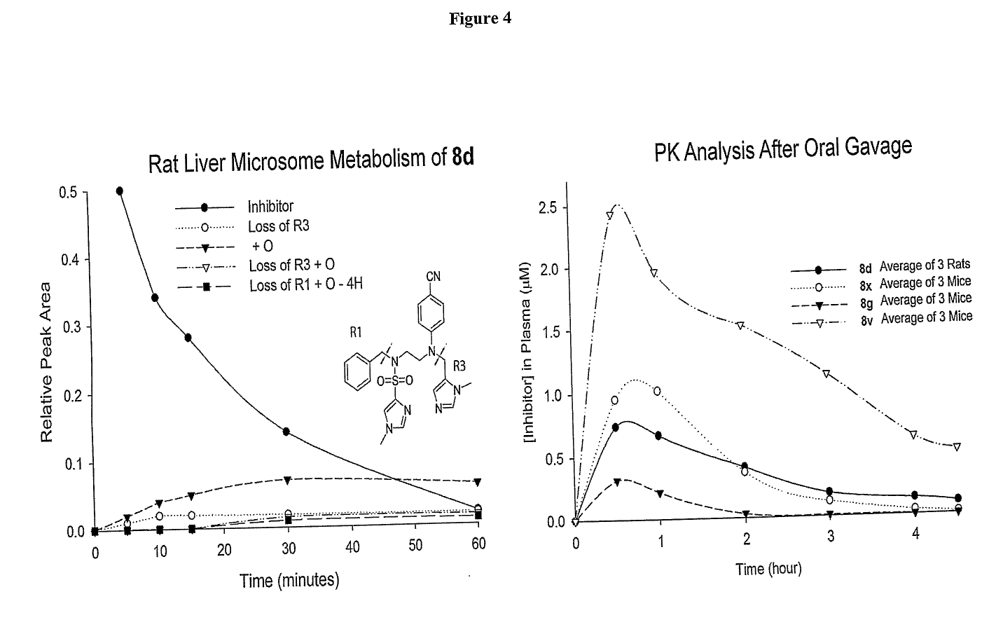

Metabolism. Liver microsomes provide a convenient model of in-vitro hepatic metabolism, allowing rapid initial assessment of the relative metabolic stability of a series of inhibitors. On treatment of 8d with rat or mouse liver microsomes, complete metabolism was observed within one hour, with inhibitor half lives of 18 and 9 minutes respectively (FIG. 4). Residual metabolite mass spectrum ionization reflected only a minor proportion of the initial ionization observed (<10%), and corresponded principally to oxidation of the inhibitor (+O), and oxidation with loss of the aniline imidazole (—R³+O). Despite the presence of the strong para electron withdrawing group (CN), a metabolism pathway may reasonably involve oxidation of the aniline nitrogen, followed by N-dealkylation. However, the metabolism half lives of inhibitors 8b and 8c, bearing less electronegative para-substitution (Br and Ph), exhibit only relatively minor charges in stability, suggesting aniline oxidation may not be the primary site of metabolism.

The methylbenzyl inhibitor 8x displayed a very similar metabolism profile to 8d, despite the additional benzylic position, which may reasonably be expected to be readily oxidized. In comparison 8q, incorporating cyclohexylmethyl as the hydrophobic substituent in place of the benzyl, is metabolized significantly more rapidly (t_{1/2}<5 mins). The related but larger piperidine derivatives 8v and 8w are considerably more stable (rat t₂=60, 40 mins respectively), which appears not to be directly related to the increased size of this substituent, given the rapid metabolism of the very similar 4-phenyl and 4-pyrolle benzyl derivatives 8ag and 8ai (mouse t_{1/2}<4 mins). Inhibitors 8v and 8w bearing isoelectronic R¹ piperidine substitution represent the most metabolically stable inhibitors observed in this series, and in general modification of the R¹ position has been found to have the greatest overall impact on the rate of microsome metabolism. Efforts are ongoing to identify the principal metabolic pathway operative for this series of inhibitors.

Absorption. Caco-2 cells cultured on a semi-permeable membrane form a highly functionalized epithelial barrier, with remarkable similarity to small intestinal epithelial cells, including high levels of brush border hydrolases and well developed junctional complexes. The apparent permeability of small molecules across these membranes represents a well established in-vitro model of in-vivo intestinal wall transport, that has demonstrated good correlation with intestinal absorption in humans.^{24,25} The apparent permeability coefficients of a selection of ethanediamines PFT inhibitors were generally low (Table 6, 0.4-1.5×10⁻⁶ cm/s), but not unacceptably removed from values typically observed for drugs that are fully absorbed in humans (>1×10⁻⁶ cm/s).²⁵ Sensitivity to structural modification was observed, with three compounds (8q, 8aa, 8g) displaying reasonable permeability coefficients (>1.5×10⁻⁶ cm/s).

Oral Bioavailability. Oral administration is the preferred route of drug delivery in general, but is essential when considering the development of an effective anti-malarial for use in the third world. Oral administration of 8d, an inhibitor with both moderate microsome stability and apparent permeability, in saline (12.5 mg in 90% saline, 3% ethanol, 7% tween) to rats with monitoring of inhibitor concentration in plasma over five hours identified a peak inhibitor concentration (Cₘₐₓ) of 0.74 μM after 30 minutes (Tₘₐₓ), with an elimination half life of 96 minutes (t_{1/2}) (Table 6, below). Similar inhibitor concentrations in plasma were observed for 8x after oral dosing in mice (1 mg), with a slightly elevated peak inhibitor concentration (Cₘₐₓ: 1.05 μM, Tₘₐₓ: 40 mins). On the basis of Caco-2 permeability and similar metabolism profiles to 8d and 8x, 8g (Caco-2: 2.8 cm·s⁻¹×10⁻⁶) was expected to demonstrate improved oral bioavailability. However, oral administration of 8g to mice identified extremely disappointing concentrations of the inhibitor in plasma, with a maximum peak concentrations and clearance rates (Cₘₐₓ: 0.74 μM, Tₘₐₓ: 30, t_{1/2}: 16 mins) significantly lower than those observed for 8d or 8x (Table 6). In comparison 8v, which demonstrated long half life stability against liver microsome metabolism (rat t_{1/2}: 60 mins), had considerably improved inhibitor availability in blood plasma after oral dosing in mice. An average concentration of 8v in plasma ˜6 fold above the concentration required to reduce parasite growth in erythrocytes by 50% (ED₅₀:3 D7 88 nM, K1 54 nM) was maintained for the duration of the experiment (5 hours), with an average peak plasma inhibitor concentration 30 fold over the ED₅₀ observed within 40 minutes (Table 6, below).

TABLE 6
In-Vitro, In-Vivo and Pharmacokinetic Properties of Selected PfPFT Inhibitors.

| | | IC₅₀ | | | CaCo | | Oral Availability in Mice^{c} | | |
|---|---|---|---|---|---|---|---|---|---|
| | | (nM)^{a} | ED₅₀ (nM)^{b} | | cm · s⁻¹ × | Microsome | AUC | Cₘₐₓ | t_{1/2} |
| | Structure | PfPFT | 3D7 | K1 | 10⁻⁶ | t_{1/2} (min) | (μM · min) | (μM) | (min) |
| 8d | 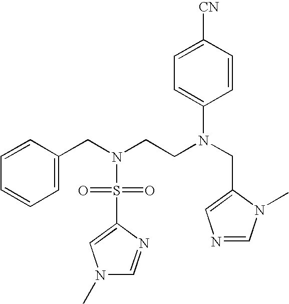 | 0.54 | 349 | 375 | 1.1 | Mouse: 9Rat: 18 | 112.0 | 0.74 | 95.9 |
| 8b | 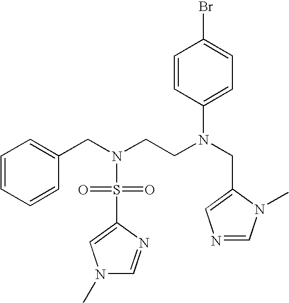 | 2.0 | 675 | 3200 | | Mouse: 2.9 | | | |
| 8c | 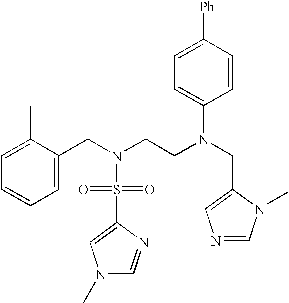 | 8 | 2600 | 2500 | 0.4 | Mouse: 9.4Rat: 9.5 | | | |
| 8x | 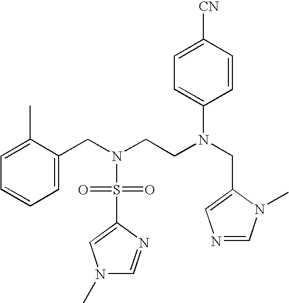 | 0.6 | 93 | 150 | 0.4 | Mouse: 8Rat: 20 | 103^{d} | 1.05^{d} | 32.6^{d} |
| 8q | 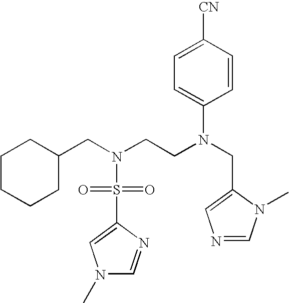 | 4.5 | 220 | 850 | 1.5 | Mouse: <5Rat: 4 | | | |
| 8aa | 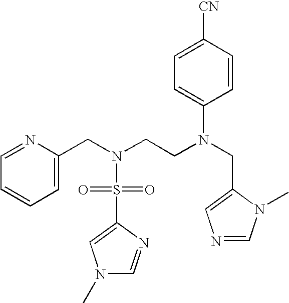 | 2.1 | 750 | 1000 | 1.8 | | | | |
| 8v | 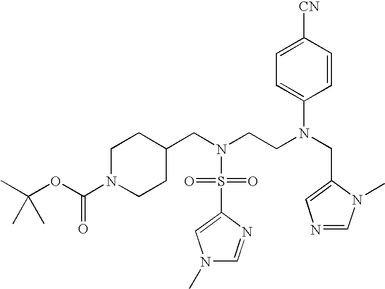 | 1.2 | 88 | 54 | 0.9 | Mouse: 14Rat: 60 | 414 | 2.97 | 70 |
| 8w | 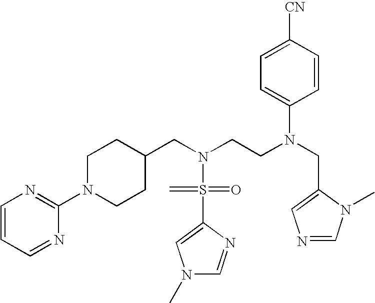 | 1.5 | 130 | 85 | | Mouse: 17Rat: 40 | | | |
| 8ag | 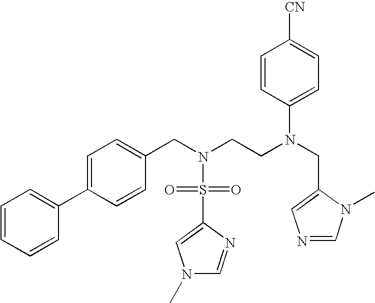 | 240 | 700 | 2500 | | Mouse: 3.0 | | | |
| 8ai | 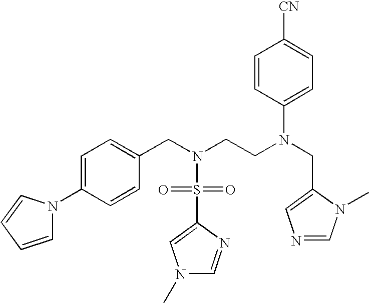 | 11 | 257 | 410 | | Mouse: 3.6 | | | |
| 8g | 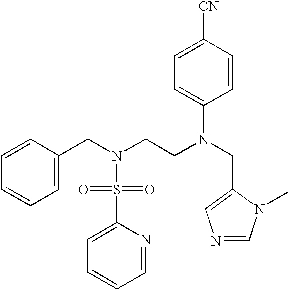 | 2.8 | 570 | 2300 | 2.8 | Mouse: 21Rat: 10.9 | 20.9 | 0.31 | 15.6 |

^{a}The concentration of inhibitor required to reduce PfPFT activity by 2-fold.
^{b}The concentration of inhibitor required to reduce hypoxanthine incorporation into parasites by 2-fold.
^{c}Average results for three mice.
^{b}Average results for thress rats.

### CONCLUSION

In summary, a new series of simple acyclic PfPFT inhibitors have been developed, and their efficacy evaluated against PfPFT, and reduction of parasite load in infected erythrocytes. Compounds based on this readily accessible scaffold are found to be highly active inhibitors of PfPFT, with IC₅₀ values as low as 0.5 nM identified from the initial diversity set (˜40 compounds). Effective translation of this activity into whole cell models of parisitemia are observed, with four compounds requiring doses of less than 100 nM to reduce parasite populations (3D7, K1) in erythrocytes by 50%. A preliminary study of the pharmacokinetic profile of this series of inhibitors identifies metabolism and absorption rates, as measured by microsome metabolism and Caco-2 permeability, to be responsive to minor structural modification. Relatively metabolically stable (t_{1/2} ˜60 mins) inhibitors have been identified, in addition to compounds with promising Caco-2 permeability. Further, oral gavage of a microsome stable inhibitor to mice identified very encouraging concentrations of inhibitor maintained in blood plasma over five hours. It is expected that elaboration of this inhibitor series will identity exceptionally potent inhibitors of PfPFT, with suitable pharmacokinetic profiles to allow a drug candidate to be taken into clinical trial. The structurally simplicity that underlines the design of these compounds should greatly facilitate third word nation access to any potential drug emergent from these novel PFT inhibitors.

### REFERENCES

(1) Sachs, J.; Malaney, P. The economic and social burden of malaria. Nature (London, United Kingdom) 2002, 415, 680-685.
(2) Olliaro, P. L.; Bloland, P. B. Clinical and public health implications of antimalarial drug resistance. Antimalarial Chemotherapy 2001, 65-83.
(3) Wiesner, J.; Ortmann, R.; Jomaa, H.; Schlitzer, M. New antimalarial drugs. Angewandte Chemie, International Edition 2003, 42, 5274-5293.
(4) May, J.; Meyer, C. G. Chemoresistance in falciparum malaria. Trends in Parasitology 2003, 19, 432-435.
(5) Ridley, R. G. Medical need, scientific opportunity and the drive for antimalarial drugs. Nature (London, United Kingdom) 2002, 415, 686-693.
(6) Rosenthal, P. J. Antimalarial drug discovery: old and new approaches. Journal of Experimental Biology 2003, 206, 3735-3744.
(7) Chakrabarti, D.; Azam, T.; DelVecchio, C.; Qiu, L.; Park, Y.-i. et al. Protein prenyl transferase activities of Plasmodium falciparum. Molecular and Biochemical Parasitology 1998, 94, 175-184.
(8) Nallan, L.; Bauer, K. D.; Bendale, P.; Rivas, K.; Yokoyama, K. et al. Protein Farnesyltransferase Inhibitors Exhibit Potent Antimalarial Activity. Journal of Medicinal Chemistry 2005, 48, 3704-3713.
(9) Clough, B.; Wilson, R. J. M. Antibiotics and plasmoidal plastid organelle. Antimalarial Chemotherapy 2001, 265-286.
(10) Loyevsky, M.; Gordeuk, V. R. Iron chelators. Antimalarial Chemotherapy 2001, 307-324.
(11) Gelb, M. H.; Van Voorhis, W. C.; Buckner, F. S.; Yokoyama, K.; Eastman, R. et al. Protein farnesyl and N-myristoyl transferases: piggy-back medicinal chemistry targets for the development of antitrypanosomatid and antimalarial therapeutics. Molecular and Biochemical Parasitology 2003, 126, 155-163.
(12) Chakrabarti, D.; Da Silva, T.; Barger, J.; Paquette, S.; Patel, H. et al. Protein Farnesyltransferase and Protein Prenylation in Plasmodium falciparum. Journal of Biological Chemistry 2002, 277, 42066-42073.
(13) Ohkanda, J.; Lockman, J. W.; Yokoyama, K.; Gelb, M. H.; Croft, S. L. et al. Peptidomimetic inhibitors of protein farnesyltransferase show potent antimalarial activity. Bioorganic & Medicinal Chemistry Letters 2001, 11, 761-764.
(14) Wiesner, J.; Kettler, K.; Sakowski, J.; Ortmann, R.; Katzin Alejandro, M. et al. Farnesyltransferase inhibitors inhibit the growth of malaria parasites in vitro and in vivo. Angewandte Chemie (International ed. in English) 2004, 43, 251-254.
(15) Sebti, S. M.; Hamilton, A. D. Farnesyltransferase and geranylgeranyltransferase I inhibitors and cancer therapy: lessons from mechanism and bench-to-bedside translational studies. Oncogene 2000, 19, 6584-6593.
(16) Strickland, C. L.; Windsor, W. T.; Syto, R.; Wang, L.; Bond, R. et al. Crystal Structure of Farnesyl Protein Transferase Complexed with a CaaX Peptide and Farnesyl Diphosphate Analog. Biochemistry 1998, 37, 16601-16611.
(17) Bahl, A.; Brunk, B.; Coppel, R. L.; Crabtree, J.; Diskin, S. J. et al. PlasmoDB: the Plasmodium genome resource. An integrated database providing tools for accessing, analyzing and mapping expression and sequence data (both finished and unfinished). Nucleic Acids Research 2002, 30, 87-90.
(18) Kissinger, J. C.; Brunk, B. P.; Crabtree, J.; Fraunholz, M. J.; Gajria, B. et al. The Plasmodium genome database. Nature (London, United Kingdom) 2002, 419, 490-492.
(19) Notredame, C.; Higgins, D. G.; Hering a, J. T-Coffee: A Novel Method for Fast and Accurate Multiple Sequence Alignment. Journal of Molecular Biology 2000, 302, 205-217.
(20) Sanchez, R.; SalI, A. Advances in comparative protein-structure modeling. Current Opinion in Structural Biology 1997, 7, 206-214.
(21) Feller, S. E.; Gawrisch, K.; MacKerell, A. D., Jr. Polyunsaturated Fatty Acids in Lipid Bilayers: Intrinsic and Environmental Contributions to Their Unique Physical Properties. Journal of the American Chemical Society 2002, 124, 318-326.
(22) Jones, G.; Willett, P.; Glen, R. C.; Leach, A. R.; Taylor, R. Development and validation of a genetic algorithm for flexible docking. Journal of Molecular Biology 1997, 267, 727-748.
(23) Eastman, R. T.; White, J.; Hucke, O.; Bauer, K.; Yokoyama, K. et al. Resistance to a Protein Farnesyltransferase Inhibitor in Plasmodium falciparum. Journal of Biological Chemistry 2005, 280, 13554-13559.
(24) Irvine, J. D.; Takahashi, L.; Lockhart, K.; Cheong, J.; Tolan, J. W. et al. MDCK (Madin-Darby canine kidney) cells: a tool for membrane permeability screening. Journal of Pharmaceutical Sciences 1999, 88, 28-33.
(25) Artursson, P.; Karlsson, J. Correlation between oral drug absorption in humans and apparent drug permeability coefficients in human intestinal epithelial (Caco-2) cells. Biochemical and Biophysical Research Communications 1991, 175, 880-885.
(26) Trager, W.; Jensen, J. B. Human malaria parasites in continuous culture. Science 1976, 193, 673-675.
(27) Van der Veken, P.; Senten, K.; Kertesz, I.; De Meester, I.; Lambeir, A.-M. et al. Fluoro-Olefins as Peptidomimetic Inhibitors of Dipeptidyl Peptidases. Journal of Medicinal Chemistry, ACS ASAP.

1. A compound according to the structure: 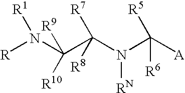
Where R¹ is an optionally substituted C₃-C₁₂ hydrocarbyl group (preferably a cyclic alkyl group), an optionally substituted heterocyclic group, an optionally substituted aromatic group or an optionally substituted heteroaromatic group;
R is a C(O)_{y}R′ group (preferably forming an optionally substituted C₂-C₅ acyl group), or a S(O)ₓR′ group, where y is 0 or 1 and x is 0, 1 or 2 and R′ is H or an optionally substituted C₁-C₁₂ alkyl group, or R′ is an optionally substituted C₅-C₁₂ cycloalkyl group, an optionally substituted heterocyclic group, an optionally substituted aromatic group or an optionally substituted heteroaromatic group;
R⁵, R⁶, R⁷, R⁸, R⁹ and R¹⁰ are each independently selected from H, an optionally substituted C₁-C₁₂ hydrocarbyl group, including a C₅-C₁₂ cycloalkyl group, an optionally substituted heterocyclic group, an optionally substituted aromatic group or an optionally substituted heteroaromatic group, or R⁵ and R⁶, R⁷ and R⁸ or R⁹ and R¹⁰ together form a keto (C═O) group;
R^{N} is H, an optionally substituted C₁-C₁₂ hydrocarbyl group, an optionally substituted heterocyclic group, an optionally substituted aromatic group, or an optionally substituted heteroaromatic group; 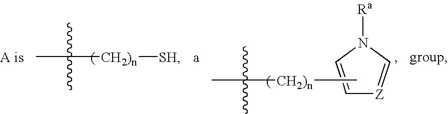
or a 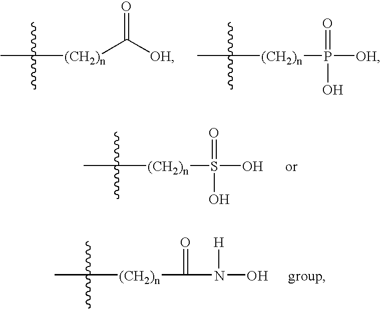
where Z is N, O or S;
R^{a} is H, a C₁-C₁₂ optionally substituted hydrocarbyl group or an optionally substituted aromatic group;
n is from 0 to 3; and pharmaceutically acceptable salts thereof.

2. The compound according to claim 1 wherein R¹ is an optionally substituted alkylene phenyl group or an optionally substituted heterocyclic or optionally substituted heteroaromatic group.

3. The compound according to claim 1 wherein R is a C₂-C₅ keto group or an SO₂R′ group.

4. The compound according to claim 3 wherein R′ is a substituted phenyl group, or an optionally substituted heteroaromatic group.

5. The compound according to claim 4 wherein R′ is an optionally substituted heteroaromatic group.

6. The compound according to claim 4 wherein R′ is a N-methylimidazole group.

7. The compound according to claim 1 wherein R^{a} is an alkyl group.

8. The compound according to claim 7 wherein R^{a} is a methyl group.

9. The compound according to claim 1 wherein R^{N} is an optionally substituted phenyl group.

10. The compound according to claim 1 wherein R^{N} is a phenyl group substituted with CN or at least one halogen.

11. The compound according to claim 1 wherein R⁵, R⁶, R⁷, R⁸, R⁹ and R¹⁰ are each H or up to three of R⁵, R⁶, R⁷, R⁸, R⁹ and R¹⁰ are CH₃.

12. The compound according to claim 11 wherein R⁵, R⁶, R⁷, R⁸, R⁹ and R¹⁰ are each H.

13. The compound according to claim 1 wherein R⁵ and R⁶, R⁷ and R⁸ or R⁸ and R⁹ together form a keto (C═O) group.

14. The compound according to claim 1 wherein R¹ is an optionally substituted alkylene phenyl group or an optionally substituted heterocyclic or optionally substituted heteroaromatic group; R is a C₂-C₅ keto group or an SO₂R′ group; R′ is a N-methylimidazole group; R^{a} is a methyl group; R^{N} is a phenyl group substituted with CN or at least one halogen; and R⁵, R⁶, R⁷, R⁸, R⁹ and R¹⁰ are each H, or up to three of R⁵, R⁶, R⁷, R⁸, R⁹ and R¹⁰ are CH₃.

15. A pharmaceutical composition comprising an effective amount of a compound according to claim 1, optionally in combination with a pharmaceutically acceptable carrier, additive or excipient.

16. A method of inhibiting the enzyme farnesyl transferase in a subject in need thereof comprising administering to said subject an amount of a compound according to claim 1, optionally in combination with a pharmaceutically acceptable carrier, additive or excipient effective to inhibit said enzyme in said subject.

17. A method of treating malaria in a patient comprising administering to said patient an effective amount of a compound according to claim 1 to said patient.

18. The method according to claim 17 wherein the causative agent of malaria in said patient is Plasmodium falciparum.

19. A method of treating neoplasia in a patient comprising administering to said patient an effective amount of a compound according to claim 1, optionally in combination with a pharmaceutically acceptable carrier, additive or excipient.

20. The method according to claim 19 wherein said neoplasm is a cancer.

21. The method according to claim 20 wherein said cancer is stomach, colon, rectal, liver, pancreatic, lung, breast, cervix uteri, corpus uteri, ovary, prostate, testis, bladder, renal, brain/cns, head and neck, throat, Hodgkins disease, non-Hodgkins leukemia, multiple myeloma leukemias, skin melanoma, acute lymphocytic leukemia, acute mylogenous leukemia, Ewings Sarcoma, small cell lung cancer, choriocarcinoma, rhabdomyosarcoma, Wilms Tumor, neuroblastoma, hairy cell leukemia, mouth/pharynx, oesophagus, larynx, melanoma, kidney and lymphoma.

22. A method of treating a hyperproliferative disease state in a patient in need of treatment comprising administering an effective amount of a compound according to claim 1 to said patient, optionally in combination with a pharmaceutically acceptable carrier, additive or excipient.

23. The method according to claim 22 wherein said hyperproliferative disease state is psoriasis, genital warts or a hyperproliferative cell growth disease.

24. The method according to claim 23 wherein said hyperproliferative cell growth disease is a hyperproliferative keratinocyte disease.

25. The method according to claim 24 wherein said hyperproliferative keratinocyte disease is hyperkeratosis, ichthyosis, keratoderma or lichen planus.

26. A method of treating arthritis in a patient comprising administering to said patient an effective amount of a compound according to claim 1, optionally in combination with a pharmaceutically acceptable carrier, additive or excipient.

27. The method according to claim 26 wherein said arthritis is rheumatoid arthritis or osteoarthritis.

28.-33. (canceled)

34. A compound according to claim 1 according to the chemical structure: 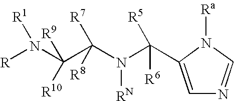
Where R^{a} is H or a C₁-C₆ optionally substituted hydrocarbyl group;
R¹ is an optionally substituted C₃-C₁₂ hydrocarbyl group, an optionally substituted heterocyclic group, an optionally substituted aromatic group or an optionally substituted heteroaromatic group;
R is a C(O)_{y}R′ group or a S(O)ₓR′ group, where y is 0 or 1 and x is 0, 1 or 2 and R′ is H or an optionally substituted C₁-C₁₂ alkyl group, or R′ is an optionally substituted C₅-C₁₂ cycloalkyl group, an optionally substituted heterocyclic group, an optionally substituted aromatic group or an optionally substituted heteroaromatic group;
R⁵, R⁶, R⁷, R⁸, R⁹ and R¹⁰ are each independently selected from H, an optionally substituted C₁-C₁₂ hydrocarbyl group, including a C₅-C₁₂ cycloalkyl group, an optionally substituted heterocyclic group, an optionally substituted aromatic group or an optionally substituted heteroaromatic group, or R⁵ and R⁶, R⁷ and R⁸ or R⁹ and R¹⁰ together form a keto (C═O) group; and
R^{N} is H, an optionally substituted C₁-C₁₂ hydrocarbyl group, an optionally substituted heterocyclic group, an optionally substituted aromatic group, or an optionally substituted heteroaromatic group, and pharmaceutically acceptable salts thereof.
